SEQUENCE LISTING (4 sequences)

SEQ ID NO: 1 - protein, 801 aa, Homo sapiens
MLQNVTPHNKLPGEGNAGLLGLGPEAAAPGKRIRKPSLLYEGFESPTMASVPALQLTPANPPPPEVSNPKKPGRVTNQLQYLHKVVMKALWKHQFAWPFRQPVDAVKLGLPDYHKIIKQPMDMGTIKRRLENNYYWAASECMQDFNTMFTNCYIYNKPTDDIVLMAQTLEKIFLQKVASMPQEEQELVVTIPKNSHKKGAKLAALQGSVTSAHQVPAVSSVSHTALYTPPPEIPTTVLNIPHPSVISSPLLKSLHSAGPPLLAVTAAPPAQPLAKKKGVKRKADTTTPTPTAILAPGSPASPPGSLEPKAARLPPMRRESGRPIKPPRKDLPDSQQQHQSSKKGKLSEQLKHCNGILKELLSKKHAAYAWPFYKPVDASALGLHDYHDIIKHPMDLSTVKRKMENRDYRDAQEFAADVRLMFSNCYKYNPPDHDVVAMARKLQDVFEFRYAKMPDEPLEPGPLPVSTAMPPGLAKSSSESSSEESSSESSSEEEEEEDEEDEEEEESESSDSEEERAHRLAELQEQLRAVHEQLAALSQGPISKPKRKREKKEKKKKRKAEKHRGRAGADEDDKGPRAPRPPQPKKSKKASGSGGGSAALGPSGFGPSGGSGTKLPKKATKTAPPALPTGYDSEEEEESRPMSYDEKRQLSLDINKLPGEKLGRVVHIIQAREPSLRDSNPEEIEIDFETLKPSTLRELERYVLSCLRKKPRKPYTIKKPVGKTKEELALEKKRELEKRLQDVSGQLNSTKKPPKKANEKTESSSAQQVAVSRLSASSSSSDSSSSSSSSSSSDTSDSDSG

SEQ ID NO: 2 - protein, 726 aa, Homo sapiens
MSTATTVAPAGIPATPGPVNPPPPEVSNPSKPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMPQEEVELLPPAPKGKGRKPAAGAQSAGTQQVAAVSSVSPATPFQSVPPTVSQTPVIAATPVPTITANVTSVPVPPAAAPPPPATPIVPVVPPTPPVVKKKGVKRKADTTTPTTSAITASRSESPPPLSDPKQAKVVARRESGGRPIKPPKKDLEDGEVPQHAGKKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMPDEPVEAPALPAPAAPMVSKGAESSRSSEESSSDSGSSDSEEERATRLAELQEQLKAVHEQLAALSQAPVNKPKKKKEKKEKEKKKKDKEKEKEKHKVKAEEEKKAKVAPPAKQAQQKKAPAKKANSTTTAGRQLKKGGKQASASYDSEEEEEGLPMSYDEKRQLSLDINRLPGEKLGRVVHIIQSREPSLRDSNPDEIEIDFETLKPTTLRELERYVKSCLQKKQRKPFSASGKKQAAKSKEELAQEKKKELEKRLQDVSGQLSSSKKPARKEKPGSAPSGGPSRLSSSSSSESGSSSSSGSSSDSSDSE

SEQ ID NO: 3 - protein, 722 aa, Homo sapiens
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETGPA

SEQ ID NO: 4 - protein, 947 aa, Homo sapiens
MSLPSRQTAIIVNPPPPEYINTKKNGRLTNQLQYLQKVVLKDLWKHSFSWPFQRPVDAVKLQLPDYYTIIKNPMDLNTIKKRLENKYYAKASECIEDFNTMFSNCYLYNKPGDDIVLMAQALEKLFMQKLSQMPQEEQVVGVKERIKKGTQQNIAVSSAKEKSSPSATEKVFKQQEIPSVFPKTSISPLNVVQGASVNSSSQTAAQVTKGVKRKADTTTPATSAVKASSEFSPTFTEKSVALPPIKENMPKNVLPDSQQQYNVVKTVKVTEQLRHCSEILKEMLAKKHFSYAWPFYNPVDVNALGLHNYYDVVKNPMDLGTIKEKMDNQEYKDAYKFAADVRLMFMNCYKYNPPDHEVVTMARMLQDVFETHFSKIPIEPVESMPLCYIKTDITETTGRENTNEASSEGNSSDDSEDERVKRLAKLQEQLKAVHQQLQVLSQVPFRKLNKKKEKSKKEKKKEKVNNSNENPRKMCEQMRLKEKSKRNQPKKRKQQFIGLKSEDEDNAKPMNYDEKRQLSLNINKLPGDKLGRVVHIIQSREPSLSNSNPDEIEIDFETLKASTLRELEKYVSACLRKRPLKPPAKKIMMSKEELHSQKKQELEKRLLDVNNQLNSRKRQTKSDKTQPSKAVENVSRLSESSSSSSSSSESESSSSDLSSSDSSDSESEMFPKFTEVKPNDSPSKENVKKMKNECILPEGRTGVTQIGYCVQDTTSANTTLVHQTTPSHVMPPNHHQLAFNYQELEHLQTVKNISPLQILPPSGDSEQLSNGITVMHPSGDSDTTMLESECQAPVQKDIKIKNADSWKSLGKPVKPSGVMKSSDELFNQFRKAAIEKEVKARTQELIRKHLEQNTKELKASQENQRDLGNGLTVESFSNKIQNKCSGEEQKEHQQSSEAQDKSKLWLLKDRDLARQKEQERRRREAMVGTIDMTLQSDIMTMFENNFD

(19) United States
(12) Patent Application Publication
(10) Pub. No.: US 2013/0252331 A1
(43) Pub. Date: Sep. 26, 2013

(54) COMPOSITIONS AND METHODS FOR MODULATING METABOLISM

(71) Applicants: James Elliott Bradner, Boston, MA (US); Jonathan Brown, Brookline, MA (US); Jorge Plutzky, Chestnut Hill, MA (US)

(72) Inventors: James Elliott Bradner, Boston, MA (US); Jonathan Brown, Brookline, MA (US); Jorge Plutzky, Chestnut Hill, MA (US)

(21) Appl. No.: 13/697,963

(22) PCT Filed: May 16, 2011
(86) PCT No.: PCT/US11/36647

§ 371 Date: Jun. 3, 2013

Related U.S. Application Data
(60) Provisional application No. 61/334,991, filed on May 14, 2010.
(60) Provisional application No. 61/370,745, filed on Aug. 4, 2010.
(60) Provisional application No. 61/375,863, filed on Aug. 22, 2010.
(60) Provisional application No. 61/467,321, filed on Mar. 24, 2011.
(60) Provisional application No. 61/467,376, filed on Mar. 24, 2011.

(51) Int. Cl. C07D 495/14; A61K 31/7088

(52) U.S. Cl. CPC C07D 495/14; A61K 31/7088
USPC 435/375; 514/220; 540/560

(57) ABSTRACT

The invention provides compositions comprising an effective amount of an agent that inhibits a BET protein (e.g., Brd2, Brd3, Brd4), and methods of using such compositions for treating or preventing metabolic syndrome, obesity, type II diabetes, insulin resistance, and related disorders characterized by undesirable alterations in metabolism or fat accumulation.

### RELATED APPLICATIONS

This application claims priority to U.S. Provisional Patent Application Nos. 61/334,991, filed May 14, 2010; 61/370,745, filed Aug. 4, 2010; 61/375,863, filed Aug. 22, 2010; 61/467,376, filed Mar. 24, 2011; and 61/467,321, filed Mar. 24, 2011. The contents of each of these applications are incorporated herein by this reference in their entirety.

### STATEMENT OF RIGHTS TO INVENTIONS MADE UNDER FEDERALLY SPONSORED RESEARCH

This work was supported by the following grants from the National Institutes of Health, Grant Nos: K08CA128972 (Bradner); K08HL105678-01 (Brown). The government has certain rights in the invention.

### BACKGROUND OF THE INVENTION

Metabolic syndrome and obesity represent major health problems in all industrialized countries. Metabolic syndrome is a cluster of heart disease and diabetes risk factors that occur together and increase a patient's risk for serious disease, including heart disease, stroke and diabetes. The underlying risk factors for metabolic syndrome include insulin resistance and abdominal obesity. Obesity is the most significant nutritional disorder in the western world with estimates of its prevalence ranging from 30% to 50%. Obesity correlates with increased incidences of coronary artery disease, stroke, and type II diabetes. Obesity is not primarily merely a behavioral problem. Rather, the differential body composition observed between obese and normal subjects results from differences in both metabolism and neurologic/metabolic interactions. These differences seem to be, to some extent, due to differences in gene expression, and/or level of gene products or activity. The nature of the genetic factors that control body composition are unknown. Given the severity and prevalence of metabolism syndrome and obesity there exists a great need for compositions and methods for treating and preventing metabolic syndrome, obesity, type II diabetes, insulin resistance, and related disorders characterized by undesirable alterations in metabolism or fat accumulation.

### SUMMARY OF THE INVENTION

As described below, the present invention features compositions and methods for treating and/or preventing a metabolic syndrome, obesity, type II diabetes, insulin resistance, and related disorders characterized by undesirable alterations in metabolism or fat accumulation.

In one aspect, the invention provides a method of inhibiting adipogenesis, the method involving contacting an adipocyte or pre-adipocyte with an effective amount of an agent that inhibits a bromodomain and extra-terminal (BET) protein.

In another aspect, the invention provides a method of inhibiting adipocyte biological function, the method involving contacting an adipocyte with an effective amount of an agent that inhibits a bromodomain and extra-terminal (BET) protein.

In yet another aspect, the invention provides a method for treating or preventing metabolic syndrome in a human, the method involving administering to the human an effective amount of an agent that inhibits a bromodomain and extra-terminal (BET) protein, thereby treating or preventing metabolic syndrome in the human.

In further aspects, the invention provides a method for treating or preventing obesity or weight gain in a human, the method involving administering to the human an effective amount of an agent that inhibits a bromodomain and extra-terminal (BET) protein, thereby treating or preventing obesity or weight gain in the human.

In another aspect, the invention provides a method of inhibiting hepatic steatosis in a human, the method involving administering to the human an effective amount of an agent that inhibits a bromodomain and extra-terminal (BET) protein, thereby inhibiting hepatic steatosis.

In a further aspect, the invention provides a method of reducing subcutaneous fat or visceral fat in a human, the method involving administering to the human an effective amount of an agent that inhibits a bromodomain and extra-terminal (BET) protein, thereby reducing subcutaneous fat or visceral fat in the human.

In yet another aspect, the invention provides a method of inhibiting food intake or increasing metabolism in a human, the method involving administering to the human an effective amount of an agent that inhibits a bromodomain and extra-terminal (BET) protein, thereby inhibiting food intake or increasing metabolism in the human.

In an additional aspect, the invention provides a kit for the treatment of a body weight disorder, the kit comprising an effective amount of an inhibitor of bromodomain and extra-terminal (BET) protein and direction for use of the kit to practice any of the methods disclosed herein.

In various embodiments of the above aspects or any other aspect of the invention delineated herein, the method inhibits adipocyte differentiation, proliferation, or hypertrophy. In another embodiment the method reduces fatty acid synthesis, lipogenesis, lipid droplet accumulation. In further embodiments the method reduces abdominal obesity, atherogenic dyslipidemia, elevated blood pressure, insulin resistance, or type II diabetes. In other embodiments the agent is a compound of any of Formulas I-XXII, or any other compound herein, or a derivative thereof. In yet another embodiment the compound is JQ1. In additional embodiments the agent is an inhibitory nucleic acid molecule. In yet another embodiment the inhibitory nucleic acid molecule is an siRNA, shRNA or antisense nucleic acid molecule that reduces the expression of Brd2, Brd3, or Brd4. In other embodiments the bromodomain and extra-terminal (BET) protein is Brd2, Brd3, or Brd4. In a further embodiment the method reduces the level of a C/EBPα and/or PPARγ polypeptide or polynucleotide. In further embodiments the method reduces the level of a sterol regulatory binding protein (SREBP), peroxisome proliferator activated receptor 2 (PPARg2), fatty acid synthase (FAS), acetyl CoA carboxylase beta, stearoyl CoA desaturase 1 (SCD1), and diacyglycerol acyl transferase 1 (DGAT). In yet additional embodiments the agent is administered locally or systemically. In another embodiment the inhibitor of bromodomain and extra-terminal (BET) protein is JQ1.

The invention provides compositions comprising an effective amount of a BET family inhibitor, and methods of using such compositions for treating or preventing metabolic syndrome, obesity, type II diabetes, insulin resistance, and related disorders characterized by undesirable alterations in metabolism or fat accumulation. Other features and advantages of the invention will be apparent from the detailed description, and from the claims.

### DEFINITIONS

By “adipogenesis” is meant an increase in the number of adipocytes. Adipogenesis typically involves hyperplasia (increase in number) of adipocytes. Adipocyte hypertrophy is the increase in size of a pre-existing adipocyte as a result of excess triglyceride accumulation. Hypertrophy occurs when energy intake exceeds energy expenditure. Hyperplasia results from the formation of new adipocytes from precursor cells in adipose tissue. Typically hyperplasia involves the proliferation of preadipocytes and their differentiation into adipocytes.

By “body weight disorder” is meant any disorder or disease that results in an abnormal body weight.

By “inhibitor of bromodomain and extra-terminal (BET) protein” is meant any agent that inhibits or decreases the activity of a BET protein family member.

By “JQ1” is meant (+)-JQ1 ((S)-tert-Butyl 2-(4-(4-chlorophenyl)-2,3,9-trimethyl-6H-thieno[3,2-f][1,2,4]triazolo[4,3-a][1,4]diazepin-6-yl)acetate) as described herein

By “metabolic syndrome” is meant one or more risk factors that increase a subject's propensity to develop coronary heart disease, stroke, peripheral vascular disease and/or type II diabetes. Risk factors associated with metabolic syndrome include abdominal obesity (i.e, excessive fat tissue in and around the abdomen, atherogenic dyslipidemia including but not limited to high triglycerides, low HDL cholesterol and high LDL cholesterol, elevated blood pressure, insulin resistance or glucose intolerance, Prothrombotic state (e.g., high fibrinogen or plasminogen activator inhibitor-1 in the blood), proinflammatory state (e.g., elevated C-reactive protein in the blood). Agents of the invention are useful for the treatment or prevention of metabolic syndrome in a subject having one or more of the aforementioned risk factors.

By “obesity” is meant an excess of body fat relative to lean body mass. A subject is considered obese if they have a body mass index (BMI) of 30 and above.

By “body mass index (BMI)” is a subject's weight in kilograms divided by their height in meters squared.

By “weight gain” is meant an increase in body weight relative to the body weight of the individual at an earlier point in time or relative to a reference body weight. In one embodiment, a reference body weight corresponds to a BMI of about 25.

By “bromodomain” is meant a portion of a polypeptide that recognizes acetylated lysine residues. In one embodiment, a bromodomain of a BET family member polypeptide comprises approximately 110 amino acids and shares a conserved fold comprising a left-handed bundle of four alpha helices linked by diverse loop regions that interact with chromatin.

By “BET family polypeptide” is meant a polypeptide comprising two bromodomains and an extraterminal (ET) domain or a fragment thereof having transcriptional regulatory activity or acetylated lysine binding activity. Exemplary BET family members include BRD2, BRD3, BRD4 and BRDT.

By “BRD2 polypeptide” is meant a protein or fragment thereof having at least 85% identity to NP_{—}005095 that is capable of binding chromatin or regulating transcription.

The sequence of an exemplary BRD2 polypeptide follows:

MLQNVTPHNKLPGEGNAGLLGLGPEAAAPGKRIRKPSLLYEGFESPTMASVPALQLTPANPPPPEVSNPK
KPGRVTNQLQYLHKVVMKALWKHQFAWPFRQPVDAVKLGLPDYHKIIKQPMDMGTIKRRLENNYYWAASE
CMQDFNTMFTNCYIYNKPTDDIVLMAQTLEKIFLQKVASMPQEEQELVVTIPKNSHKKGAKLAALQGSVT
SAHQVPAVSSVSHTALYTPPPEIPTTVLNIPHPSVISSPLLKSLHSAGPPLLAVTAAPPAQPLAKKKGVK
RKADTTTPTPTAILAPGSPASPPGSLEPKAARLPPMRRESGRPIKPPRKDLPDSQQQHQSSKKGKLSEQL
KHCNGILKELLSKKHAAYAWPFYKPVDASALGLHDYHDIIKHPMDLSTVKRKMENRDYRDAQEFAADVRL
MFSNCYKYNPPDHDVVAMARKLQDVFEFRYAKMPDEPLEPGPLPVSTAMPPGLAKSSSESSSEESSSESS
SEEEEEEDEEDEEEEESESSDSEEERAHRLAELQEQLRAVHEQLAALSQGPISKPKRKREKKEKKKKRKA
EKHRGRAGADEDDKGPRAPRPPQPKKSKKASGSGGGSAALGPSGFGPSGGSGTKLPKKATKTAPPALPTG
YDSEEEEESRPMSYDEKRQLSLDINKLPGEKLGRVVHIIQAREPSLRDSNPEEIEIDFETLKPSTLRELE
RYVLSCLRKKPRKPYTIKKPVGKTKEELALEKKRELEKRLQDVSGQLNSTKKPPKKANEKTESSSAQQVA
VSRLSASSSSSDSSSSSSSSSSSDTSDSDSG

By “BRD2 nucleic acid molecule” is meant a polynucleotide encoding a BRD2 polypeptide or fragment thereof.

By “BRD3 polypeptide” is meant a protein or fragment thereof having at least 85% identity to NP_{—}031397.1 that is capable of binding chromatin or regulating transcription.

The sequence of an exemplary BRD3 polypeptide follows:

| | |
|---|---|
| 1 | mstattvapa gipatpgpvn ppppevsnps kpgrktnqlq ymqnvvvktl wkhqfawpfy |
| 61 | qpvdaiklnl pdyhkiiknp mdmgtikkrl ennyywsase cmqdfntmft ncyiynkptd |
| 121 | divlmaqale kiflqkvaqm pqeevellpp apkgkgrkpa agaqsagtqq vaavssvspa |
| 181 | tpfqsvpptv sqtpviaatp vptitanvts vpvppaaapp ppatpivpvv pptppvvkkk |
| 241 | gvkrkadttt pttsaitasr sesppplsdp kqakvvarre sggrpikppk kdledgevpq |
| 301 | hagkkgklse hlrycdsilr emlskkhaay awpfykpvda ealelhdyhd iikhpmdlst |
| 361 | vkrkmdgrey pdaqgfaadv rlmfsncyky nppdhevvam arklqdvfem rfakmpdepv |
| 421 | eapalpapaa pmvskgaess rsseesssds gssdseeera trlaelqeql kavheqlaal |
| 481 | sqapvnkpkk kkekkekekk kkdkekekek hkvkaeeekk akvappakqa qqkkapakka |
| 541 | nstttagrql kkggkqasas ydseeeeegl pmsydekrql sldinrlpge klgrvvhiiq |
| 601 | srepslrdsn pdeieidfet lkpttlrele ryvksclqkk qrkpfsasgk kqaakskeel |
| 661 | aqekkkelek rlqdvsgqls sskkparkek pgsapsggps rlsssssses gsssssgsss |
| 721 | dssdse |

By “Brd3 nucleic acid molecule” is meant a polynucleotide encoding a BRD3 polypeptide.

By “BRD4 polypeptide” is meant a protein or fragment thereof having at least 85% identity to NP_{—}055114 that is capable of binding chromatin or regulating transcription.

| | |
|---|---|
| 1 | msaesgpgtr lrnlpvmgdg letsqmsttq aqaqpqpana astnppppet snpnkpkrqt |
| 61 | nqlqyllrvv lktlwkhqfa wpfqqpvdav klnlpdyyki iktpmdmgti kkrlennyyw |
| 121 | naqeciqdfn tmftncyiyn kpgddivlma ealeklflqk inelpteete imivqakgrg |
| 181 | rgrketgtak pgvstvpntt qastppqtqt pqpnpppvqa tphpfpavtp dlivqtpvmt |
| 241 | vvppqplqtp ppvppqpqpp papapqpvqs hppiiaatpq pvktkkgvkr kadtttptti |
| 301 | dpiheppslp pepkttklgq rressrpvkp pkkdvpdsqq hpapeksskv seqlkccsgi |
| 361 | lkemfakkha ayawpfykpv dvealglhdy cdiikhpmdm stiksklear eyrdaqefga |
| 421 | dvrlmfsncy kynppdhevv amarklqdvf emrfakmpde peepvvayss pavppptkvv |
| 481 | appsssdsss dsssdsdsst ddseeeraqr laelqeqlka vheqlaalsq pqqnkpkkke |
| 541 | kdkkekkkek hkrkeeveen kkskakeppp kktkknnssn snvskkepap mkskppptye |
| 601 | seeedkckpm syeekrqlsl dinklpgekl grvvhiiqsr epslknsnpd eieidfetlk |
| 661 | pstlrelery vtsclrkkrk pqaekvdvia gsskmkgfss sesesssess ssdsedsetg |
| 721 | pa |

By “Brd4 nucleic acid molecule” is meant a polynucleotide that encodes a BRD4 polypeptide.

By “BRDT polypeptide” is meant a protein or fragment thereof having at least 85% identity to NP_{—}001717 that is capable of binding chromatin or regulating transcription.

| | |
|---|---|
| 1 | mslpsrqtai ivnppppeyi ntkkngrltn qlqylqkvvl kdlwkhsfsw pfqrpvdavk |
| 61 | lqlpdyytii knpmdlntik krlenkyyak aseciedfnt mfsncylynk pgddivlmaq |
| 121 | aleklfmqkl sqmpqeeqvv gvkerikkgt qqniavssak eksspsatek vfkqqeipsv |
| 181 | fpktsispln vvqgasvnss sqtaaqvtkg vkrkadtttp atsavkasse fsptfteksv |
| 241 | alppikenmp knvlpdsqqq ynvvktvkvt eqlrhcseil kemlakkhfs yawpfynpvd |
| 301 | vnalglhnyy dvvknpmdlg tikekmdnqe ykdaykfaad vrlmfmncyk ynppdhevvt |
| 361 | marmlqdvfe thfskipiep vesmplcyik tditettgre ntneassegn ssddsederv |
| 421 | krlaklqeql kavhqqlqvl sqvpfrklnk kkekskkekk kekvnnsnen prkmceqmrl |
| 481 | kekskrnqpk krkqqfiglk sedednakpm nydekrqlsl ninklpgdkl grvvhiiqsr |
| 541 | epslsnsnpd eieidfetlk astlreleky vsaclrkrpl kppakkimms keelhsqkkq |
| 601 | elekrlldvn nqlnsrkrqt ksdktqpska venvsrlses sssssssses essssdlsss |
| 661 | dssdsesemf pkftevkpnd spskenvkkm knecilpegr tgvtqigycv qdttsanttl |
| 721 | vhqttpshvm ppnhhqlafn yqelehlqtv knisplqilp psgdseqlsn gitvmhpsgd |
| 781 | sdttmlesec qapvqkdiki knadswkslg kpvkpsgvmk ssdelfnqfr kaaiekevka |
| 841 | rtqelirkhl eqntkelkas qenqrdlgng ltvesfsnki qnkcsgeeqk ehqqsseaqd |
| 901 | ksklwllkdr dlarqkeqer rrreamvgti dmtlqsdimt mfennfd |

By “BRDT nucleic acid molecule” is meant a polynucleotide encoding a BRDT polypeptide.

By “compound” is meant any small molecule chemical compound, antibody, nucleic acid molecule, or polypeptide, or fragments thereof.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “halogen” designates —F, —Cl, —Br or —I.

The term “haloalkyl” is intended to include alkyl groups as defined herein that are mono-, di- or polysubstituted by halogen, e.g., fluoromethyl and trifluoromethyl.

The term “hydroxyl” means —OH.

The term “heteroatom” as used herein means an atom of any element other than carbon or hydrogen. Preferred heteroatoms are nitrogen, oxygen, sulfur and phosphorus.

As used herein, the term “alkyl” means a saturated straight chain or branched non-cyclic hydrocarbon typically having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be unsubstituted, or optionally substituted with one or more substituents, such as amino, alkylamino, arylamino, heteroarylamino, alkoxy, alkylthio, oxo, halo, acyl, nitro, hydroxyl, cyano, aryl, heteroaryl, alkylaryl, alkylheteroaryl, aryloxy, heteroaryloxy, arylthio, heteroarylthio, arylamino, heteroarylamino, carbocyclyl, carbocyclyloxy, carbocyclylthio, carbocyclylamino, heterocyclyl, heterocyclyloxy, heterocyclylamino, heterocyclylthio, and the like. Lower alkyls are typically preferred for the compounds of this invention.

As used herein, the term an “aromatic ring” or “aryl” means a monocyclic or polycyclic-aromatic ring or ring radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or optionally is substituted with one or more substituents, e.g., substituents as described herein for alkyl groups (including without limitation alkyl (preferably, lower alkyl or alkyl substituted with one or more halo), hydroxy, alkoxy (preferably, lower alkoxy), alkylthio, cyano, halo, amino, boronic acid (—B(OH)₂, and nitro). In certain embodiments, the aryl group is a monocyclic ring, wherein the ring comprises 6 carbon atoms.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon. Heteroaryl groups may be optionally substituted with one or more substituents, e.g. as for aryl groups as described herein. Examples of heteroaryl groups include, but are not limited to, pyridyl, furanyl, benzodioxolyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, and indolyl.

The term “heterocyclic” as used herein, refers to organic compounds that contain at least at least one atom other than carbon (e.g., S, O, N) within a ring structure. The ring structure in these organic compounds can be either aromatic or, in certain embodiments, non-aromatic. Some examples of heterocyclic moeities include, are not limited to, pyridine, pyrimidine, pyrrolidine, furan, tetrahydrofuran, tetrahydrothiophene, and dioxane.

The term “isomers” or “stereoisomers” refers to compounds which have identical chemical constitution, but differ with regard to the arrangement of the atoms or groups in space.

The term “isotopic derivatives” includes derivatives of compounds in which one or more atoms in the compounds are replaced with corresponding isotopes of the atoms. For example, an isotopic derivative of a compound containg a carbon atom (C¹²) would be one in which the carbon atom of the compound is replaced with the C¹³ isotope.

By “computer modeling” is meant the application of a computational program to determine one or more of the following: the location and binding proximity of a ligand to a binding moiety, the occupied space of a bound ligand, the amount of complementary contact surface between a binding moiety and a ligand, the deformation energy of binding of a given ligand to a binding moiety, and some estimate of hydrogen bonding strength, van der Waals interaction, hydrophobic interaction, and/or electrostatic interaction energies between ligand and binding moiety. Computer modeling can also provide comparisons between the features of a model system and a candidate compound. For example, a computer modeling experiment can compare a pharmacophore model of the invention with a candidate compound to assess the fit of the candidate compound with the model.

By a “computer system” is meant the hardware means, software means and data storage means used to analyse atomic coordinate data. The minimum hardware means of the computer-based systems of the present invention comprises a central processing unit (CPU), input means, output means and data storage means. Desirably a monitor is provided to visualise structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

By “computer readable media” is meant any media which can be read and accessed directly by a computer e.g. so that the media is suitable for use in the above-mentioned computer system. The media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases susceptible to treatment with compounds delineated herein include metabolic syndrome, obesity, type II diabetes, insulin resistance, and related disorders characterized by undesirable alterations in metabolism or fat accumulation.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “enantiomers” refers to two stereoisomers of a compound which are non-superimposable mirror images of one another. An equimolar mixture of two enantiomers is called a “racemic mixture” or a “racemate.”

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl.

The term “hydroxyl” means —OH.

The term “heteroatom” as used herein means an atom of any element other than carbon or hydrogen. Preferred heteroatoms are nitrogen, oxygen, sulfur and phosphorus.

The term “heterocyclic” as used herein, refers to organic compounds that contain at least at least one atom other than carbon (e.g., S, O, N) within a ring structure. The ring structure in these organic compounds can be either aromatic or non-aromatic. Some examples of heterocyclic moeities include, are not limited to, pyridine, pyrimidine, pyrrolidine, furan, tetrahydrofuran, tetrahydrothiophene, and dioxane.

The term “isomers” or “stereoisomers” refers to compounds which have identical chemical constitution, but differ with regard to the arrangement of the atoms or groups in space.

The term “isotopic derivatives” includes derivatives of compounds in which one or more atoms in the compounds are replaced with corresponding isotopes of the atoms. For example, an isotopic derivative of a compound containg a carbon atom (C¹²) would be one in which the carbon atom of the compound is replaced with the C¹³ isotope.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fitting” is meant determining by automatic, or semi-automatic means, interactions between one or more atoms of an agent molecule and one or more atoms or binding sites of a BET family member (e.g., a bromodomain of BRD2, BRD3, BRD4 and BRDT), and determining the extent to which such interactions are stable. Various computer-based methods for fitting are described further herein.

The term “optical isomers” as used herein includes molecules, also known as chiral molecules, that are exact non-superimposable mirror images of one another.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “polymorph” as used herein, refers to solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included. Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. Both configurations, cis/trans and/or Z/E are encompassed by the compounds of the present invention.

With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. By “substantially identical” is meant a polypeptide or nucleic acid molecule exhibiting at least 85% identity to a reference amino acid sequence (for example, any one of the amino acid sequences described herein) or nucleic acid sequence (for example, any one of the nucleic acid sequences described herein). Preferably, such a sequence is at least 85%, 90%, 95%, 99% or even 100% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e.sup.-3 and e.sup.-100 indicating a closely related sequence.

By “increases” is meant a positive alteration of at least about 10%, 25%, 50%, 75%, or 100% relative to a reference.

By “root mean square deviation” is meant the square root of the arithmetic mean of the squares of the deviations from the mean.

By “subject” is meant a mammal, including, but not limited to, a human or non-human mammal, such as a bovine, equine, canine, ovine, or feline.

By “specifically binds” is meant a compound or antibody that recognizes and binds a polypeptide of the invention, but which does not substantially recognize and bind other molecules in a sample, for example, a biological sample, which naturally includes a polypeptide of the invention.

The term “sulfhydryl” or “thiol” means —SH.

As used herein, the term “tautomers” refers to isomers of organic molecules that readily interconvert by tautomerization, in which a hydrogen atom or proton migrates in the reaction, accompanied in some occasions by a switch of a single bond and an adjacent double bond.

As used herein, the terms “treat,” treating,” “treatment,” and the like refer to reducing or ameliorating a disorder and/or symptoms associated therewith. By “ameliorate” is meant decrease, suppress, attenuate, diminish, arrest, or stabilize the development or progression of a disease. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

“An effective amount” refers to an amount of a compound, which confers a therapeutic effect on the treated subject. The therapeutic effect may be objective (i.e., measurable by some test or marker) or subjective (i.e., subject gives an indication of or feels an effect). An effective amount of a compound described herein may range from about 1 mg/Kg to about 5000 mg/Kg body weight. Effective doses will also vary depending on route of administration, as well as the possibility of co-usage with other agents.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 includes eight micrographs showing that the inhibition of BET protein family members blocks adipogenesis in a dose dependent manner in 3T3L1 cells, which is a cell line that is widely used as a model for adipogenesis. Cells were treated with various doses of the active JQ1 (S) enantiomer or the inactive control JQ1 (R) enantiomer. Following drug treatment cells were stained with Oil Red O as a measure of lipid accumulation that indicates the degree of adipocyte differentiation. As shown, the JQ1 (S) enantiomer inhibited lipid accumulation.

FIGS. 2A and 2B are graphs showing that inhibition of BET protein family members blocks the expression of C/EBPα and PPARγ in 3T3L1 cells during adipocyte differentiation. C/EBPα and PPARγ are essential, positive regulators of adipogenesis. FIG. 2A is a graph of C/EBPα expression levels over time in control and JQ1 treated 3T3L1 cells. FIG. 2B is a graph of PPARγ expression levels over time in control and JQ1 treated 3T3L1 cells. Results were obtained using RT-PCR.

FIGS. 3A-3E are graphs showing that inhibition of BET family members blocks weight gain in ob/ob mice, a murine obesity model that lacks leptin. FIG. 3A is a graph of body weight of ob/ob mice before and after 14 days of treatment with JQ1. FIG. 3B is a graph of body weight over time in control and JQ1 treated ob/ob mice. FIG. 3C is a graph showing total weight gain during the 14 day treatment period in control treated and JQ1 treated ob/ob mice. FIG. 3D is a plot of total food intake during the 14 day treatment period in control treated and JQ1 treated ob/ob mice. FIG. 3E is a plot of feed efficiency during the 14 day treatment period in control treated and JQ1 treated ob/ob mice. Importantly, JQ1 blocked weight gain in the ob/ob mice relative to control mice.

FIGS. 4A and 4B are graphs showing that inhibition of BET protein family members reduces liver and adipose tissue weight in ob/ob mice. FIG. 4A quantitates liver weight in ob/ob mice treated with vehicle or JQ1. FIG. 4B quantitates subcutaneous fat weight in ob/ob mice treated with vehicle or JQ1.

FIG. 5 includes two micrographs showing that inhibition of BET protein family members completely blocks the formation of fatty liver in a mouse obesity model. The sections were stained with hematoxylin and eosin. Large lipid droplets are prevalent in the section obtained from an ob/ob mouse that received vehicle alone. Significantly, liver morphology is normal in the mouse that treated with JQ1.

FIGS. 6A-6F show that inhibition of BET protein family members reduces the expression of genes that control fat accumulation in liver. FIGS. 6A-6F are a panel of graphs that show gene expression in vehicle treated and JQ1 treated ob/ob mice. Interestingly, JQ11 reduced the expression of SREBP (FIG. 6A), PPARγ2 (FIG. 6B), FAS (this was not statistically significant) (FIG. 6C), ACC beta (FIG. 6D), SCD1 (FIG. 6E), and DGAT (FIG. 6F).

FIGS. 7A-7C show that bromodomain inhibition reduced visceral fat mass in mice fed a normal chow diet. FIG. 7A is a graph of body weight in mice fed normal chow over time in vehicle treated and JQ1 treated mice (50 mg/kg administered daily). FIG. 7B is a graph comparing visceral fat in mice after 8 weeks of either vehicle control or JQ1 treatment. FIG. 7C is a graph comparing subcutaneous fat in mice after 8 weeks of either vehicle control or JQ1 treatment.

FIG. 8 shows that bromodomain inhibition blocked weight gain in response to high fat diet. FIG. 8 is a graph of body weight of mice fed a high fat diet over time in vehicle treated and JQ1 treated mice (50 mg/kg administered daily).

FIGS. 9A & 9B show that bromodomain inhibition protects against insulin resistance after 8 weeks exposure to a high fat diet. FIG. 9A is a graph of blood glucose following insulin injection in mice that had been on a high fat diet for 7 weeks and treated daily with vehicle control or JQ1. FIG. 9B is a graph of the area under the curve (AUC) of the data in FIG. 9A.

### DETAILED DESCRIPTION OF THE INVENTION

The invention features compositions and methods that are useful for the treatment or prevention of metabolic syndrome, obesity, type II diabetes, insulin resistance, hepatic steatosis and related disorders characterized by undesirable alterations in metabolism or fat accumulation.

The invention is based, at least in part, on the discovery that agents that inhibit one or more members of the BET protein family block weight gain and negatively regulate a host of transcription factors that function in adipogenesis and also control lipid partitioning and ectopic accumulation of fat in other tissues such as liver and muscle. The BET family of proteins, which includes BRD1, BRD2, BRD3, BRD4, and BRDT, are important regulators of chromatin remodelling, and likely control adipocyte differentiation by reducing the expression of transcription factors, including SREBP and PPARγ2 as well as the target genes regulated by these transcription factors including fatty acid synthase (FAS), ACC beta, SCD1, and DGAT (Note: Technically the FAS data did not meet statistical significance). The results reported herein were obtained using a cell-permeable, potent small-molecule inhibitor (JQ1) with biochemical selectivity for the BET-family of bromodomains. The invention further provides for the use of related compounds capable of regulating the bromodomain family, which are a family of polypeptides that contain a bromodomain that recognizes acetyl-lysine residues on nuclear chromatin. Lysine acetylation has emerged as a signaling modification of broad relevance to cellular and disease biology. Targeting the enzymes which reversibly mediate side-chain acetylation has been an active area of drug discovery research for many years. To date, successful efforts have been limited to the “writers” (acetyltransferases) and “erasers” (histone deacetylases) of covalent modifications arising in the context of nuclear chromatin.

The recent characterization of a high-resolution co-crystal structures with BRD4 revealed excellent shape complementarity with the acetyl-lysine binding cavity. Binding of JQ1 to the tandem bromodomains of BRD4 is acetyl-lysine competitive and displaces BRD4 from chromatin in human cells. These data establish the feasibility of targeting protein-protein interactions of epigenetic “readers” to block adipocyte differentiation. Moreover, extended in vivo use of such compounds in a well established murine obesity model, the ob/ob mouse, showed that inhibition of BET proteins blocked weight gain, reduced adipose tissue weight, and inhibited fat accumulation in liver. In the liver, the decrease in fat accumulation was accompanied by a significant decrease in the expression of genes that control fat synthesis. The data reported herein establish that agents that inhibit BET proteins are potent inhibitors of obesity and related metabolic disorders, including fatty liver. Treatment of obesity and fatty liver is beneficial for metabolic syndrome, type II diabetes, insulin resistance, and related disorders characterized by undesirable alterations in metabolism or fat accumulation, and symptoms thereof.

### Metabolic Syndrome

Metabolic syndrome is a cluster of heart disease and diabetes risk factors that occur together and increase a patient's risk for serious disease, including heart disease, stroke and diabetes. In one embodiment, the criteria for metabolic syndrome include an increased waist circumference (abdominal obesity), elevated triglycerides, reduced high-density lipoprotein cholesterol (HDL-C), elevated blood pressure, and/or an elevated fasting glucose. In particular, levels of triglycerides of 150 mg/dL or higher; a high density lipoproteins (HDL) cholesterol lower than 40 mg/dL for men and lower than 50 mg/dL for women; a blood pressure level of 130/85 mm Hg or higher; or a fasting glucose of 100 mg/dL or higher. For most Americans, a waist circumference of 35 inches or more for women and 40 inches or more for men is considered abnormally increased. An individual who has abnormal levels of at least three of the listed criteria is considered to have metabolic syndrome. Many physicians believe that metabolic syndrome is likely associated with resistance to insulin. Metabolic syndrome increases the risk for atherosclerotic cardiovascular disease by 1.5-3 fold, and raises the risk for type 2 diabetes by 3-5 fold. It affects over 26 percent of adults, or over 50 million Americans.

Clinical management of metabolic syndrome is focused on reducing the risk for atherosclerotic cardiovascular disease, and the risk of type 2 diabetes in patients who have not yet developed clinical diabetes. Recently published results indicate that one in five adults in the U.S. has metabolic syndrome. Current methods of treating metabolic syndrome are inadequate. Compositions of the invention comprising agents that inhibit the biological activity of one or more BET proteins (e.g., Brd2, Brd3, Brd4) are useful for the prevention or treatment of a metabolic syndrome, or for the prevention or treatment of any one or more of the risk factors associated with a metabolic syndrome.

### Bromodomain-Containing Proteins

Gene regulation is fundamentally governed by reversible, non-covalent assembly of macromolecules. Signal transduction to RNA polymerase requires higher-ordered protein complexes, spatially regulated by assembly factors capable of interpreting the post-translational modification states of chromatin. Epigenetic readers are structurally diverse proteins each possessing one or more evolutionarily conserved effector modules, which recognize covalent modifications of histone proteins or DNA. The ε-N-acetylation of lysine residues (Kac) on histone tails is associated with an open chromatin architecture and transcriptional activation³. Context-specific molecular recognition of acetyl-lysine is principally mediated by bromodomains.

Bromodomain-containing proteins are of substantial biological interest, as components of transcription factor complexes (TAF1, PCAF, Gcn5 and CBP) and determinants of epigenetic memory⁴. There are 41 human proteins containing a total of 57 diverse bromodomains. Despite large sequence variations, all bromodomains share a conserved fold comprising a left-handed bundle of four alpha helices (α_{Z}, α_{A}, α_{B}, α_{C}), linked by diverse loop regions (ZA and BC loops) that determine substrate specificity. Co-crystal structures with peptidic substrates showed that the acetyl-lysine is recognized by a central hydrophobic cavity and is anchored by a hydrogen bond with an asparagine residue present in most bromodomains⁵. The bromodomain and extra-terminal (BET)-family (BRD2, BRD3, BRD4 and BRDT) shares a common domain architecture comprising two N-terminal bromodomains that exhibit high level of sequence conservation, and a more divergent C-terminal recruitment domain⁶.

The invention features compositions and methods that are useful for inhibiting human bromodomain proteins.

### COMPOUNDS OF THE INVENTION

The invention provides compounds (e.g., JQ1 and compounds of formulas delineated herein) that bind in the binding pocket of the apo crystal structure of the first bromodomain of a BET family member (e.g., BRD2, BRD3, BRD4). The invention provides for the use of such compounds as well as other BRD2, BRD3, and BRD4 inhibitors known in the art in the methods described herein. Such compounds are described, for example, in WO2009084693 and corresponding US2010286127, which is hereby incorporated by reference. Without wishing to be bound by theory, these compounds may be particularly effective in inhibiting adipogenesis, adipocyte differentiation, and deleterious aspects of adipocyte biological activity (e.g., excessive fat synthesis, excessive fat accumulation/adipocyte hypertrophy, adipocyte inflammation, organ fibrosis. In one approach, compounds useful for the treatment of metabolic syndrome, obesity, type II diabetes, insulin resistance, and related disorders characterized by undesirable alterations in metabolism or fat accumulation are selected using a molecular docking program to identify compounds that are expected to bind to a bromodomain structural binding pocket. In certain embodiments, a compound of the invention can prevent, inhibit, or disrupt, or reduce by at least 10%, 25%, 50%, 75%, or 100% the biological activity of a BET family member (e.g., BRD2, BRD3, BRD4, BRDT) and/or disrupt the subcellular localization of such proteins, e.g., by binding to a binding site in a bromodomain apo binding pocket.

In certain embodiments, a compound of the invention is a small molecule having a molecular weight less than about 1000 daltons, less than 800, less than 600, less than 500, less than 400, or less than about 300 daltons. Examples of compounds of the invention include JQ1 and other compounds that bind the binding pocket of the apo crystal structure of the first bromodomain of a BET family member (e.g., BRD4 (hereafter referred to as BRD4(1); PDB ID 2OSS). JQ1 is a novel thieno-triazolo-1,4-diazepine. The invention further provides pharmaceutically acceptable salts of such compounds.

In one aspect, the compound is a compound of Formula I:

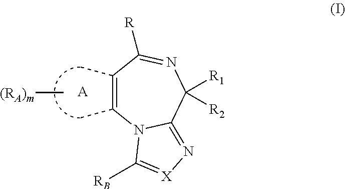

wherein
X is N or CR₅;
   R₅ is H, alkyl, cycloalkyl, heterocycloalkyl, aryl, or heteroaryl, each of which is optionally substituted;
   R_{B} is H, alkyl, hydroxylalkyl, aminoalkyl, alkoxyalkyl, haloalkyl, hydroxy, alkoxy, or —COO—R₃, each of which is optionally substituted;
ring A is aryl or heteroaryl;
   each R_{A} is independently alkyl, cycloalkyl, heterocycloalkyl, aryl, or heteroaryl, each of which is optionally substituted; or any two R_{A} together with the atoms to which each is attached, can form a fused aryl or heteroaryl group;
R is alkyl, cycloalkyl, heterocycloalkyl, aryl, or heteroaryl; each of which is optionally substituted;
R₁ is —(CH₂)ₙ-L, in which n is 0-3 and L is H, —COO—R₃, —CO—R₃, —CO—N(R₃R₄), —S(O)₂—R₃, —S(O)₂—N(R₃R₄), N(R₃R₄), N(R₄)C(O)R₃, optionally substituted aryl, or optionally substituted heteroaryl;
R₂ is H, D (deuterium), halogen, or optionally substituted alkyl;
each R₃ is independently selected from the group consisting of:
   (i) H, aryl, substituted aryl, heteroaryl, or substituted heteroaryl;
   (ii) heterocycloalkyl or substituted heterocycloalkyl;
   (iii) —C₁-C₈ alkyl, —C₂-C₈ alkenyl or —C₂-C₈ alkynyl, each containing 0, 1, 2, or 3 heteroatoms selected from O, S, or N; —C₃-C₁₂ cycloalkyl, substituted —C₃-C₁₂ cycloalkyl, —C₃-C₁₂ cycloalkenyl, or substituted —C₃-C₁₂ cycloalkenyl, each of which may be optionally substituted; and
   (iv) NH₂, N═CR₄R₆;
   each R₄ is independently H, alkyl, alkyl, cycloalkyl, heterocycloalkyl, aryl, or heteroaryl, each of which is optionally substituted;
   or R₃ and R₄ are taken together with the nitrogen atom to which they are attached to form a 4-10-membered ring;
   R₆ is alkyl, alkenyl, cycloalkyl, cycloalkenyl, heterocycloalkyl, aryl, or heteroaryl, each of which is optionally substituted; or R₄ and R₆ are taken together with the carbon atom to which they are attached to form a 4-10-membered ring;
m is 0, 1, 2, or 3;
provided that
   (a) if ring A is thienyl, X is N, R is phenyl or substituted phenyl, R₂ is H, R_{B} is methyl, and R₁ is —(CH₂)ₙ-L, in which n is 1 and L is —CO—N(R₃R₄), then R₃ and R₄ are not taken together with the nitrogen atom to which they are attached to form a morpholino ring;
   (b) if ring A is thienyl, X is N, R is substituted phenyl, R₂ is H, R_{B} is methyl, and R₁ is —(CH₂)ₙ-L, in which n is 1 and L is —CO—N(R₃R₄), and one of R₃ and R₄ is H, then the other of R₃ and R₄ is not methyl, hydroxyethyl, alkoxy, phenyl, substituted phenyl, pyridyl or substituted pyridyl; and
   (c) if ring A is thienyl, X is N, R is substituted phenyl, R₂ is H, R_{B} is methyl, and R₁ is —(CH₂)ₙ-L, in which n is 1 and L is —COO—R₃, then R₃ is not methyl or ethyl;
   or a salt, solvate or hydrate thereof.

In certain embodiments, R is aryl or heteroaryl, each of which is optionally substituted.

In certain embodiments, L is H, —COO—R₃, —CO—N(R₃R₄), —S(O)₂—R₃, —S(O)₂—N(R₃R₄), N(R₃R₄), N(R₄)C(O)R₃ or optionally substituted aryl. In certain embodiments, each R₃ is independently selected from the group consisting of: H, —C₁-C₈ alkyl, containing 0, 1, 2, or 3 heteroatoms selected from O, S, or N; or NH₂, N═CR₄R₆.

In certain embodiments, R₂ is H, D, halogen or methyl.

In certain embodiments, R_{B} is alkyl, hydroxyalkyl, haloalkyl, or alkoxy; each of which is optionally substituted.

In certain embodiments, R_{B} is methyl, ethyl, hydroxy methyl, methoxymethyl, trifluoromethyl, COOH, COOMe, COOEt, or COOCH₂OC(O)CH₃.

In certain embodiments, ring A is a 5 or 6-membered aryl or heteroaryl. In certain embodiments, ring A is thiofuranyl, phenyl, naphthyl, biphenyl, tetrahydronaphthyl, indanyl, pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, or 5,6,7,8-tetrahydroisoquinolinyl.

In certain embodiments, ring A is phenyl or thienyl.

In certain embodiments, m is 1 or 2, and at least one occurrence of R_{A} is methyl.

In certain embodiments, each R_{A} is independently H, an optionally substituted alkyl, or any two R_{A} together with the atoms to which each is attached, can form an aryl.

In another aspect, the compound is a compound of Formula II:

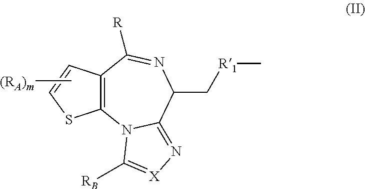

wherein
X is N or CR₅;
   R₅ is H, alkyl, cycloalkyl, heterocycloalkyl, aryl, or heteroaryl, each of which is optionally substituted;
   R_{B} is H, alkyl, hydroxylalkyl, aminoalkyl, alkoxyalkyl, haloalkyl, hydroxy, alkoxy, or —COO—R₃, each of which is optionally substituted;
each R_{A} is independently alkyl, cycloalkyl, heterocycloalkyl, aryl, or heteroaryl, each of which is optionally substituted; or any two R_{A} together with the atoms to which each is attached, can form a fused aryl or heteroaryl group;
R is alkyl, cycloalkyl, heterocycloalkyl, aryl, or heteroaryl, each of which is optionally substituted;
R′₁ is H, —COO—R₃, —CO—R₃, optionally substituted aryl, or optionally substituted heteroaryl;
   each R₃ is independently selected from the group consisting of:
   (i) H, aryl, substituted aryl, heteroaryl, substituted heteroaryl;
   (ii) heterocycloalkyl or substituted heterocycloalkyl;
   (iii) —C₁-C₈ alkyl, —C₂-C₈ alkenyl or —C₂-C₈ alkynyl, each containing 0, 1, 2, or 3 heteroatoms selected from O, S, or N; —C₃-C₁₂ cycloalkyl, substituted —C₃-C₁₂ cycloalkyl; —C₃-C₁₂ cycloalkenyl, or substituted —C₃-C₁₂ cycloalkenyl; each of which may be optionally substituted;
m is 0, 1, 2, or 3;
provided that if R′₁ is —COO—R₃, X is N, R is substituted phenyl, and R_{B} is methyl, then R₃ is not methyl or ethyl;
or a salt, solvate or hydrate thereof.

In certain embodiments, R is aryl or heteroaryl, each of which is optionally substituted. In certain embodiments, R is phenyl or pyridyl, each of which is optionally substituted. In certain embodiments, R is p-Cl-phenyl, o-Cl-phenyl, m-Cl-phenyl, p-F-phenyl, o-F-phenyl, m-F-phenyl or pyridinyl.

In certain embodiments, R′₁ is —COO—R₃, optionally substituted aryl, or optionally substituted heteroaryl; and R₃ is —C₁-C₈ alkyl, which contains 0, 1, 2, or 3 heteroatoms selected from O, S, or N, and which may be optionally substituted. In certain embodiments, R′₁ is —COO—R₃, and R₃ is methyl, ethyl, propyl, i-propyl, butyl, sec-butyl, or t-butyl; or R′₁ is H or optionally substituted phenyl.

In certain embodiments, R_{B} is methyl, ethyl, hydroxy methyl, methoxymethyl, trifluoromethyl, COOH, COOMe, COOEt, COOCH₂OC(O)CH₃.

In certain embodiments, R_{B} is methyl, ethyl, hydroxy methyl, methoxymethyl, trifluoromethyl, COOH, COOMe, COOEt, or COOCH₂OC(O)CH₃.

In certain embodiments, each R_{A} is independently an optionally substituted alkyl, or any two R_{A} together with the atoms to which each is attached, can form a fused aryl.

In certain embodiments, each R_{A} is methyl.

In another aspect, the compound is a compound of formula III:

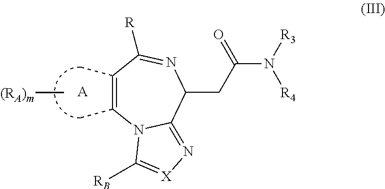

wherein
X is N or CR₅;
   R₅ is H, alkyl, cycloalkyl, heterocycloalkyl, aryl, or heteroaryl, each of which is optionally substituted;
   R_{B} is H, alkyl, hydroxylalkyl, aminoalkyl, alkoxyalkyl, haloalkyl, hydroxy, alkoxy, or —COO—R₃, each of which is optionally substituted;
ring A is aryl or heteroaryl;
   each R_{A} is independently alkyl, cycloalkyl, heterocycloalkyl, aryl, or heteroaryl, each of which is optionally substituted; or any two R_{A} together with the atoms to which each is attached, can form a fused aryl or heteroaryl group;
R is alkyl, cycloalkyl, heterocycloalkyl, aryl, or heteroaryl, each of which is optionally substituted;
each R₃ is independently selected from the group consisting of:
   (i) H, aryl, substituted aryl, heteroaryl, or substituted heteroaryl;
   (ii) heterocycloalkyl or substituted heterocycloalkyl;
   (iii) —C₁-C₈ alkyl, —C₂-C₈ alkenyl or —C₂-C₈ alkynyl, each containing 0, 1, 2, or 3 heteroatoms selected from O, S, or N; —C₃-C₁₂ cycloalkyl, substituted —C₃-C₁₂ cycloalkyl, —C₃-C₁₂ cycloalkenyl, or substituted —C₃-C₁₂ cycloalkenyl, each of which may be optionally substituted; and
   (iv) NH₂, N═CR₄R₆;
   each R₄ is independently H, alkyl, alkyl, cycloalkyl, heterocycloalkyl, aryl, or heteroaryl, each of which is optionally substituted;
   or R₃ and R₄ are taken together with the nitrogen atom to which they are attached to form a 4-10-membered ring;
   R₆ is alkyl, alkenyl, cycloalkyl, cycloalkenyl, heterocycloalkyl, aryl, or heteroaryl, each of which is optionally substituted; or R₄ and R₆ are taken together with the carbon atom to which they are attached to form a 4-10-membered ring;
m is 0, 1, 2, or 3;
provided that:
   (a) if ring A is thienyl, X is N, R is phenyl or substituted phenyl, R_{B} is methyl, then R₃ and R₄ are not taken together with the nitrogen atom to which they are attached to form a morpholino ring; and
   (b) if ring A is thienyl, X is N, R is substituted phenyl, R₂ is H, R_{B} is methyl, and one of R₃ and R₄ is H, then the other of R₃ and R₄ is not methyl, hydroxyethyl, alkoxy, phenyl, substituted phenyl, pyridyl or substituted pyridyl; and
or a salt, solvate or hydrate thereof.

In certain embodiments, R is aryl or heteroaryl, each of which is optionally substituted. In certain embodiments, R is phenyl or pyridyl, each of which is optionally substituted.

In certain embodiments, R is p-Cl-phenyl, o-Cl-phenyl, m-Cl-phenyl, p-F-phenyl, o-F-phenyl, m-F-phenyl or pyridinyl. In certain embodiments, R₃ is H, NH₂, or N═CR₄R₆.

In certain embodiments, each R₄ is independently H, alkyl, cycloalkyl, heterocycloalkyl, aryl, heteroaryl; each of which is optionally substituted.

In certain embodiments, R₆ is alkyl, alkenyl, cycloalkyl, cycloalkenyl, heterocycloalkyl, aryl, or heteroaryl, each of which is optionally substituted.

In another aspect, the compound is a compound of formula IV:

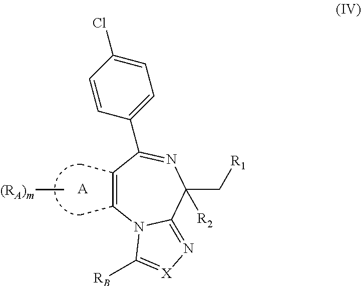

wherein
X is N or CR₅;
   R₅ is H, alkyl, cycloalkyl, heterocycloalkyl, aryl, or heteroaryl, each of which is optionally substituted;
   R_{B} is H, alkyl, hydroxylalkyl, aminoalkyl, alkoxyalkyl, haloalkyl, hydroxy, alkoxy, or —COO—R₃, each of which is optionally substituted;
ring A is aryl or heteroaryl;
   each R_{A} is independently alkyl, cycloalkyl, heterocycloalkyl, aryl, or heteroaryl, each of which is optionally substituted; or any two R_{A} together with the atoms to which each is attached, can form a fused aryl or heteroaryl group;
R₁ is —(CH₂)ₙ-L, in which n is 0-3 and L is H, —COO—R₃, —CO—R₃, —CO—N(R₃R₄), —S(O)₂—R₃, —S(O)₂—N(R₃R₄), N(R₃R₄), N(R₄)C(O)R₃, optionally substituted aryl, or optionally substituted heteroaryl;
R₂ is H, D, halogen, or optionally substituted alkyl;
each R₃ is independently selected from the group consisting of:
   (i) H, aryl, substituted aryl, heteroaryl, or substituted heteroaryl;
   (ii) heterocycloalkyl or substituted heterocycloalkyl;
   (iii) —C₁-C₈ alkyl, —C₂-C₈ alkenyl or —C₂-C₈ alkynyl, each containing 0, 1, 2, or 3 heteroatoms selected from O, S, or N; —C₃-C₁₂ cycloalkyl, substituted —C₃-C₁₂ cycloalkyl, —C₃-C₁₂ cycloalkenyl, or substituted —C₃-C₁₂ cycloalkenyl, each of which may be optionally substituted; and
   (iv) NH₂, N═CR₄R₆;
each R₄ is independently H, alkyl, alkyl, cycloalkyl, heterocycloalkyl, aryl, or heteroaryl, each of which is optionally substituted;
or R₃ and R₄ are taken together with the nitrogen atom to which they are attached to form a 4-10-membered ring;
R₆ is alkyl, alkenyl, cycloalkyl, cycloalkenyl, heterocycloalkyl, aryl, or heteroaryl, each of which is optionally substituted; or R₄ and R₆ are taken together with the carbon atom to which they are attached to form a 4-10-membered ring;
m is 0, 1, 2, or 3;
provided that
   (a) if ring A is thienyl, X is N, R₂ is H, R_{B} is methyl, and R₁ is —(CH₂)ₙ-L, in which n is 0 and L is —CO—N(R₃R₄), then R₃ and R₄ are not taken together with the nitrogen atom to which they are attached to form a morpholino ring;
   (b) if ring A is thienyl, X is N, R₂ is H, R_{B} is methyl, and R₁ is —(CH₂)ₙ-L, in which n is 0 and L is —CO—N(R₃R₄), and one of R₃ and R₄ is H, then the other of R₃ and R₄ is not methyl, hydroxyethyl, alkoxy, phenyl, substituted phenyl, pyridyl or substituted pyridyl; and
   (c) if ring A is thienyl, X is N, R₂ is H, R_{B} is methyl, and R₁ is —(CH₂)ₙ-L, in which n is 0 and L is —COO—R₃, then R₃ is not methyl or ethyl; or
      a salt, solvate or hydrate thereof.

In certain embodiments, R₁ is —(CH₂)ₙ-L, in which n is 0-3 and L is —COO—R₃, optionally substituted aryl, or optionally substituted heteroaryl; and R₃ is —C₁-C₈ alkyl, which contains 0, 1, 2, or 3 heteroatoms selected from O, S, or N, and which may be optionally substituted. In certain embodiments, n is 1 or 2 and L is alkyl or —COO—R₃, and R₃ is methyl, ethyl, propyl, i-propyl, butyl, sec-butyl, or t-butyl; or n is 1 or 2 and L is H or optionally substituted phenyl.

In certain embodiments, R₂ is H or methyl.

In certain embodiments, R_{B} is methyl, ethyl, hydroxy methyl, methoxymethyl, trifluoromethyl, COOH, COOMe, COOEt, COOCH₂OC(O)CH₃.

In certain embodiments, ring A is phenyl, naphthyl, biphenyl, tetrahydronaphthyl, indanyl, pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, or 5,6,7,8-tetrahydroisoquinolinyl.

In certain embodiments, each R_{A} is independently an optionally substituted alkyl, or any two R_{A} together with the atoms to which each is attached, can form an aryl.

The methods of the invention also relate to compounds of Formulae V-XXII, and to any compound described herein.

In another aspect, the compound is a compound represented by the formula:

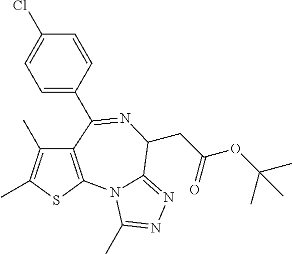 [SMILES: Cc1sc2c(c1C)C(c1ccc(Cl)cc1)=NC(CC(=O)OC(C)(C)C)c1nnc(C)n1-2]

or a salt, solvate or hydrate thereof.

In certain embodiments, the compound is (+)-JQ1:

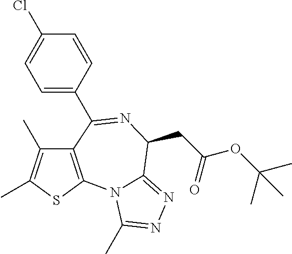 [SMILES: Cc1sc2c(c1C)C(c1ccc(Cl)cc1)=N[C@@H](CC(=O)OC(C)(C)C)c1nnc(C)n1-2]

or a salt, solvate or hydrate thereof.

In another aspect, the compound is a compound represented by the formula:

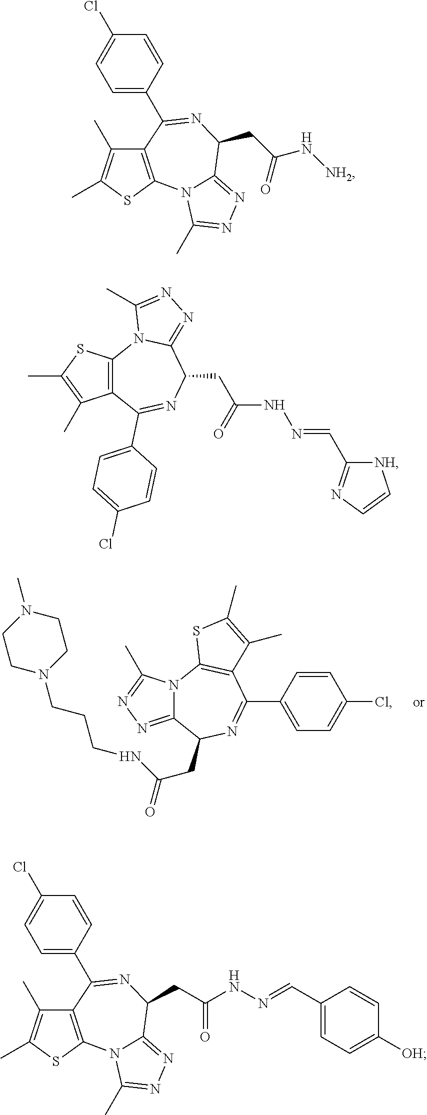

or a salt, solvate or hydrate thereof.

In another aspect, the compound is a compound represented by the formula:

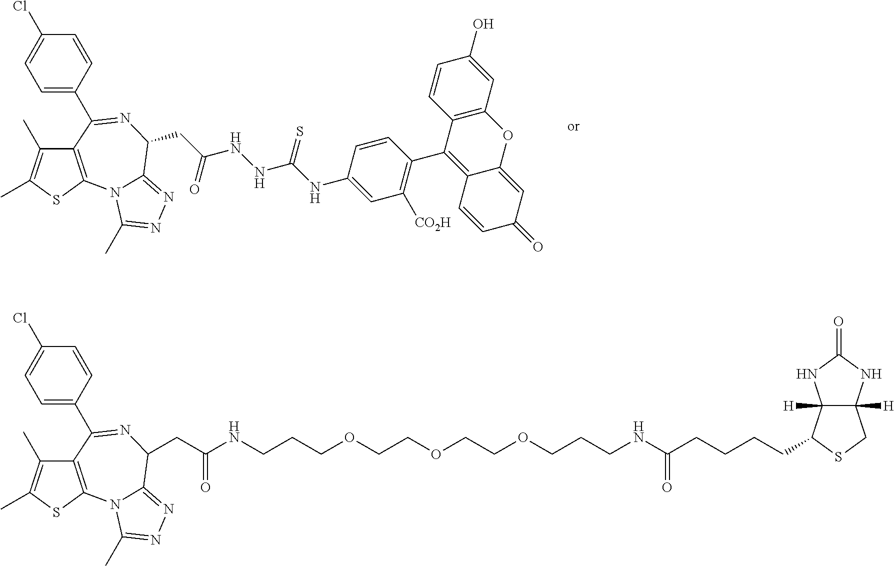 [SMILES: Cc1sc2c(c1C)C(c1ccc(Cl)cc1)=N[C@H](CC(=O)NNC(=S)Nc1ccc(-c3c4ccc(=O)cc-4oc4cc(O)ccc34)c(C(=O)O)c1)c1nnc(C)n1-2 ; Cc1sc2c(c1C)C(c1ccc(Cl)cc1)=NC(CC(=O)NCCCOCCOCCOCCCNC(=O)CCCC[C@H]1SC[C@H]3NC(=O)N[C@H]31)c1nnc(C)n1-2]

or a salt, solvate or hydrate thereof.

In another aspect, the compound is a compound represented by any one of the following formulae:

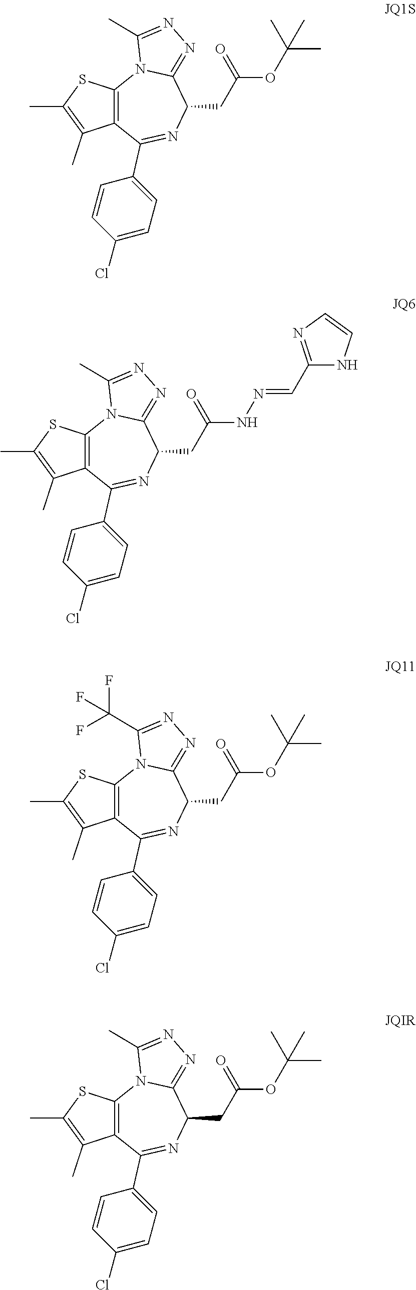

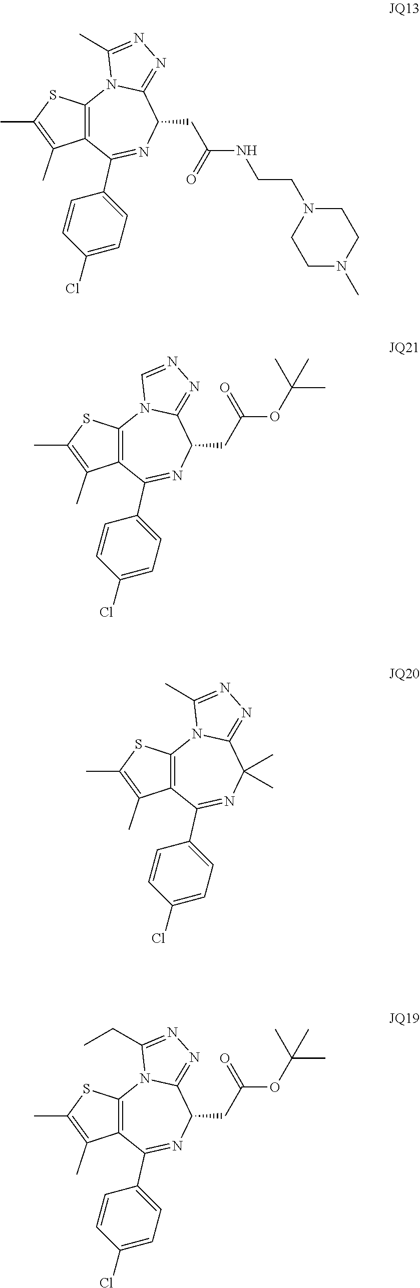

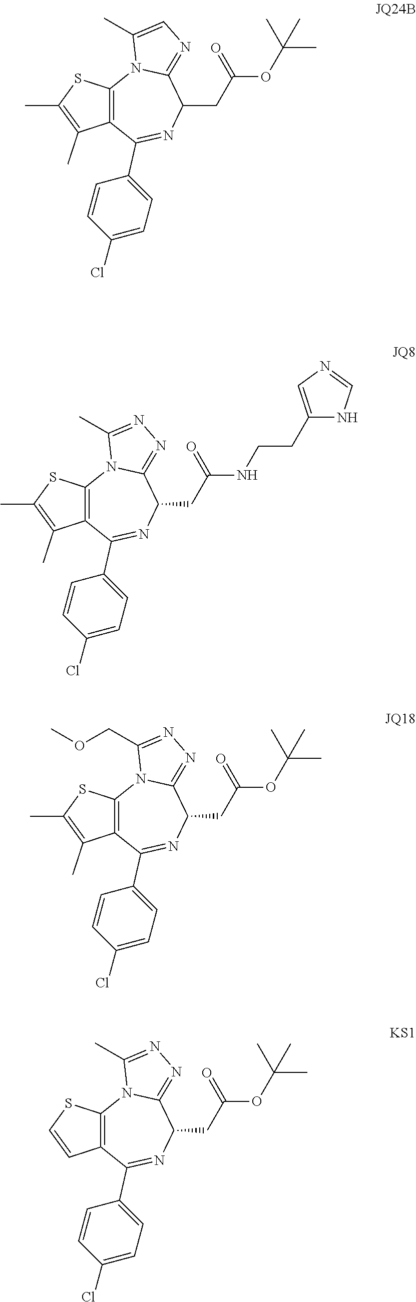

or a salt, solvate or hydrate thereof.

In another aspect, the compound is a compound represented by any one of the following formulae:

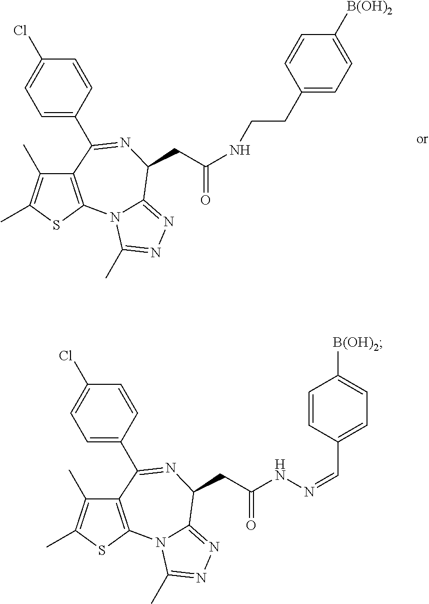 [SMILES: Cc1sc2c(c1C)C(c1ccc(Cl)cc1)=N[C@@H](CC(=O)NCCc1ccc(B(O)O)cc1)c1nnc(C)n1-2 ; Cc1sc2c(c1C)C(c1ccc(Cl)cc1)=N[C@@H](CC(=O)N/N=C\c1ccc(B(O)O)cc1)c1nnc(C)n1-2]

or a salt, solvate or hydrate thereof.

In another aspect, the compound is a compound represented by any one of the following structures:

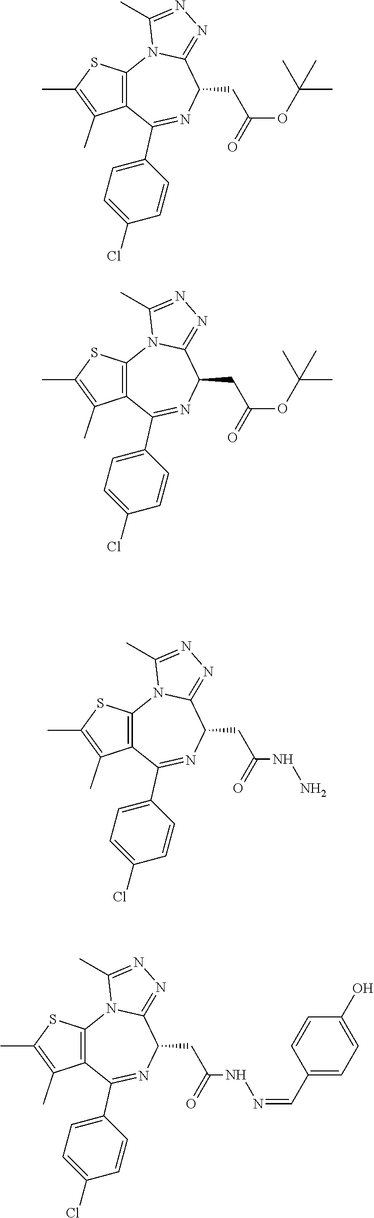

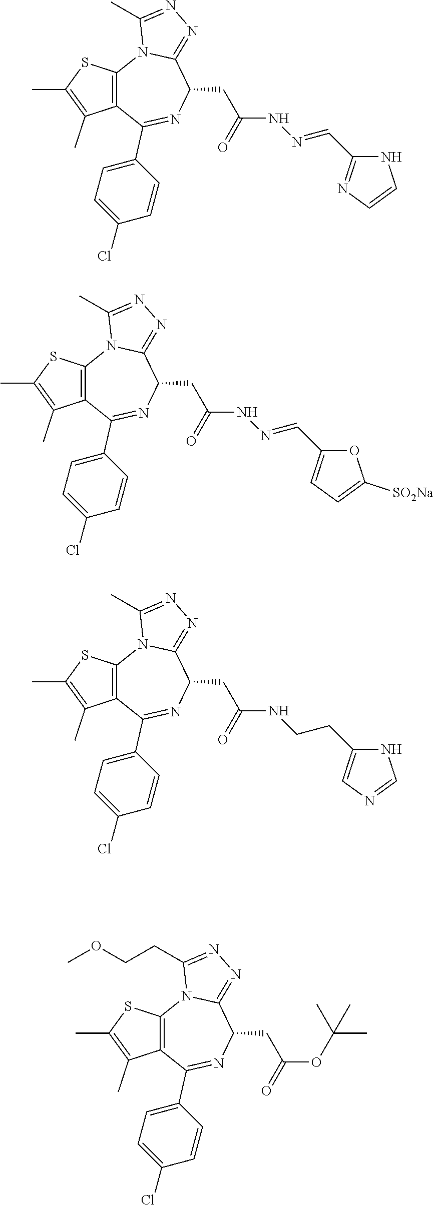

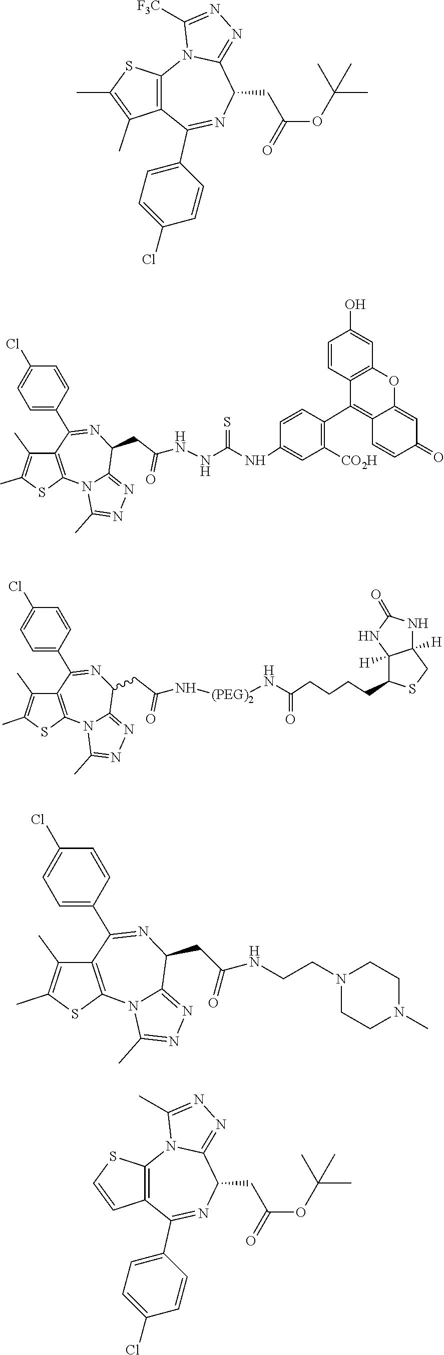

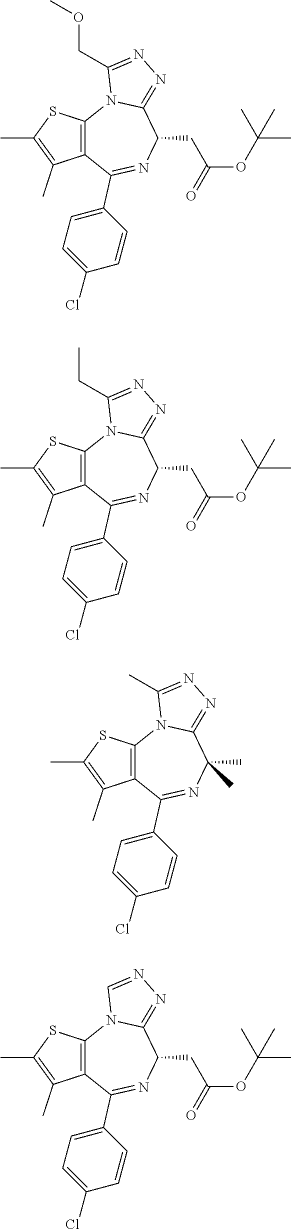

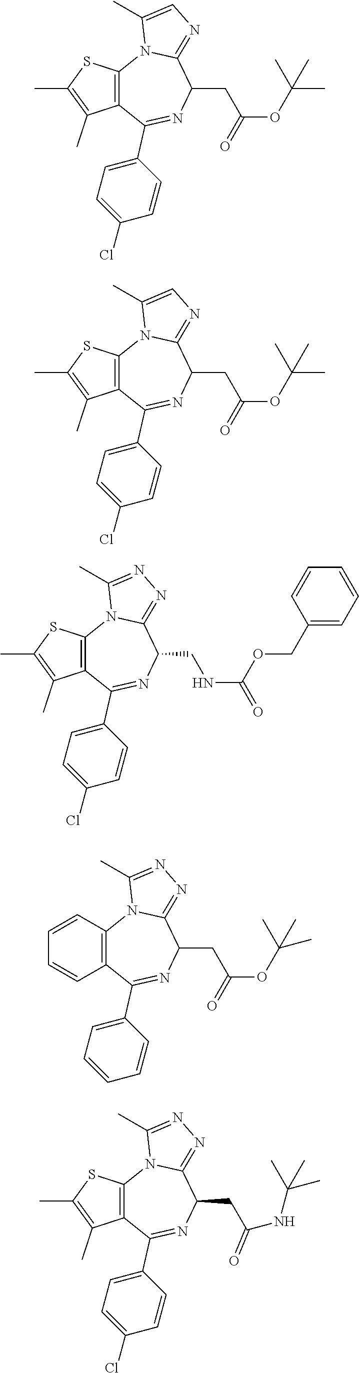

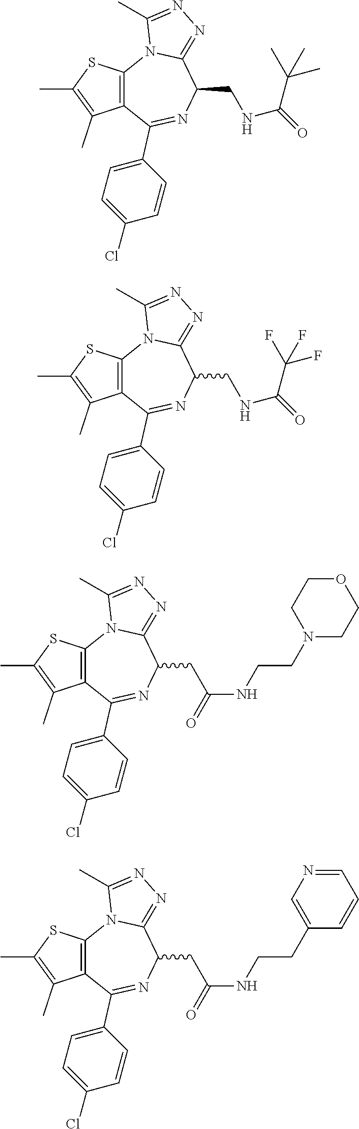

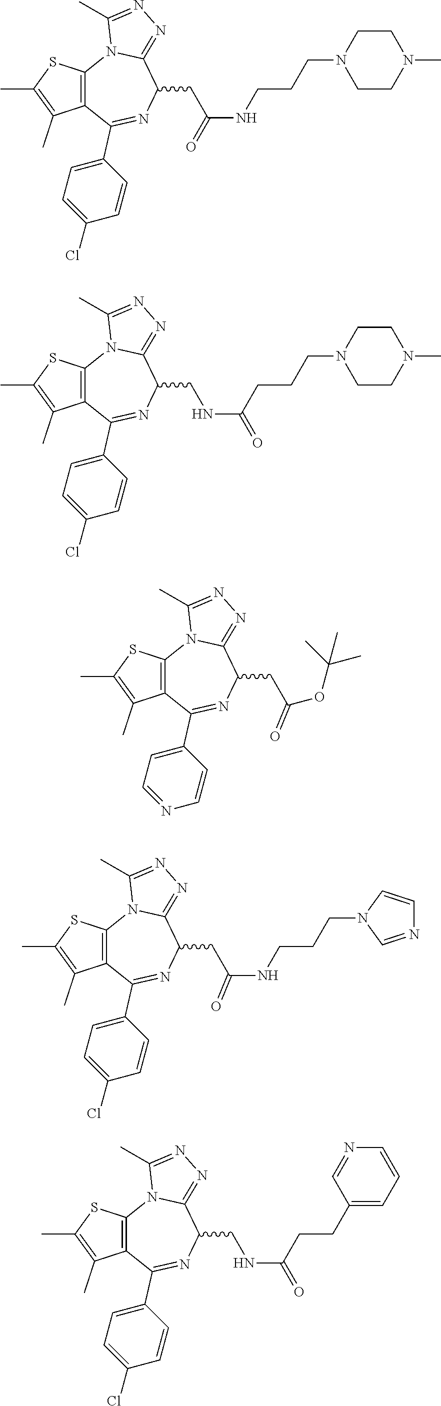

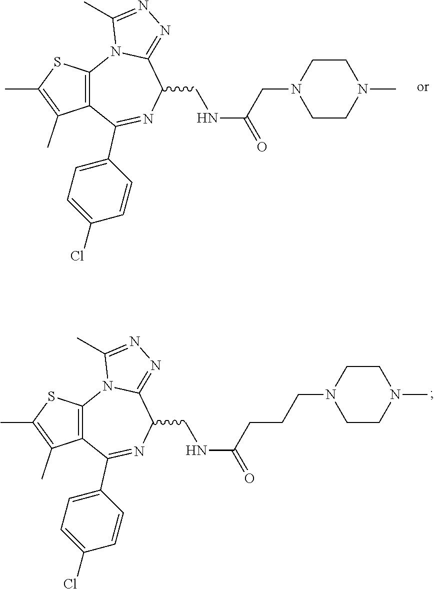

or a salt, solvate or hydrate thereof.

In certain embodiments, a compound of the invention can be represented by one of the following structures:

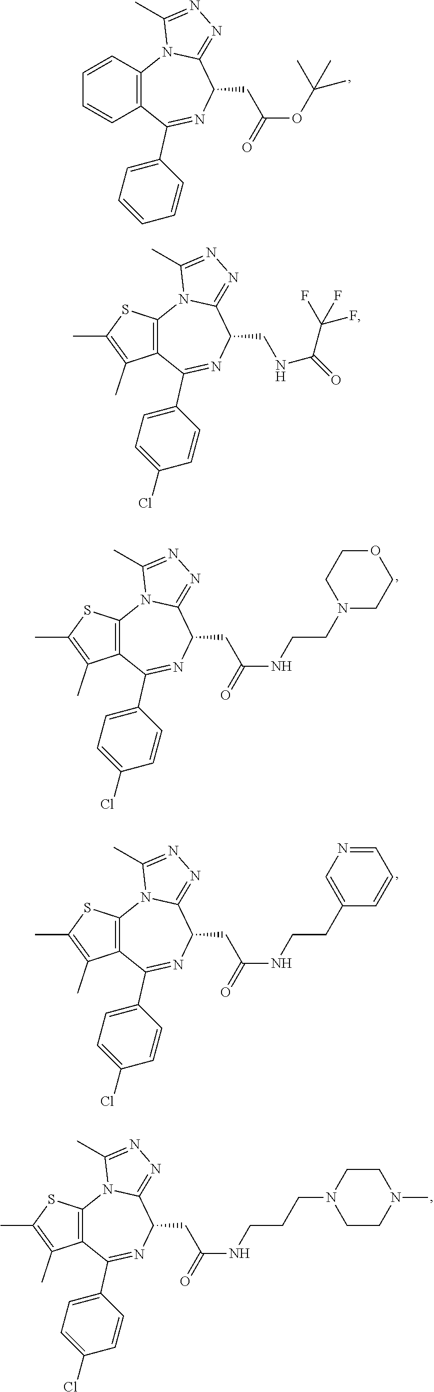

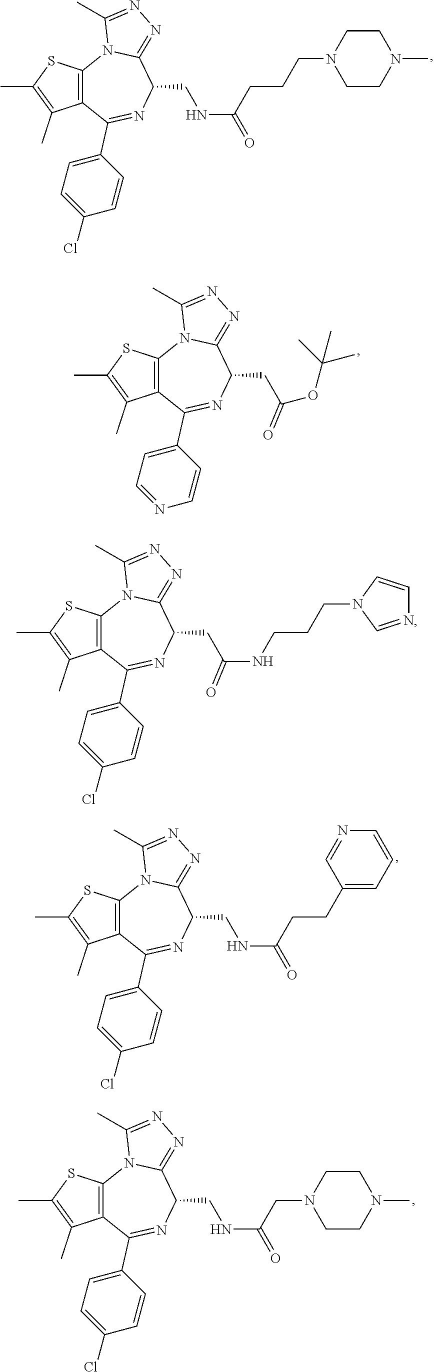

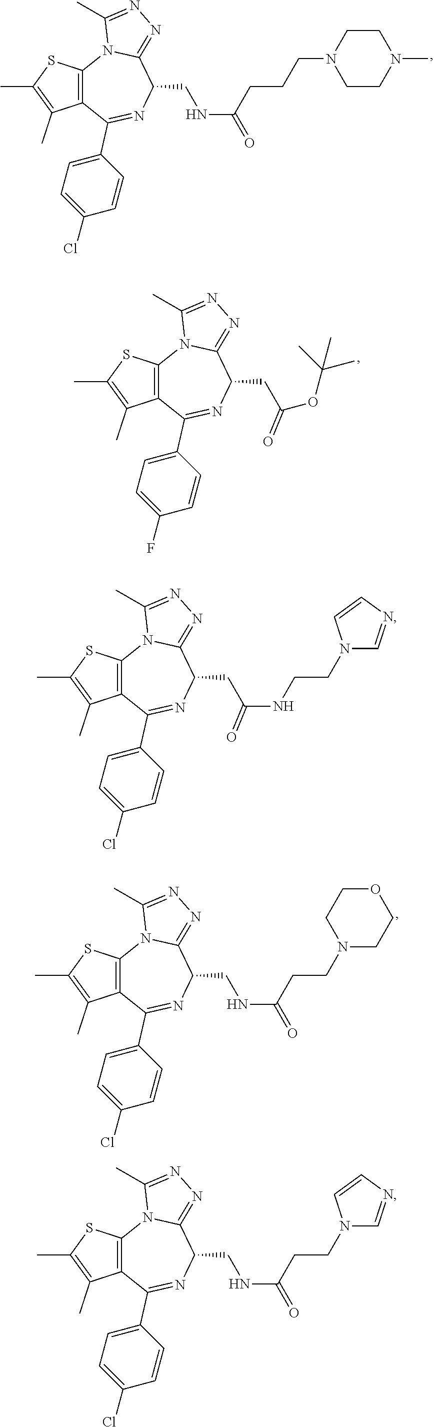

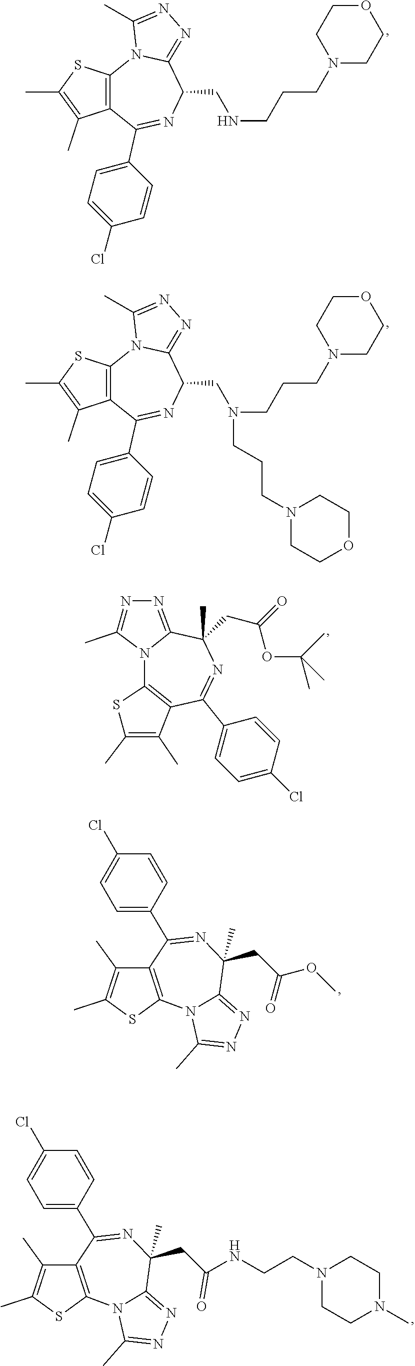

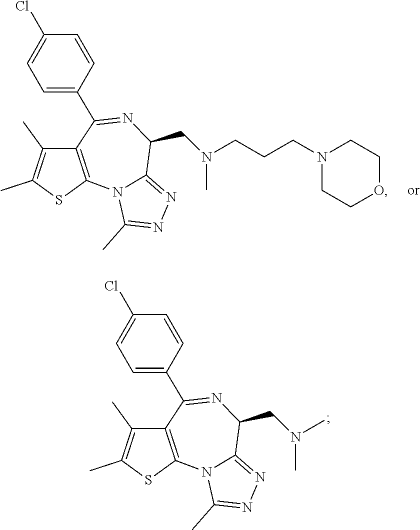

or a salt, solvate or hydrate thereof.

In one embodiment, the compound is represented by the structure:

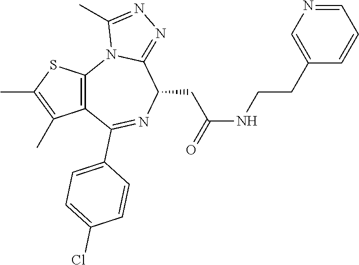 [SMILES: Cc1sc2c(c1C)C(c1ccc(Cl)cc1)=N[C@@H](CC(=O)NCCc1cccnc1)c1nnc(C)n1-2]

or a salt, solvate or hydrate thereof.

In another embodiment, the compound is represented by the structure:

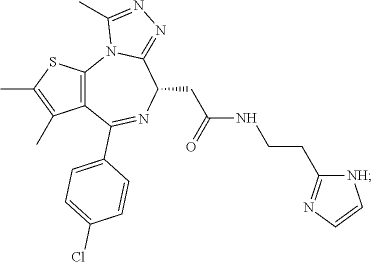 [SMILES: Cc1sc2c(c1C)C(c1ccc(Cl)cc1)=N[C@@H](CC(=O)NCCc1ncc[nH]1)c1nnc(C)n1-2]

or a salt, solvate or hydrate thereof.

In another embodiment, the compound is represented by the structure:

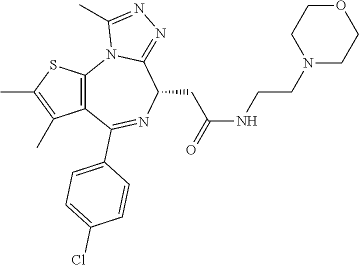 [SMILES: Cc1sc2c(c1C)C(c1ccc(Cl)cc1)=N[C@@H](CC(=O)NCCN1CCOCC1)c1nnc(C)n1-2]

or a salt, solvate or hydrate thereof.

In certain embodiments, a compound of the invention can have the opposite chirality of any compound shown herein.

In certain embodiments, the compound is a compound represented by Formula (V), (VI), or (VII):

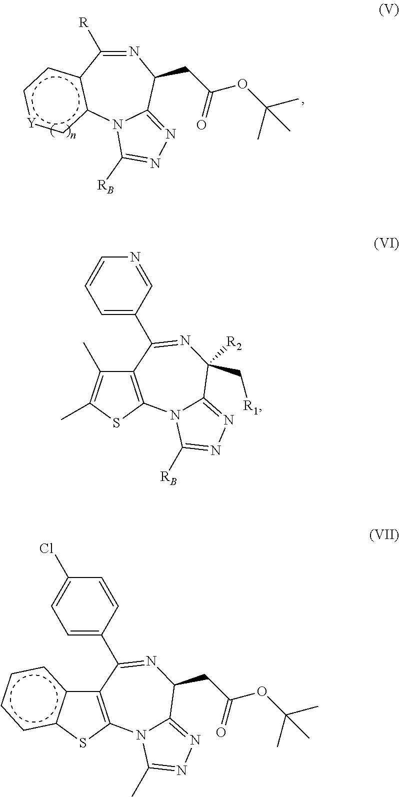

in which R, R₁, and R₂ and R_{B} have the same meaning as in Formula (I); Y is O, N, S, or CR₅, in which R₅ has the same meaning as in Formula (I); n is 0 or 1; and the dashed circle in Formula (VII) indicates an aromatic or non-aromatic ring; or a salt, solvate, or hydrate thereof.

In certain embodiments of any of the Formulae I-IV and VI (or any formula herein), R₆ represents the non-carbonyl portion of an aldehyde shown in Table A, below (i.e., for an aldehyde of formula R₆CHO, R₆ is the non-carbonyl portion of the aldehyde). In certain embodiments, R₄ and R₆ together represent the non-carbonyl portion of a ketone shown in Table A (i.e., for a ketone of formula R₆C(O)R₄, R₄ and R₆ are the non-carbonyl portion of the ketone).

TABLE A
Plate 1

| | 01 | 02 | 03 |
|---|---|---|---|
| A | | 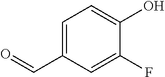 | 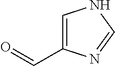 |
| B | 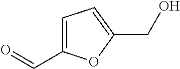 | 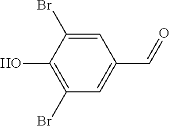 [SMILES: O=Cc1cc(Br)c(O)c(Br)c1] | 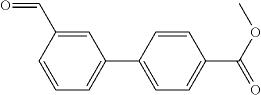 [SMILES: COC(=O)c1ccc(-c2cccc(C=O)c2)cc1] |
| C | 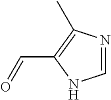 | 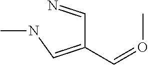 | 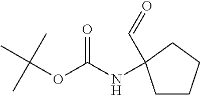 |
| D | 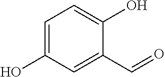 | 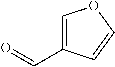 | 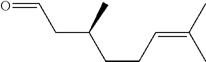 |
| E | 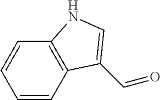 [SMILES: O=Cc1c[nH]c2ccccc12] | 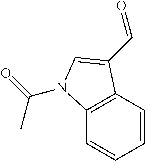 | 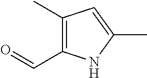 |
| F | 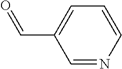 | 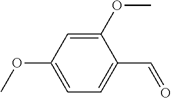 | 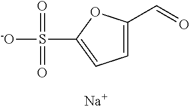 |
| G | 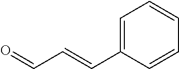 | 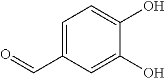 | 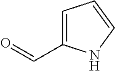 |
| H | 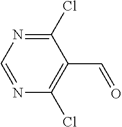 | 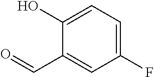 | 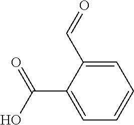 [SMILES: O=Cc1ccccc1C(=O)O] |

Plate 2

| | 01 | 02 | 03 |
|---|---|---|---|
| A | | 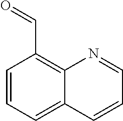 [SMILES: O=Cc1cccc2cccnc12] | 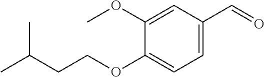 [SMILES: COc1cc(C=O)ccc1OCCC(C)C] |
| B | 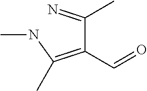 | 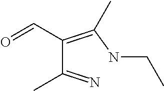 | 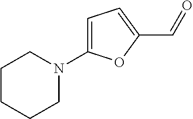 [SMILES: O=Cc1ccc(N2CCCCC2)o1] |
| C | 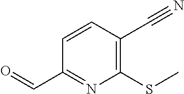 | 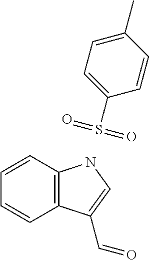 | 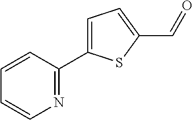 [SMILES: O=Cc1ccc(-c2ccccn2)s1] |
| D | 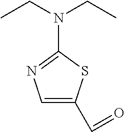 | 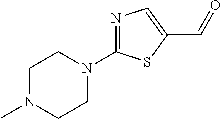 | 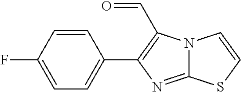 [SMILES: O=Cc1c(-c2ccc(F)cc2)nc2sccn12] |
| E | 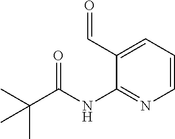 | 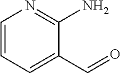 | 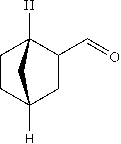 |
| F | 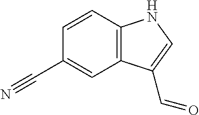 [SMILES: N#Cc1ccc2[nH]cc(C=O)c2c1] | 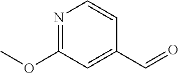 | 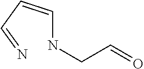 |
| G | 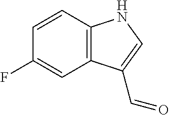 [SMILES: O=Cc1c[nH]c2ccc(F)cc12] | 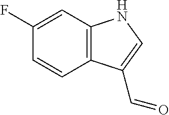 [SMILES: O=Cc1c[nH]c2cc(F)ccc12] | 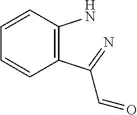 |
| H | 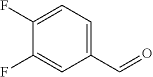 | 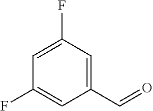 | 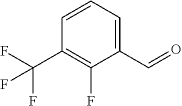 [SMILES: O=Cc1cccc(C(F)(F)F)c1F] |

Plate 3

| | 01 | 02 | 03 |
|---|---|---|---|
| A | | 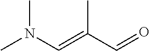 | 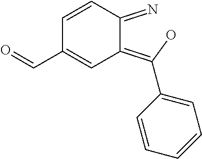 [SMILES: N=C1C=CC(C=O)=C/C1=C(/O)c1ccccc1] |
| B | 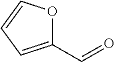 | 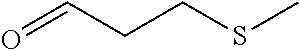 | 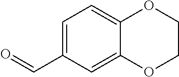 [SMILES: O=Cc1ccc2c(c1)OCCO2] |
| C | 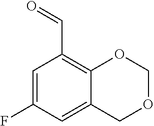 [SMILES: O=Cc1cc(F)cc2c1OCOC2] | 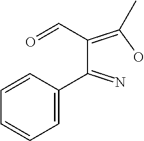 | 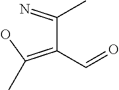 |
| D | 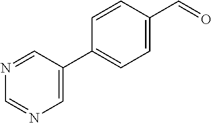 [SMILES: O=Cc1ccc(-c2cncnc2)cc1] | 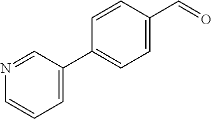 [SMILES: O=Cc1ccc(-c2cccnc2)cc1] | 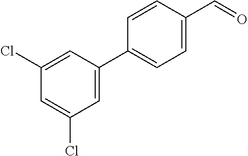 [SMILES: O=Cc1ccc(-c2cc(Cl)cc(Cl)c2)cc1] |
| E | 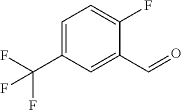 [SMILES: O=Cc1cc(C(F)(F)F)ccc1F] | 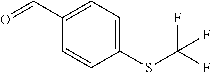 [SMILES: O=Cc1ccc(SC(F)(F)F)cc1] | 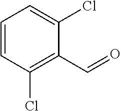 |
| F | 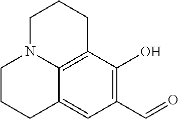 [SMILES: O=Cc1cc2c3c(c1O)CCCN3CCC2] | 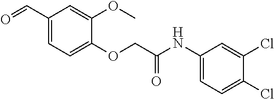 [SMILES: COc1cc(C=O)ccc1OCC(=O)Nc1ccc(Cl)c(Cl)c1] | 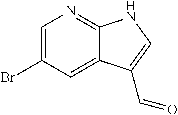 [SMILES: O=Cc1c[nH]c2ncc(Br)cc12] |
| G | 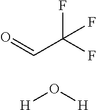 | 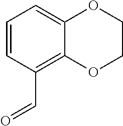 [SMILES: O=Cc1cccc2c1OCCO2] | 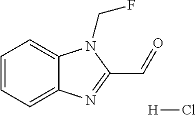 |
| H | 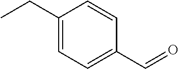 | 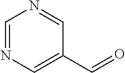 | 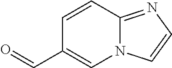 [SMILES: O=Cc1ccc2nccn2c1] |

Plate 4

| | 01 | 02 | 03 |
|---|---|---|---|
| A | | 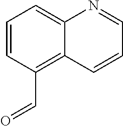 [SMILES: O=Cc1cccc2ncccc12] | 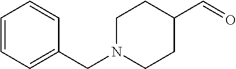 [SMILES: O=CC1CCN(Cc2ccccc2)CC1] |
| B | 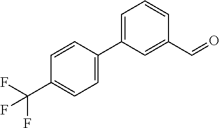 [SMILES: O=Cc1cccc(-c2ccc(C(F)(F)F)cc2)c1] | 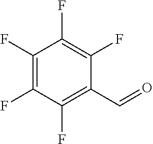 [SMILES: O=Cc1c(F)c(F)c(F)c(F)c1F] | 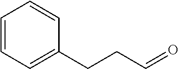 |
| C | 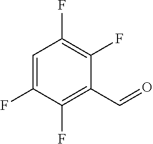 [SMILES: O=Cc1c(F)c(F)cc(F)c1F] | 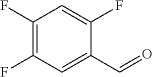 [SMILES: O=Cc1cc(F)c(F)cc1F] | 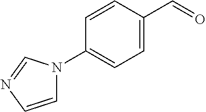 [SMILES: O=Cc1ccc(-n2ccnc2)cc1] |
| D | 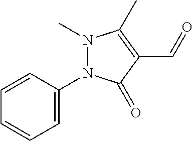 [SMILES: Cc1c(C=O)c(=O)n(-c2ccccc2)n1C] | 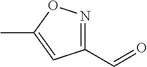 | 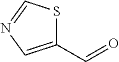 |
| E | 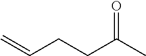 | 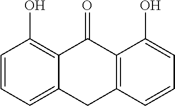 [SMILES: O=C1c2c(O)cccc2Cc2cccc(O)c21] | 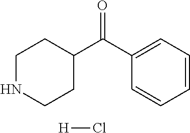 |
| F | 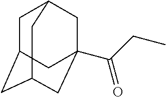 | 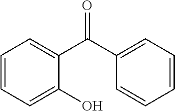 [SMILES: O=C(c1ccccc1)c1ccccc1O] | 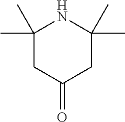 |
| G | 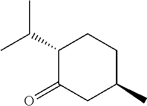 | 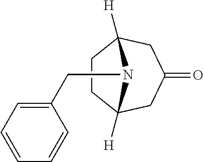 [SMILES: O=C1C[C@H]2CC[C@@H](C1)N2Cc1ccccc1] | 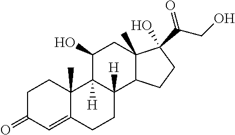 |

Plate 1

| | 04 | 05 | 06 |
|---|---|---|---|
| A | 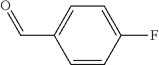 | 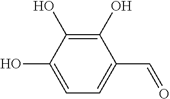 [SMILES: O=Cc1ccc(O)c(O)c1O] | 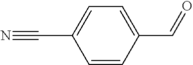 |
| B | 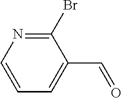 | 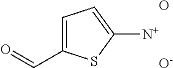 | 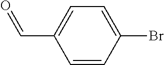 |
| C | 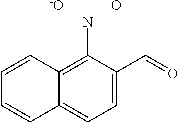 | | 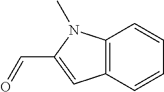 |
| D | 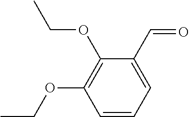 | 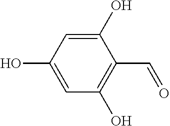 [SMILES: O=Cc1c(O)cc(O)cc1O] | 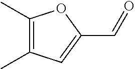 |
| E | 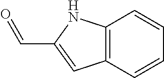 [SMILES: O=Cc1cc2ccccc2[nH]1] | 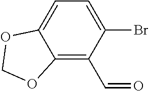 [SMILES: O=Cc1c(Br)ccc2c1OCO2] | 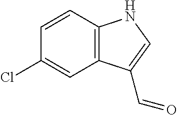 [SMILES: O=Cc1c[nH]c2ccc(Cl)cc12] |
| F | 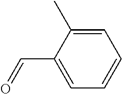 | 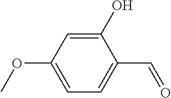 | 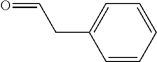 |
| G | 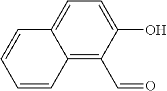 [SMILES: O=Cc1c(O)ccc2ccccc12] | 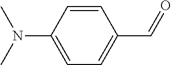 | 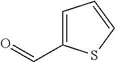 |
| H | 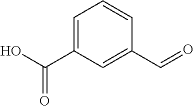 [SMILES: O=Cc1cccc(C(=O)O)c1] | 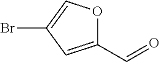 | 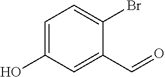 |

Plate 2

| | 04 | 05 | 06 |
|---|---|---|---|
| A | 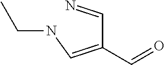 | 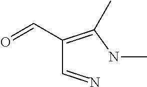 | 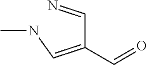 |
| B | 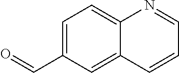 [SMILES: O=Cc1ccc2ncccc2c1] | 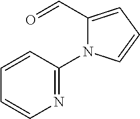 [SMILES: O=Cc1cccn1-c1ccccn1] | 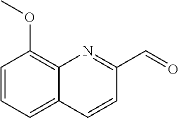 |
| C | 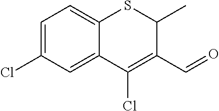 | | 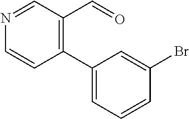 [SMILES: O=Cc1cnccc1-c1cccc(Br)c1] |
| D | 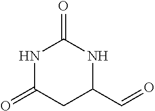 | 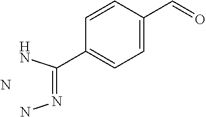 | 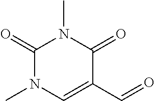 |
| E | 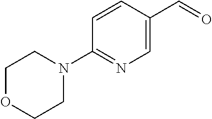 [SMILES: O=Cc1ccc(N2CCOCC2)nc1] | 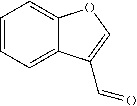 [SMILES: O=Cc1coc2ccccc12] | 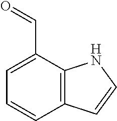 [SMILES: O=Cc1cccc2cc[nH]c12] |
| F | 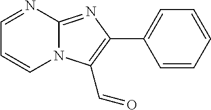 [SMILES: O=Cc1c(-c2ccccc2)nc2ncccn12] | 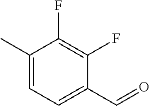 | 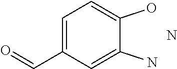 |
| G | 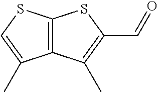 | 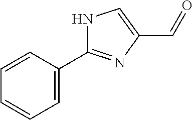 [SMILES: O=Cc1c[nH]c(-c2ccccc2)n1] | 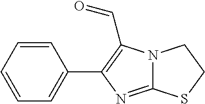 [SMILES: O=Cc1c(-c2ccccc2)nc2n1CCS2] |
| H | 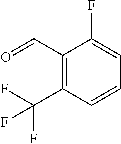 [SMILES: O=Cc1c(F)cccc1C(F)(F)F] | 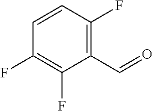 [SMILES: O=Cc1c(F)ccc(F)c1F] | 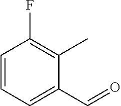 |

Plate 3

| | 04 | 05 | 06 |
|---|---|---|---|
| A | 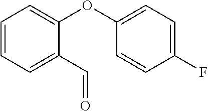 [SMILES: O=Cc1ccccc1Oc1ccc(F)cc1] | 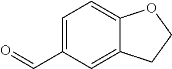 [SMILES: O=Cc1ccc2c(c1)CCO2] | 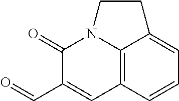 [SMILES: O=Cc1cc2cccc3c2n(c1=O)CC3] |
| B | 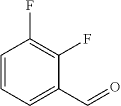 | 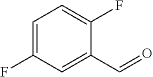 | 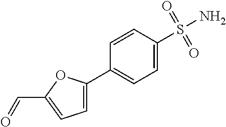 [SMILES: NS(=O)(=O)c1ccc(-c2ccc(C=O)o2)cc1] |
| C | 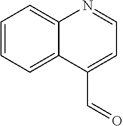 [SMILES: O=Cc1ccnc2ccccc12] | 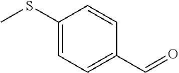 | 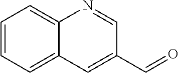 [SMILES: O=Cc1cnc2ccccc2c1] |
| D | 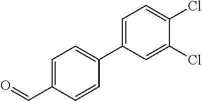 [SMILES: O=Cc1ccc(-c2ccc(Cl)c(Cl)c2)cc1] | 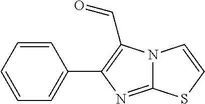 [SMILES: O=Cc1c(-c2ccccc2)nc2sccn12] | 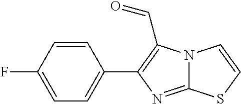 [SMILES: O=Cc1c(-c2ccc(F)cc2)nc2sccn12] |
| E | 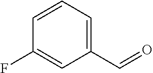 | 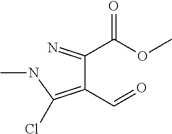 | 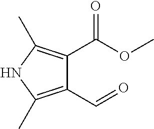 |
| F | 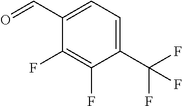 [SMILES: O=Cc1ccc(C(F)(F)F)c(F)c1F] | 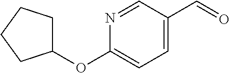 [SMILES: O=Cc1ccc(OC2CCCC2)nc1] | 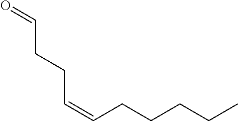 |
| G | 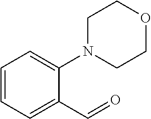 [SMILES: O=Cc1ccccc1N1CCOCC1] | 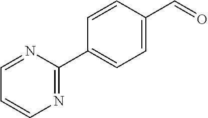 [SMILES: O=Cc1ccc(-c2ncccn2)cc1] | 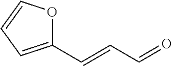 |
| H | 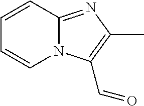 | 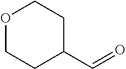 | 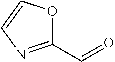 |

Plate 4

| | 04 | 05 | 06 |
|---|---|---|---|
| A | 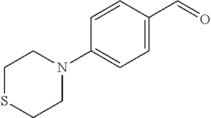 [SMILES: O=Cc1ccc(N2CCSCC2)cc1] | 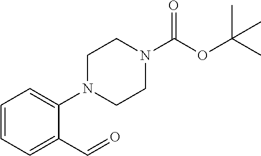 [SMILES: CC(C)(C)OC(=O)N1CCN(c2ccccc2C=O)CC1] | 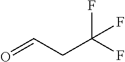 |
| B | 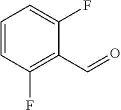 | 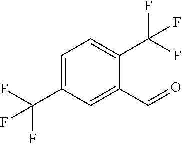 [SMILES: O=Cc1cc(C(F)(F)F)ccc1C(F)(F)F] | 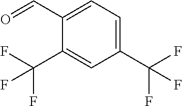 [SMILES: O=Cc1ccc(C(F)(F)F)cc1C(F)(F)F] |
| C | 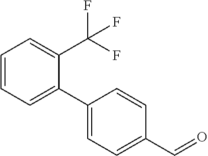 [SMILES: O=Cc1ccc(-c2ccccc2C(F)(F)F)cc1] | | 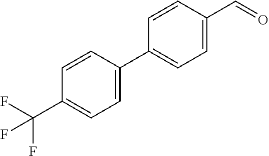 [SMILES: O=Cc1ccc(-c2ccc(C(F)(F)F)cc2)cc1] |
| D | 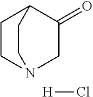 | 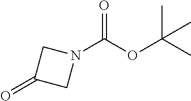 | 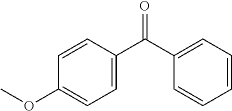 [SMILES: COc1ccc(C(=O)c2ccccc2)cc1] |
| E | 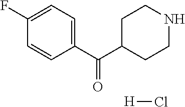 | 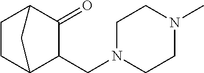 [SMILES: CN1CCN(CC2C(=O)C3CCC2C3)CC1] | 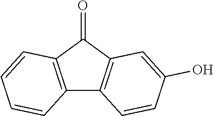 [SMILES: O=C1c2ccccc2-c2ccc(O)cc21] |
| F | 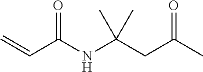 | 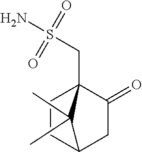 | 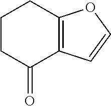 |
| G | 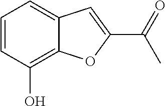 | 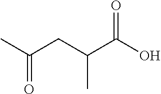 | 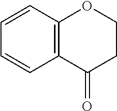 [SMILES: O=C1CCOc2ccccc21] |

Plate 1

| | 07 | 08 | 09 |
|---|---|---|---|
| A | 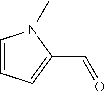 | 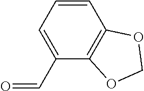 [SMILES: O=Cc1cccc2c1OCO2] | 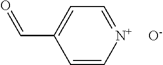 |
| B | 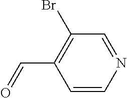 | 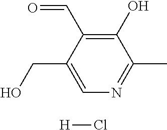 | 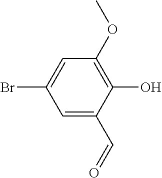 |
| C | 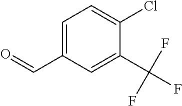 [SMILES: O=Cc1ccc(Cl)c(C(F)(F)F)c1] | 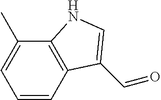 | 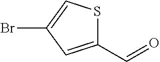 |
| D | 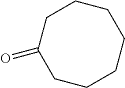 | 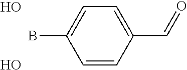 | |
| E | 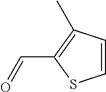 | 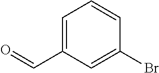 | 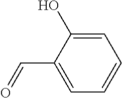 |
| F | 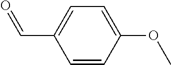 | 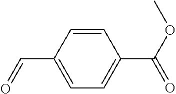 | 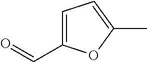 |
| G | | 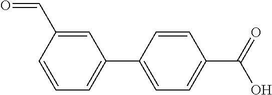 [SMILES: O=Cc1cccc(-c2ccc(C(=O)O)cc2)c1] | 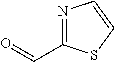 |
| H | 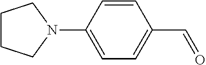 [SMILES: O=Cc1ccc(N2CCCC2)cc1] | 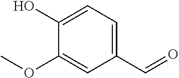 | 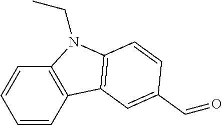 [SMILES: CCn1c2ccccc2c2cc(C=O)ccc21] |

Plate 2

| | 07 | 08 | 09 |
|---|---|---|---|
| A | 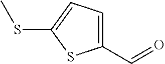 | 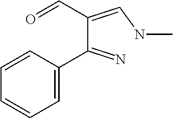 | 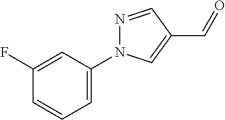 [SMILES: O=Cc1cnn(-c2cccc(F)c2)c1] |
| B | 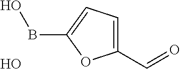 | 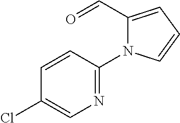 [SMILES: O=Cc1cccn1-c1ccc(Cl)cn1] | 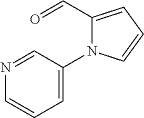 [SMILES: O=Cc1cccn1-c1cccnc1] |
| C | 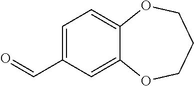 [SMILES: O=Cc1ccc2c(c1)OCCCO2] | 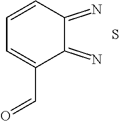 | 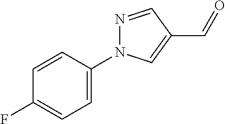 [SMILES: O=Cc1cnn(-c2ccc(F)cc2)c1] |
| D | 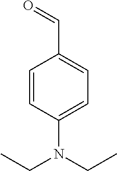 | 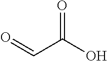 | |
| E | 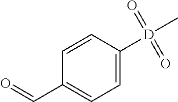 | 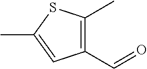 | 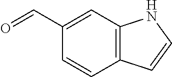 [SMILES: O=Cc1ccc2cc[nH]c2c1] |
| F | 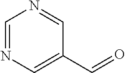 | 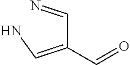 | 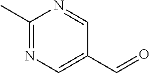 |
| G | | 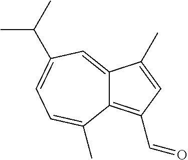 [SMILES: Cc1cc(C=O)c2c(C)ccc(C(C)C)cc1-2] | 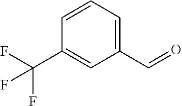 [SMILES: O=Cc1cccc(C(F)(F)F)c1] |
| H | 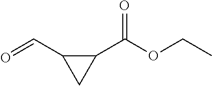 | 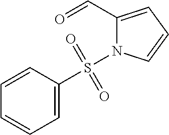 [SMILES: O=Cc1cccn1S(=O)(=O)c1ccccc1] | 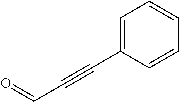 |

Plate 3

| | 07 | 08 | 09 |
|---|---|---|---|
| A | 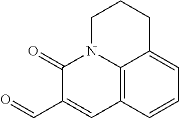 [SMILES: O=Cc1cc2cccc3c2n(c1=O)CCC3] | 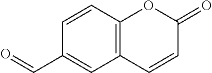 [SMILES: O=Cc1ccc2oc(=O)ccc2c1] | 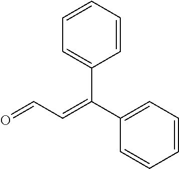 [SMILES: O=CC=C(c1ccccc1)c1ccccc1] |
| B | 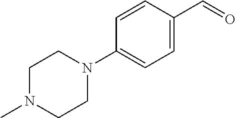 | 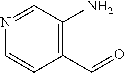 | 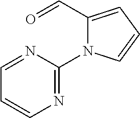 [SMILES: O=Cc1cccn1-c1ncccn1] |
| C | 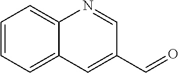 [SMILES: O=Cc1cnc2ccccc2c1] | 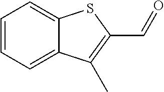 | 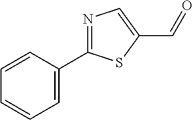 [SMILES: O=Cc1cnc(-c2ccccc2)s1] |
| D | 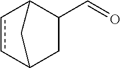 | 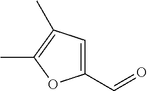 | |
| E | 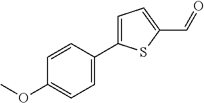 | 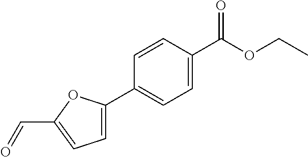 [SMILES: CCOC(=O)c1ccc(-c2ccc(C=O)o2)cc1] | 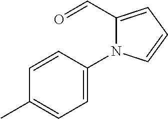 |
| F | 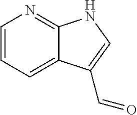 [SMILES: O=Cc1c[nH]c2ncccc12] | 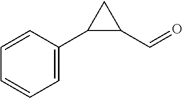 [SMILES: O=CC1CC1c1ccccc1] | 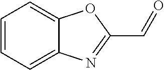 [SMILES: O=Cc1nc2ccccc2o1] |
| G | | 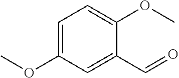 | 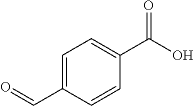 [SMILES: O=Cc1ccc(C(=O)O)cc1] |
| H | 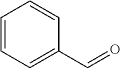 | 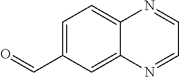 [SMILES: O=Cc1ccc2nccnc2c1] | 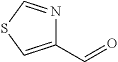 |

Plate 4

| | 07 | 08 | 09 |
|---|---|---|---|
| A | 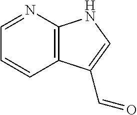 [SMILES: O=Cc1c[nH]c2ncccc12] | 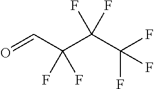 [SMILES: O=CC(F)(F)C(F)(F)C(F)(F)F] | 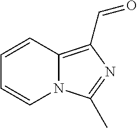 |
| B | 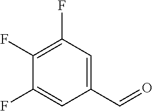 [SMILES: O=Cc1cc(F)c(F)c(F)c1] | 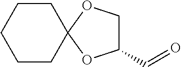 [SMILES: O=C[C@H]1COC2(CCCCC2)O1] | 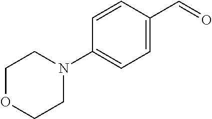 [SMILES: O=Cc1ccc(N2CCOCC2)cc1] |
| C | 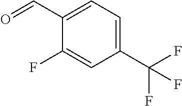 [SMILES: O=Cc1ccc(C(F)(F)F)cc1F] | 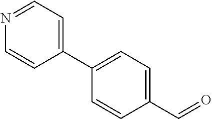 [SMILES: O=Cc1ccc(-c2ccncc2)cc1] | 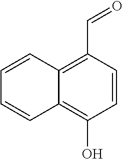 [SMILES: O=Cc1ccc(O)c2ccccc12] |
| D | 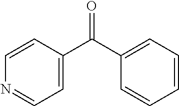 [SMILES: O=C(c1ccccc1)c1ccncc1] | 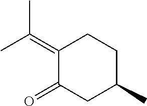 | |
| E | 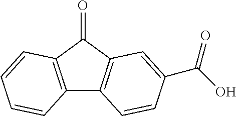 [SMILES: O=C(O)c1ccc2c(c1)C(=O)c1ccccc1-2] | 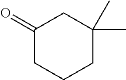 | 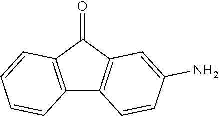 [SMILES: Nc1ccc2c(c1)C(=O)c1ccccc1-2] |
| F | 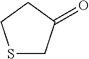 | 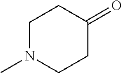 | 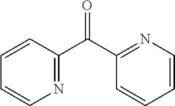 [SMILES: O=C(c1ccccn1)c1ccccn1] |
| G | | 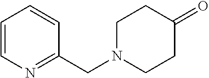 [SMILES: O=C1CCN(Cc2ccccn2)CC1] | 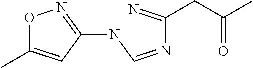 |

Plate 1

| | 10 | 11 | 12 |
|---|---|---|---|
| A | 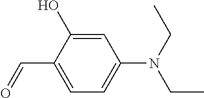 | 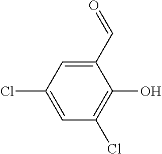 [SMILES: O=Cc1cc(Cl)cc(Cl)c1O] | 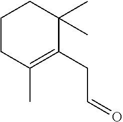 |
| B | 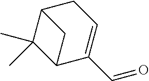 | 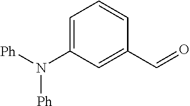 [SMILES: O=Cc1cccc(N(c2ccccc2)c2ccccc2)c1] | 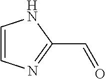 |
| C | 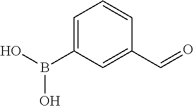 [SMILES: O=Cc1cccc(B(O)O)c1] | 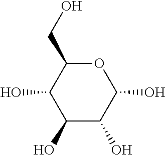 [SMILES: OC[C@H]1O[C@H](O)[C@H](O)[C@@H](O)[C@@H]1O] | 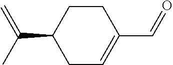 |
| D | 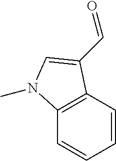 | 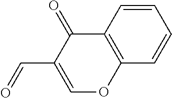 [SMILES: O=Cc1coc2ccccc2c1=O] | 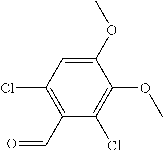 |
| E | 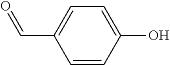 | 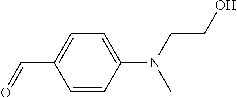 | 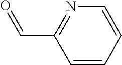 |
| F | 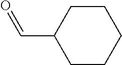 | | 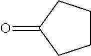 |
| G | 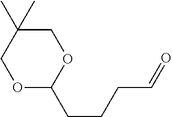 | 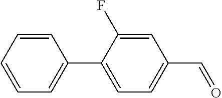 [SMILES: O=Cc1ccc(-c2ccccc2)c(F)c1] | 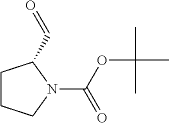 |
| H | 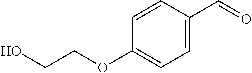 [SMILES: O=Cc1ccc(OCCO)cc1] | 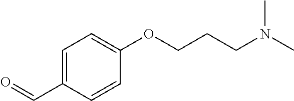 | 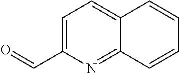 [SMILES: O=Cc1ccc2ccccc2n1] |

Plate 2

| | 10 | 11 | 12 |
|---|---|---|---|
| A | 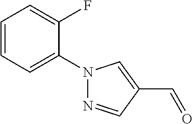 [SMILES: O=Cc1cnn(-c2ccccc2F)c1] | 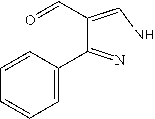 [SMILES: N=C(/C(C=O)=C\N)c1ccccc1] | 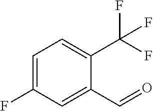 [SMILES: O=Cc1cc(F)ccc1C(F)(F)F] |
| B | 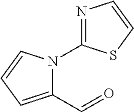 [SMILES: O=Cc1cccn1-c1nccs1] | 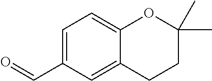 | 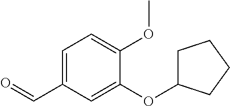 [SMILES: COc1ccc(C=O)cc1OC1CCCC1] |
| C | 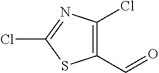 | 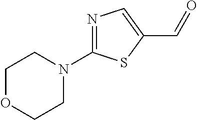 [SMILES: O=Cc1cnc(N2CCOCC2)s1] | 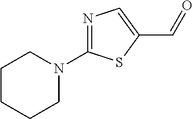 [SMILES: O=Cc1cnc(N2CCCCC2)s1] |
| D | 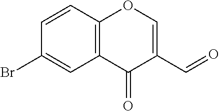 [SMILES: O=Cc1coc2ccc(Br)cc2c1=O] | 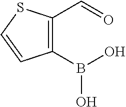 | 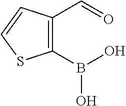 |
| E | 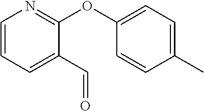 [SMILES: Cc1ccc(Oc2ncccc2C=O)cc1] | 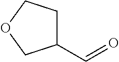 | 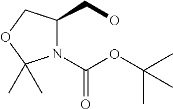 |
| F | 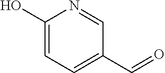 | | 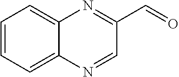 [SMILES: O=Cc1cnc2ccccc2n1] |
| G | 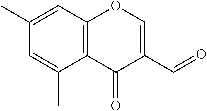 | 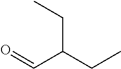 | 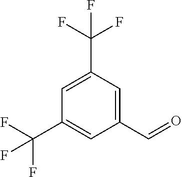 [SMILES: O=Cc1cc(C(F)(F)F)cc(C(F)(F)F)c1] |
| H | 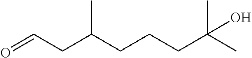 | 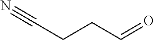 | 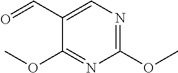 |

Plate 3

| | 10 | 11 | 12 |
|---|---|---|---|
| A | 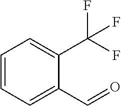 [SMILES: O=Cc1ccccc1C(F)(F)F] | 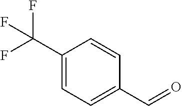 [SMILES: O=Cc1ccc(C(F)(F)F)cc1] | 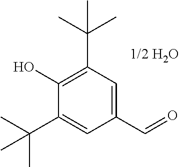 [SMILES: CC(C)(C)c1cc(C=O)cc(C(C)(C)C)c1O] |
| B | 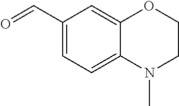 | 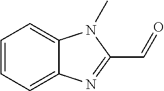 | 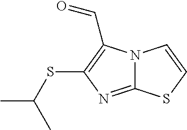 |
| C | 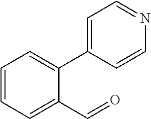 [SMILES: O=Cc1ccccc1-c1ccncc1] | 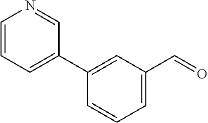 [SMILES: O=Cc1cccc(-c2cccnc2)c1] | 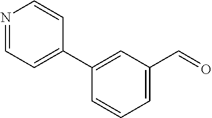 [SMILES: O=Cc1cccc(-c2ccncc2)c1] |
| D | 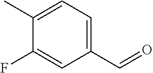 | 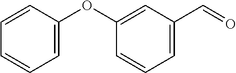 [SMILES: O=Cc1cccc(Oc2ccccc2)c1] | 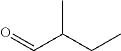 |
| E | 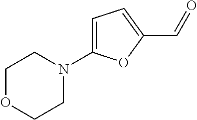 [SMILES: O=Cc1ccc(N2CCOCC2)o1] | 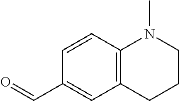 | 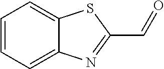 [SMILES: O=Cc1nc2ccccc2s1] |
| F | 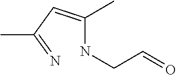 | | 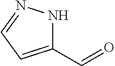 |
| G | 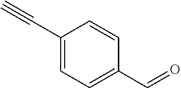 | 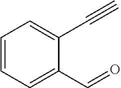 | 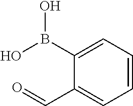 [SMILES: O=Cc1ccccc1B(O)O] |
| H | 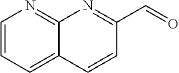 [SMILES: O=Cc1ccc2cccnc2n1] | 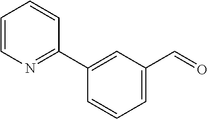 [SMILES: O=Cc1cccc(-c2ccccn2)c1] | 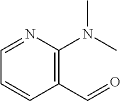 |

Plate 4

| | 10 | 11 | 12 |
|---|---|---|---|
| A | 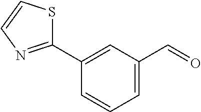 [SMILES: O=Cc1cccc(-c2nccs2)c1] | 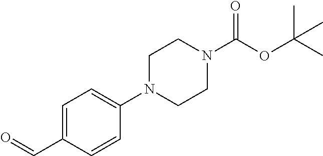 [SMILES: CC(C)(C)OC(=O)N1CCN(c2ccc(C=O)cc2)CC1] | 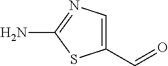 |
| B | 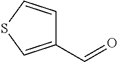 | 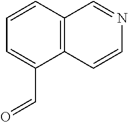 [SMILES: O=Cc1cccc2cnccc12] | 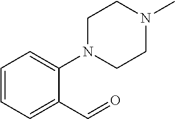 |
| C | 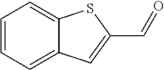 [SMILES: O=Cc1cc2ccccc2s1] | 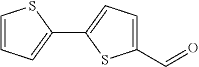 [SMILES: O=Cc1ccc(-c2cccs2)s1] | 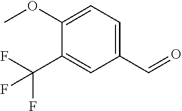 |
| D | 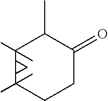 | 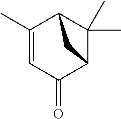 | 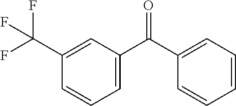 [SMILES: O=C(c1ccccc1)c1cccc(C(F)(F)F)c1] |
| E | 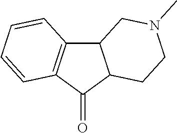 | 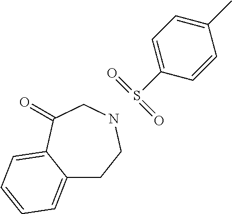 | 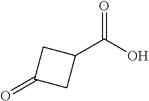 |
| F | 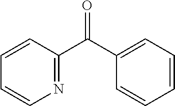 [SMILES: O=C(c1ccccc1)c1ccccn1] | | 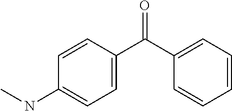 [SMILES: CNc1ccc(C(=O)c2ccccc2)cc1] |
| G | 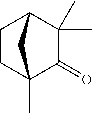 | 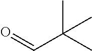 | |

In one embodiment, the compound is a compound is represented by the formula:

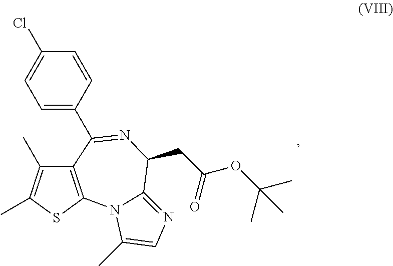 [SMILES: Cc1sc2c(c1C)C(c1ccc(Cl)cc1)=N[C@@H](CC(=O)OC(C)(C)C)c1ncc(C)n1-2]

or a salt, solvate or hydrate thereof.

In certain embodiments, the compound is (racemic) JQ1; in certain embodiments, the compound is (+)-JQ1. In certain embodiments, the compound is a compound selected from the group consisting of:

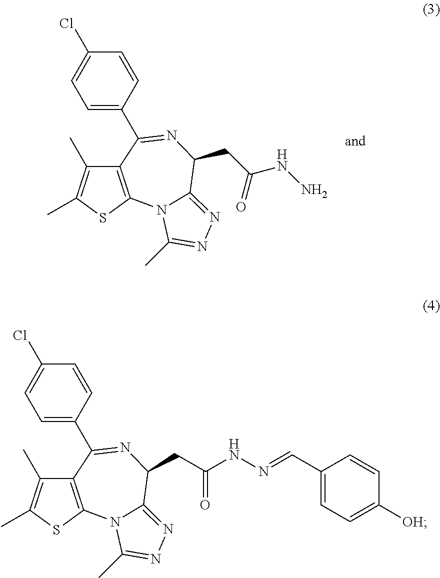 [SMILES: Cc1sc2c(c1C)C(c1ccc(Cl)cc1)=N[C@@H](CC(=O)NN)c1nnc(C)n1-2 ; Cc1sc2c(c1C)C(c1ccc(Cl)cc1)=N[C@@H](CC(=O)N/N=C/c1ccc(O)cc1)c1nnc(C)n1-2]

or a salt, solvate, or hydrate thereof.

Additional examples of compounds include compounds according to any of the follow formulae:

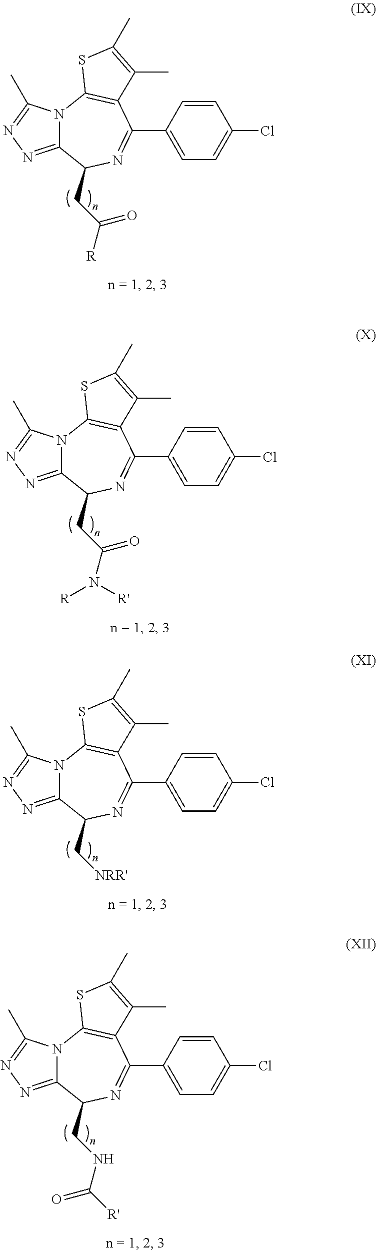

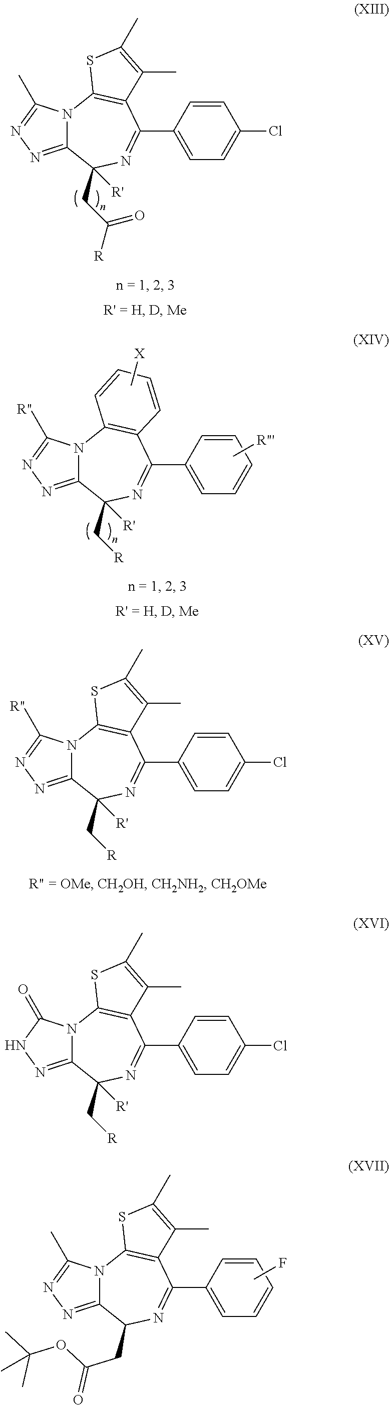

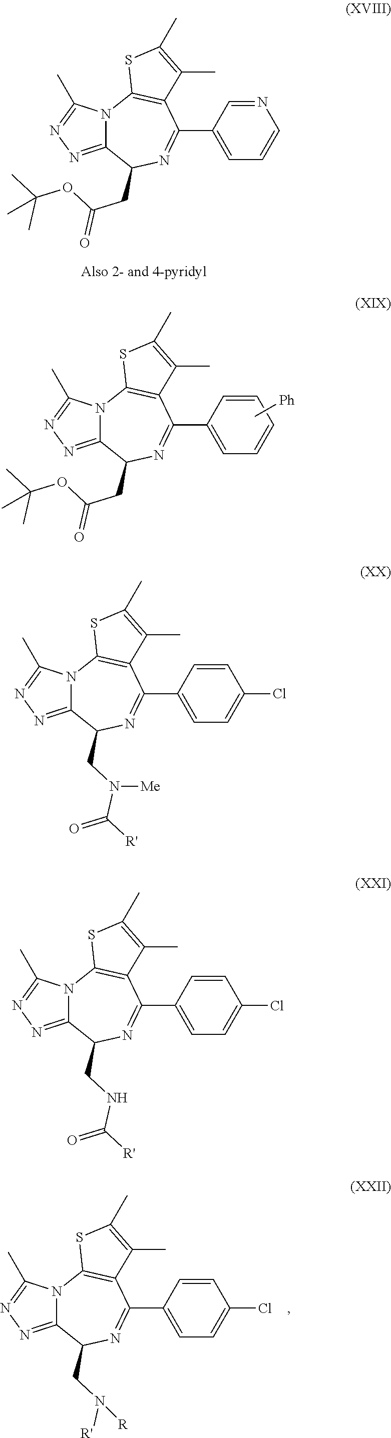

or a salt, solvate, or hydrate thereof.

In Formulae IX-XXII, R and R′ can be, e.g., H, aryl, substituted aryl, heteroaryl, heteroaryl, heterocycloalkyl, —C₁-C₈ alkyl, —C₂-C₈ alkenyl, —C₂-C₈ alkynyl, —C₃-C₁₂ cycloalkyl, substituted —C₃-C₁₂ cycloalkyl, —C₃-C₁₂ cycloalkenyl, or substituted —C₃-C₁₂ cycloalkenyl, each of which may be optionally substituted. In Formulae XIV, X can be any substituent for an aryl group as described herein.

Compounds of the invention can be prepared by a variety of methods, some of which are known in the art. For instance, the chemical Examples provided hereinbelow provide synthetic schemes for the preparation of the compound JQ1 (as the racemate) and the enantiomers (+)-JQ1 and (−)-JQ1 (see Schemes S1 and S2). A variety of compounds of Formulae (I)-(VIII) can be prepared by analogous methods with substitution of appropriate starting materials.

For example, starting from JQ1, the analogous amine can be prepared as shown in Scheme 1, below.

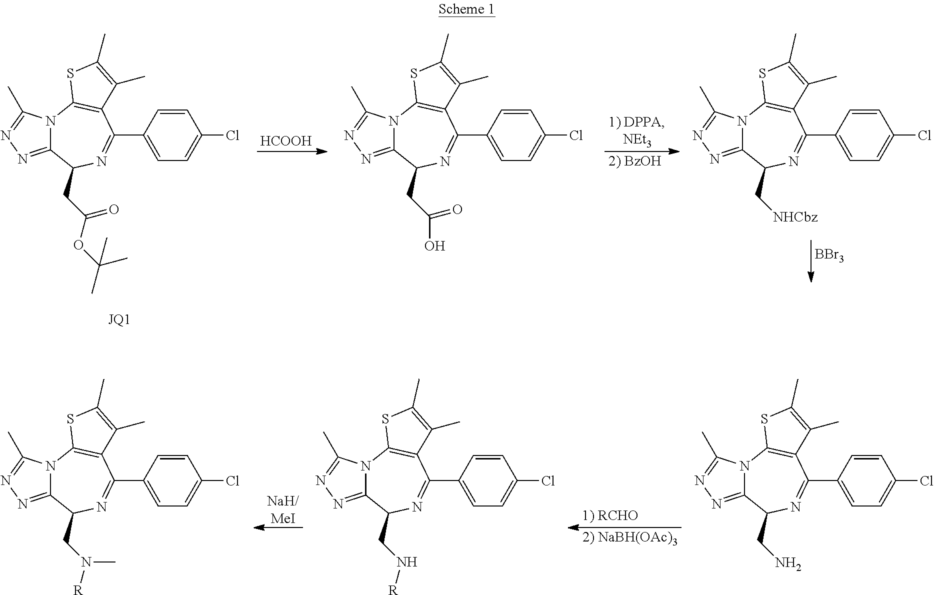

As shown in Scheme 1, hydrolysis of the t-butyl ester of JQ1 affords the carboxylic acid, which is treated with diphenylphosphoryl azide (DPPA) and subjected to Curtius rearrangement conditions to provide the Cbz-protected amine, which is then deprotected to yield the amine. Subsequent elaboration of the amine group, e.g., by reductive amination yields secondary amines, which can be further alkylated to provide tertiary amines.

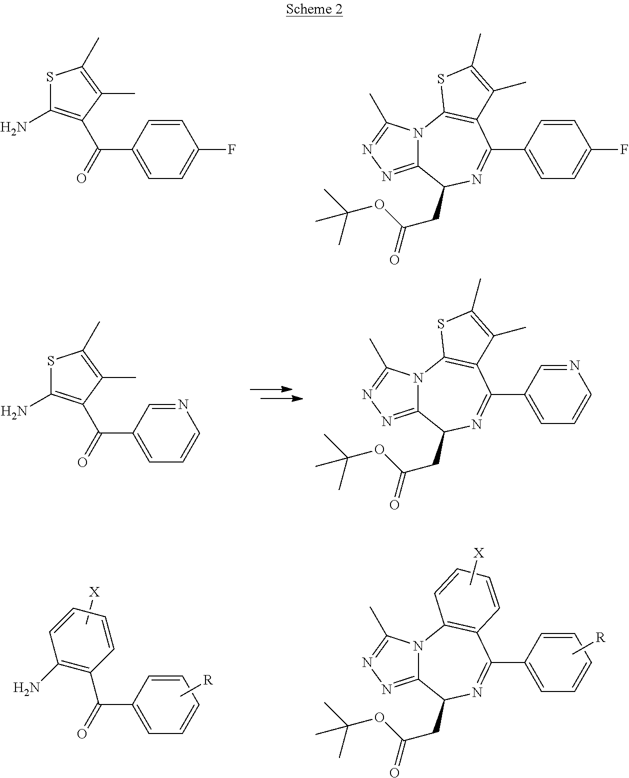

Scheme 2 shows the synthesis of further examples of the compounds of the invention, e.g., of Formula I, in which the fused ring core is modified (e.g., by substitution of a different aromatic ring as Ring A in Formula I). Use of aminodiarylketones having appropriate functionality (e.g., in place of the aminodiarylketone S2 in Scheme S1, infra) provides new compounds having a variety of fused ring cores and/or aryl group appendages (corresponding to group R in Formula I). Such aminodiarylketones are commercially available or can be prepared by a variety of methods, some of which are known in the art.

Scheme 3 provides additional exemplary synthetic schemes for preparing further compounds of the invention.

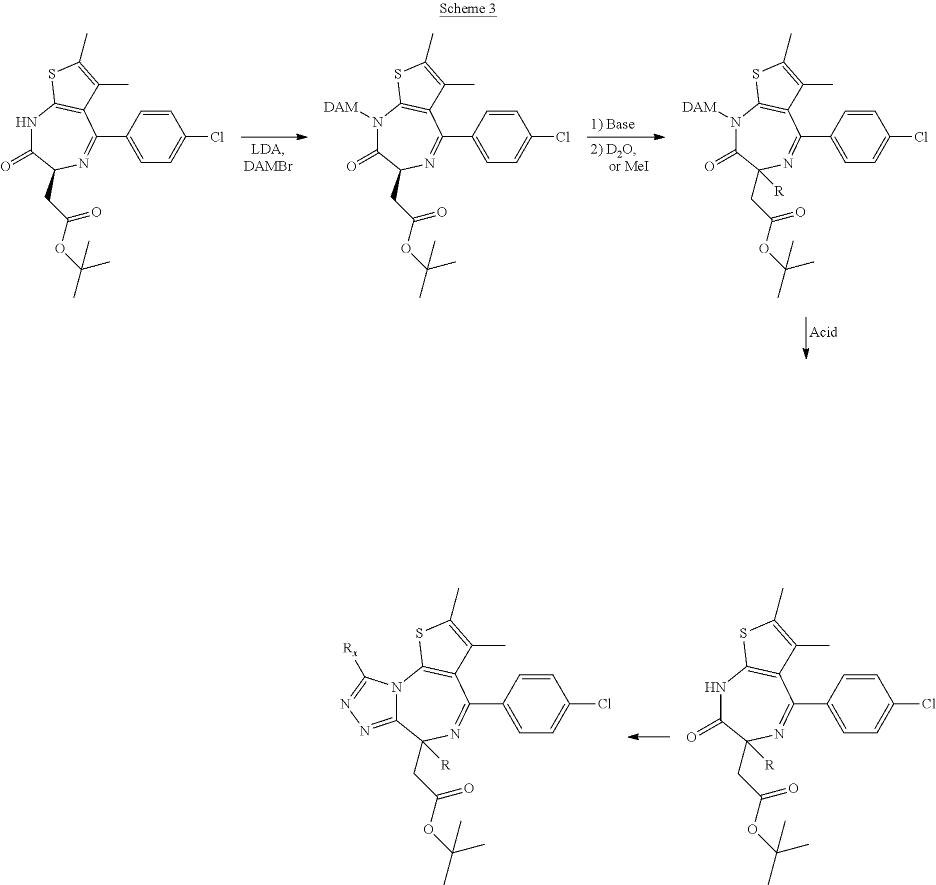

As shown in Scheme 3, a fused bicyclic precursor (see Scheme S1, infra, for synthesis of this compound) is functionalized with a moiety R (DAM=dimethylaminomethylene protecting group) and then elaborated by reaction with a hydrazide to form the tricyclic fused core. Substituent Rₓ can be varied by selection of a suitable hydrazide.

Additional examples of compounds of the invention (which can be prepared by the methods described herein) include:

Amides:

Amides can be prepared, e.g., by preparation of a corresponding carboxylic acid or ester, followed by amidation with an appropriate amine using standard conditions. In certain embodiments, an amide provides a two-carbon “linker” with a terminal terminal nitrogen-containing ring (e.g., pyridyl, piperidyl, piperazinyl, imidazolyl (including N-methyl-imidazolyl), morpholinyl, and the like. Exemplary amide structures include:

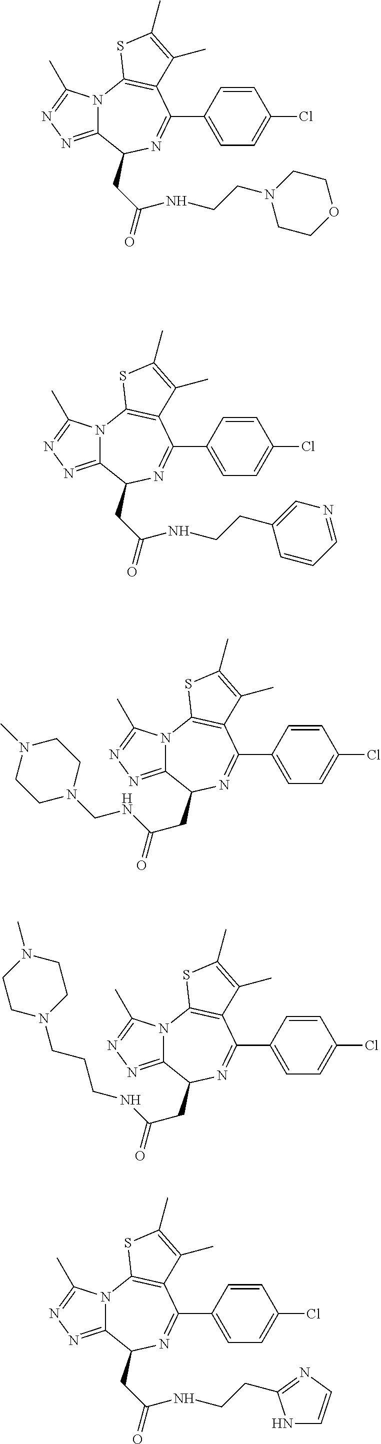 [SMILES: Cc1sc2c(c1C)C(c1ccc(Cl)cc1)=N[C@@H](CC(=O)NCCN1CCOCC1)c1nnc(C)n1-2 ; Cc1sc2c(c1C)C(c1ccc(Cl)cc1)=N[C@@H](CC(=O)NCCc1cccnc1)c1nnc(C)n1-2 ; Cc1sc2c(c1C)C(c1ccc(Cl)cc1)=N[C@@H](CC(=O)NCN1CCN(C)CC1)c1nnc(C)n1-2 ; Cc1sc2c(c1C)C(c1ccc(Cl)cc1)=N[C@@H](CC(=O)NCCCN1CCN(C)CC1)c1nnc(C)n1-2 ; Cc1sc2c(c1C)C(c1ccc(Cl)cc1)=N[C@@H](CC(=O)NCCc1ncc[nH]1)c1nnc(C)n1-2]

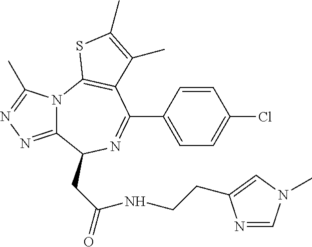 [SMILES: Cc1sc2c(c1C)C(c1ccc(Cl)cc1)=N[C@@H](CC(=O)NCCc1cn(C)cn1)c1nnc(C)n1-2]

The use of a two-carbon linker between the amide moiety and the terminal nitrogen-containing ring is preferred.

“Reverse amides”:

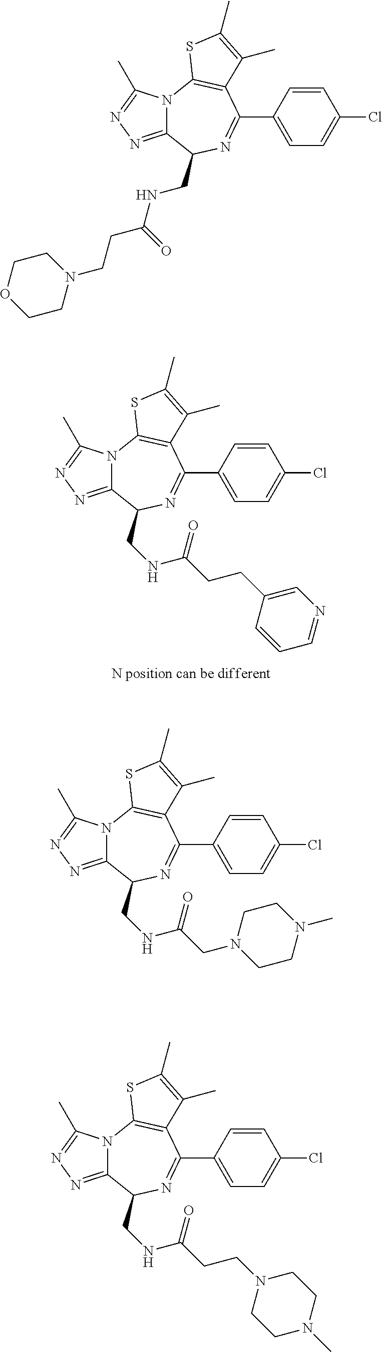 [SMILES: Cc1sc2c(c1C)C(c1ccc(Cl)cc1)=N[C@@H](CNC(=O)CCN1CCOCC1)c1nnc(C)n1-2 ; Cc1sc2c(c1C)C(c1ccc(Cl)cc1)=N[C@@H](CNC(=O)CCc1cccnc1)c1nnc(C)n1-2 ; Cc1sc2c(c1C)C(c1ccc(Cl)cc1)=N[C@@H](CNC(=O)CN1CCN(C)CC1)c1nnc(C)n1-2 ; Cc1sc2c(c1C)C(c1ccc(Cl)cc1)=N[C@@H](CNC(=O)CCN1CCN(C)CC1)c1nnc(C)n1-2]

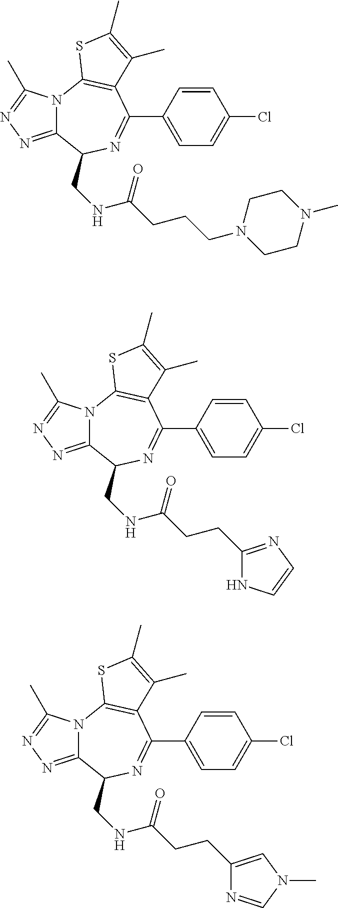 [SMILES: Cc1sc2c(c1C)C(c1ccc(Cl)cc1)=N[C@@H](CNC(=O)CCCN1CCN(C)CC1)c1nnc(C)n1-2 ; Cc1sc2c(c1C)C(c1ccc(Cl)cc1)=N[C@@H](CNC(=O)CCc1ncc[nH]1)c1nnc(C)n1-2 ; Cc1sc2c(c1C)C(c1ccc(Cl)cc1)=N[C@@H](CNC(=O)CCc1cn(C)cn1)c1nnc(C)n1-2]

Secondary Amines:

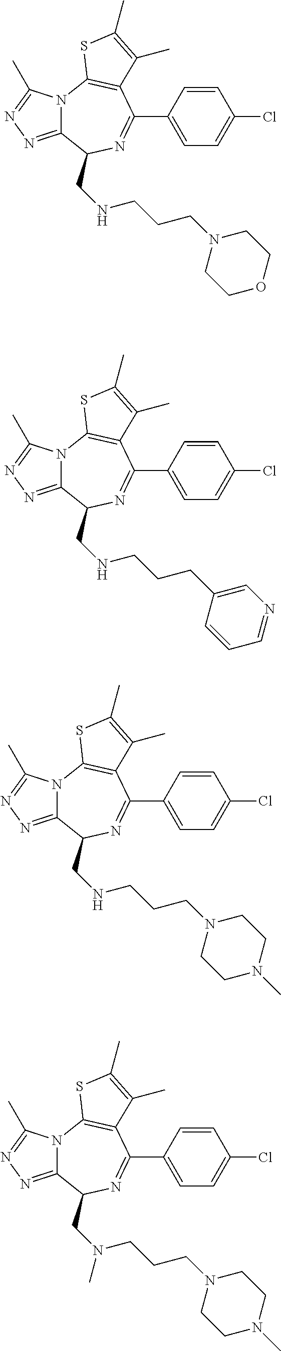 [SMILES: Cc1sc2c(c1C)C(c1ccc(Cl)cc1)=N[C@@H](CNCCCN1CCOCC1)c1nnc(C)n1-2 ; Cc1sc2c(c1C)C(c1ccc(Cl)cc1)=N[C@@H](CNCCCc1cccnc1)c1nnc(C)n1-2 ; Cc1sc2c(c1C)C(c1ccc(Cl)cc1)=N[C@@H](CNCCCN1CCN(C)CC1)c1nnc(C)n1-2 ; Cc1sc2c(c1C)C(c1ccc(Cl)cc1)=N[C@@H](CN(C)CCCN1CCN(C)CC1)c1nnc(C)n1-2]

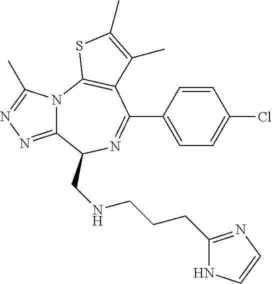 [SMILES: Cc1sc2c(c1C)C(c1ccc(Cl)cc1)=N[C@@H](CNCCCc1ncc[nH]1)c1nnc(C)n1-2]

Boronic Acids:

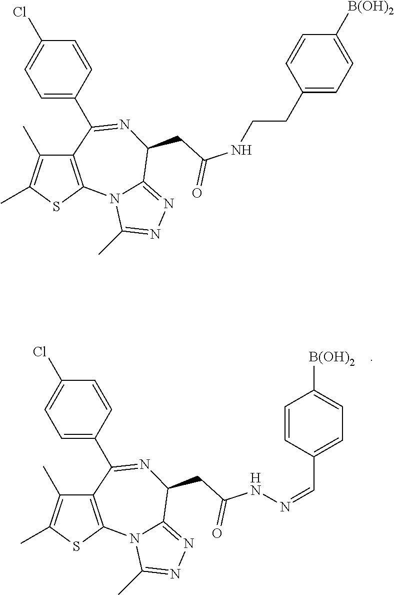 [SMILES: Cc1sc2c(c1C)C(c1ccc(Cl)cc1)=N[C@@H](CC(=O)NCCc1ccc(B(O)O)cc1)c1nnc(C)n1-2 ; Cc1sc2c(c1C)C(c1ccc(Cl)cc1)=N[C@@H](CC(=O)N/N=C\c1ccc(B(O)O)cc1)c1nnc(C)n1-2]

In certain embodiments, a compound having at least one chiral center is present in racemic form. In certain embodiments, a compound having at least one chiral center is enantiomerically enriched, i.e., has an enantiomeric excess (e.e.) of at least about 10%, 20%, 30%, 40%, 50%, 60%, 70%, 80%, 85%, 90%, 90%, 95%, 99%, 99% or 100%. In certain embodiments, a compound has the same absolute configuration as the compound (+)-JQ1 ((S)-tert-Butyl 2-(4-(4-chlorophenyl)-2,3,9-trimethyl-6H-thieno[3,2-f][1,2,4]triazolo[4,3-a][1,4]diazepin-6-yl)acetate) described herein.

In certain embodiments of any of the Formulae disclosed herein, the compound is not represented by the following structure:

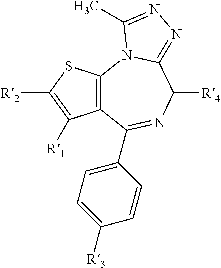

in which:

R′₁ is C₁-C₄ alkyl;

R′₂ is hydrogen, halogen, or C₁-C₄ alkyl optionally substituted with a halogen atom or a hydroxyl group;

R′₃ is a halogen atom, phenyl optionally substituted by a halogen atom, C₁-C₄ alkyl, C₁-C₄ alkoxyy, or cyano; —NR₅—(CH₂)ₘ—R₆ wherein R₅ is a hydrogen atom or C₁-C₄ alkyl, m is an integer of 0-4, and R₆ is phenyl or pyridyl optionally substituted by a halogen atom; or —NR₇—CO—(CH₂)ₙ—R₈ wherein R₇ is a hydrogen atom or C₁-C₄ alkyl, n is an integer of 0-2, and R₈ is phenyl or pyridyl optionally substituted by a halogen atom; and

R′₄ is —(CH₂)ₐ—CO—NH—R₉ wherein a is an integer of 1-4, and R₉ is C₁-C₄ alkyl; C₁-C₄ hydroxyalkyl; C₁-C₄ alkoxy; or phenyl or pyridyl optionally substituted by C₁-C₄ alkyl, C₁-C₄ alkoxy, amino or a hydroxyl group or —(CH₂)_{b}—COOR₁₀ wherein b is an integer of 1-4, and R₁₀ is C₁-C₄ alkyl.

The term “pharmaceutically acceptable salt” also refers to a salt prepared from a compound disclosed herein (e.g., JQ1, a compound of Formulas I-XXII) or any other compound delineated herein, having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like. The term “pharmaceutically acceptable salt” also refers to a salt prepared from a compound disclosed herein, or any other compound delineated herein, having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

### Inhibitory Nucleic Acids

Inhibitory nucleic acid molecules of the invention are those oligonucleotides that inhibit the expression of a BET protein or nucleic acid molecule (e.g., Brd2, Brd3, Brd4). Such oligonucleotides include single and double stranded nucleic acid molecules (e.g., DNA, RNA, and analogs thereof) that bind a nucleic acid molecule that encodes a BET polypeptide (e.g., antisense molecules, siRNA, shRNA) as well as nucleic acid molecules that bind directly to a BET polypeptide (e.g., Brd2, Brd3, Brd4) to modulate its biological activity (e.g., aptamers).

Ribozymes

Catalytic RNA molecules or ribozymes that include an antisense BET sequence of the present invention can be used to inhibit expression of a BET nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an sirNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of an BET gene can be used to design small interfering RNAs (siRNAs). The 21 to 25 nucleotide siRNAs may be used, for example, as therapeutics to treat a vascular disease or disorder.

The inhibitory nucleic acid molecules of the present invention may be employed as double-stranded RNAs for RNA interference (RNAi)-mediated knock-down of BET expression. In one embodiment, BET expression is reduced in an adipocyte or pre-adipocyte. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Screening Methods

As described above, the invention provides specific examples of chemical compounds, including JQ1, as well as other substituted compounds that bind a bromodomain binding pocket and that inhibit adipogenesis, adipocyte differentiation, and adipocyte biological activity (e.g., fat synthesis, fat accumulation. However, the invention is not so limited. The invention further provides a simple means for identifying agents (including nucleic acids, peptides, small molecule inhibitors, and mimetics) that are capable of inhibiting adipogenesis, adipocyte differentiation, and adipocyte biological activity (e.g., fat synthesis, fat accumulation. Such compounds are also expected to be useful for the treatment or prevention of a metabolic syndrome, obesity, type II diabetes, insulin resistance, and related disorders characterized by undesirable alterations in metabolism or fat accumulation.

In particular, certain aspects of the invention are based at least in part on the discovery that agents that reduce the biological activity of a BET family member polypeptide are likely useful as therapeutics for the treatment or prevention of metabolic syndrome, obesity, type II diabetes, insulin resistance, and related disorders characterized by undesirable alterations in metabolism or fat accumulation. In particular embodiments, the effect of a compound or other agent of the invention is analyzed by assaying adipogenesis, adipocyte differentiation, adipocyte biological activity (e.g., fat synthesis, fat accumulation), the expression of transcription factors and other proteins that function in adipogenesis, weight gain, and fat accumulation (e.g., visceral fat, subcutaneous fat, fatty liver). Agents and compounds of the invention that reduce adipogenesis, adipocyte differentiation, adipocyte biological activity (e.g., fat synthesis, fat accumulation), the expression of transcription factors and other proteins that function in adipogenesis, weight gain, and fat accumulation (e.g., visceral fat, subcutaneous fat, fatty liver) are identified as useful for the treatment or prevention of metabolic syndrome, obesity, and related disorders characterized by undesirable alterations in metabolism.

Virtually any agent that specifically binds to a BET family member or that reduces the biological activity of a BET family member may be employed in the methods of the invention. Methods of the invention are useful for the high-throughput low-cost screening of candidate agents that reduce, slow, or otherwise inhibit adipogenesis, adipocyte differentiation, adipocyte biological activity (e.g., fat synthesis, fat accumulation), the expression of transcription factors and other proteins that function in adipogenesis, weight gain, and fat accumulation (e.g., visceral fat, subcutaneous fat, fatty liver) for the treatment or prevention of metabolic syndrome, obesity, and related disorders characterized by undesirable alterations in metabolism. A candidate agent that specifically binds to a bromodomain of a BET family member is then isolated and tested for activity in an in vitro assay or in vivo assay for its ability to treat metabolic syndrome, obesity, type II diabetes, insulin resistance, and related disorders characterized by undesirable alterations in metabolism or fat accumulation. One skilled in the art appreciates that the effects of a candidate agent on a cell is typically compared to a corresponding control cell not contacted with the candidate agent. Thus, the screening methods include comparing the biological activity of a adipocyte contacted by a candidate agent to the biological activity of an untreated control adipocyte. In other embodiments, the biological activity of a candidate agent is assessed using an ob/ob mouse, a db/db mouse, or in another animal model of obesity such as feeding on a high-fat diet.

In other embodiments, the expression or activity of a BET family member in a cell treated with a candidate agent is compared to untreated control samples to identify a candidate compound that decreases the biological activity of a BET family member in the contacted cell. Polypeptide expression or activity can be compared by procedures well known in the art, such as Western blotting, flow cytometry, immunocytochemistry, binding to magnetic and/or a bromodomain-specific antibody-coated beads, in situ hybridization, fluorescence in situ hybridization (FISH), ELISA, microarray analysis, RT-PCR, Northern blotting, or colorimetric assays, such as the Bradford Assay and Lowry Assay.

In one working example, one or more candidate agents is added at varying concentrations to the culture medium containing an adipocyte or pre-adipocyte. An agent that reduces the expression of an adipogenic transcription factor or other adipogenic protein (e.g., C/EBP-α, PPARγ, SREBP, fatty acid synthase (FAS), ACC beta, SCD1, DGAT) expressed in the cell is considered useful in the invention; such an agent may be used, for example, as a therapeutic to prevent, delay, ameliorate, stabilize, or treat a metabolic syndrome, obesity, type II diabetes, insulin resistance, and related disorders characterized by undesirable alterations in metabolism or fat accumulation. Once identified, agents of the invention (e.g., agents that specifically bind to and/or antagonize a bromodomain) may be used to treat metabolic syndrome, obesity, type II diabetes, insulin resistance, and related disorders characterized by undesirable alterations in metabolism or fat accumulation. An agent identified according to a method of the invention is locally or systemically delivered to treat metabolic syndrome, obesity, type II diabetes, insulin resistance, and related disorders characterized by undesirable alterations in metabolism or fat accumulation in situ.

Potential bromodomain antagonists include organic molecules, peptides, peptide mimetics, polypeptides, nucleic acid ligands, aptamers, and antibodies that bind to a BET family member bromodomain and reduce its activity. Candidate agents may be tested for their ability to reduce adipocyte differentiation or biological activity.

### Test Compounds and Extracts

In certain embodiments, BET family member antagonists (e.g., agents that specifically bind and reduce the activity of a bromodomain) are identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include those known as therapeutics for the treatment of metabolic syndrome, obesity, type II diabetes, or other disorders characterized by undesirable alterations in metabolism or fat accumulation. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible. When a crude extract is found to have BET family member bromodomain binding activity further fractionation of the positive lead extract is necessary to isolate molecular constituents responsible for the observed effect. Thus, the goal of the extraction, fractionation, and purification process is the careful characterization and identification of a chemical entity within the crude extract that reduces adipogenesis, adipocyte differentiation, or adipocyte biological activity. Methods of fractionation and purification of such heterogenous extracts are known in the art. If desired, compounds shown to be useful as therapeutics are chemically modified according to methods known in the art.

The present invention provides methods of treating metabolic syndrome, obesity, insulin resistance, and related diseases and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a metabolic syndrome, obesity, type II diabetes, insulin resistance, and related disorders characterized by undesirable alterations in metabolism or fat accumulation or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which undesirable alterations in metabolism, fat accumulation, adipogenesis, adipocyte differentiation, or adipocyte biological activity may be implicated.

In one embodiment, the invention provides a method of monitoring treatment progress. The method includes the step of determining a level of diagnostic marker (Marker) (e.g., weight gain, fatty acid synthesis, triglyceride traficking, insulin resistance, or any other target delineated herein modulated by a compound herein, a protein or indicator thereof, etc.) or diagnostic measurement (e.g., screen, assay) in a subject suffering from or susceptible to a disorder or symptoms thereof associated with undesirable changes in adipogenesis, adipocyte differentiation, or adipocyte biological activity, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Pharmaceutical Therapeutics

In other embodiments, agents discovered to have medicinal value (e.g., JQ1 or a compound of a formula delineated herein) using the methods described herein are useful as a drug or as information for structural modification of existing compounds, e.g., by rational drug design. Such methods are useful for screening agents having an effect on a metabolic syndrome, obesity, type II diabetes, insulin resistance, and related disorders characterized by undesirable alterations in metabolism or fat accumulation.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the metabolic syndrome, obesity, type II diabetes, insulin resistance, and related disorders characterized by undesirable alterations in metabolism or fat accumulation. Generally, amounts will be in the range of those used for other agents used in the treatment of other diseases associated with metabolic syndrome, obesity, type II diabetes, insulin resistance, and related disorders characterized by undesirable alterations in metabolism or fat accumulation, although in certain instances lower amounts will be needed because of the increased specificity of the compound. A compound is administered at a dosage that reduces adipogenesis, adipocyte differentiation, adipocyte biological activity as determined by a method known to one skilled in the art, or using any that assay that measures weight gain or fat accumulation.

### Formulation of Pharmaceutical Compositions

The administration of a compound for the treatment of a metabolic syndrome, obesity, type II diabetes, insulin resistance, and related disorders characterized by undesirable alterations in metabolism or fat accumulation may be by any suitable means that results in a concentration of the therapeutic that, combined with other components, is effective in ameliorating, reducing, or stabilizing a metabolic syndrome, obesity, type II diabetes, insulin resistance, and related disorders characterized by undesirable alterations in metabolism or fat accumulation. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). In one particular embodiment, an agent of the invention is directly administered to adipocytes or to liver. One means for administering a compound of the invention to the liver is via the portal vein or the hepatic artery. Another means of administering a compound of the invention to a tissue or interest is by attachment to a device or solid support (such as a stent or graft).

Human dosage amounts can initially be determined by extrapolating from the amount of compound used in mice, as a skilled artisan recognizes it is routine in the art to modify the dosage for humans compared to animal models. In one embodiment, an agent of the invention is administered orally or systemically at 50 mg/kg. In certain other embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other embodiments, it is envisaged that doses may be in the range of about 5 mg compound/Kg body to about 100 mg compound/Kg body. In other embodiments the doses may be about 5, 10, 15, 20, 25, 30, 35, 40, 45, 50, 55, 60, 65, 70, 75, 80, 85, 90, 95, 100 mg/Kg body weight. Of course, this dosage amount may be adjusted upward or downward, as is routinely done in such treatment protocols, depending on the results of the initial clinical trials and the needs of a particular patient.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a metabolic syndrome, obesity, type II diabetes, insulin resistance, and related disorders characterized by undesirable alterations in metabolism or fat accumulation by using carriers or chemical derivatives to deliver the therapeutic agent to a particular cell type (e.g., adipocyte) or tissue (visceral fat, ectopic fat, fatty liver). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a metabolic syndrome, obesity, type II diabetes, insulin resistance, and related disorders characterized by undesirable alterations in metabolism or fat accumulation, the composition may include suitable parenterally acceptable carriers and/or excipients. The active therapeutic agent(s) may be incorporated into micro spheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active anti-metabolic syndrome therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutaminine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms For Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active therapeutic substance). The coating may be applied on the solid dosage form in a similar manner as that described in Encyclopedia of Pharmaceutical Technology, supra.

At least two therapeutics may be mixed together in the tablet, or may be partitioned. In one example, the first active therapeutic is contained on the inside of the tablet, and the second active therapeutic is on the outside, such that a substantial portion of the second therapeutic is released prior to the release of the first therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Combination Therapies

Optionally, a therapeutic for the treatment of metabolic syndrome, obesity, type II diabetes, insulin resistance, and related disorders characterized by undesirable alterations in metabolism or fat accumulation is administered in combination with any other standard therapy for treating a metabolic syndrome, insulin resistance, type II diabetes or obesity; such methods are known to the skilled artisan and described in Remington's Pharmaceutical Sciences by E. W. Martin. If desired, agents of the invention (e.g., JQ1, compounds of formulas delineated herein, and derivatives thereof) are administered in combination with any conventional therapeutic useful for the treatment of a metabolic syndrome, obesity, type II diabetes, insulin resistance, and related disorders characterized by undesirable alterations in metabolism or fat accumulation. These agents could include anti-diabetic medications (such as sulfonylureas, oral hypoglycemic agents, PPAR agonists or antagonists), cardiovascular drugs (such as antihypertensives, antianginal medications), and anti-inflammatory drugs (such as corticosteroids, HDAC inhibitors, TNF-alpha modulators).

### Kits or Pharmaceutical Systems

The present compositions may be assembled into kits or pharmaceutical systems for use in ameliorating a metabolic syndrome, obesity, type II diabetes, insulin resistance, and related disorders characterized by undesirable alterations in metabolism or fat accumulation. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### I. Chemical Examples

### Synthesis and Methods of Preparation

Compounds of the invention can be synthesized by methods described herein, and/or according to methods known to one of ordinary skill in the art in view of the description herein.

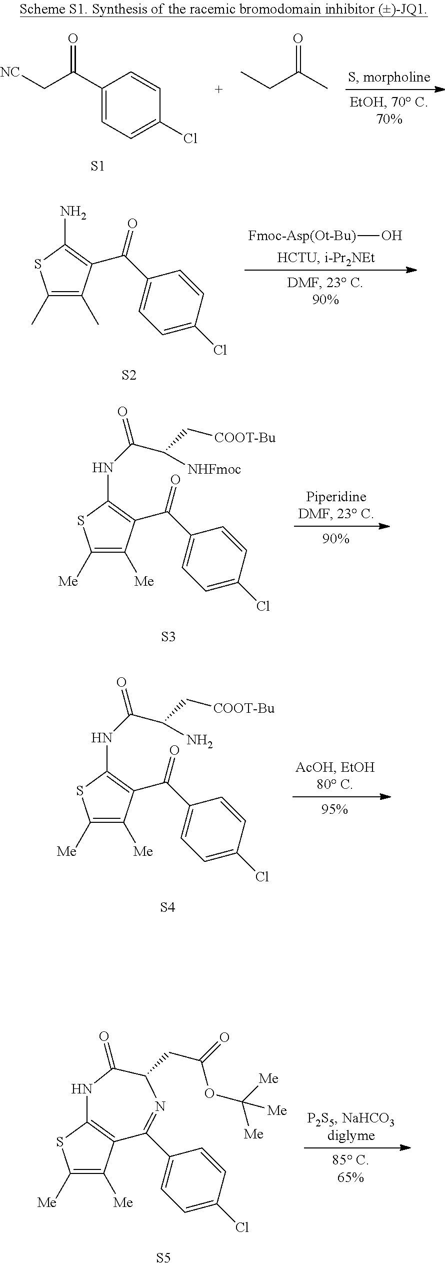

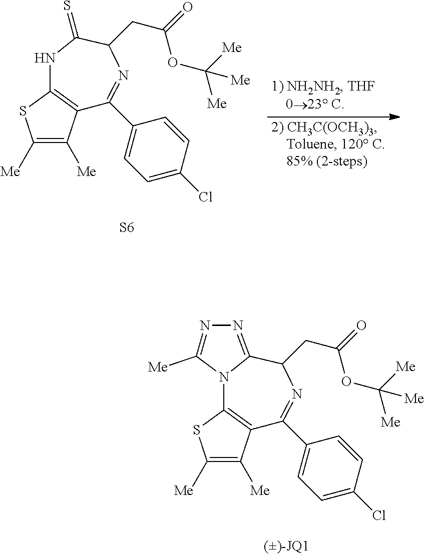

### (2-amino-4,5-dimethylthiophen-3-yl)(4-chlorophenyl)methanone (S2)

The compound JQ1 was prepared according to the scheme shown above.

Sulfur (220 mg, 6.9 mmol, 1.00 equiv) was added as a solid to a solution of 4-chlorobenzoyl acetonitrile S1 (1.24 g, 6.9 mmol, 1 equiv), 2-butanone (0.62 ml, 6.9 mmol, 1.00 equiv), and morpholine (0.60 ml, 6.9 mmol, 1.00 equiv) in ethanol (20 ml, 0.35 M) at 23° C.²¹. The mixture was then heated to 70° C. After 12 hours, the reaction mixture was cooled to 23° C. and poured into brine (100 ml). The aqueous layer was extracted with ethyl acetate (3×50 ml). The combined organic layers were washed with brine (50 ml), were dried over anhydrous sodium sulphate, were filtered, and were concentrated under reduced pressure. The residue was purified by flash column chromatography (Combiflash RF system, 40 gram silica gel, gradient 0 to 100% ethyl acetate-hexanes) to afford S2 (1.28 g, 70%) as a yellow solid.

### (S)-tert-Butyl-3-({[(9H-fluoren-9-yl)methoxy]carbonyl}amino)-4-{[3-(4-chlorobenzoyl)-4,5-dimethylthiophen-2-yl]amino}-4-oxobutanoate (S3)

(2-(6-Chloro-1H-benzotriazole-1-yl)-1,1,3,3-tetramethylaminium hexafluorophosphate (HCTU) (827 mg, 2.0 mmol, 2.00 equiv), and N,N-diisopropylethylamine (0.72 ml, 4.0 mmol, 4.00 equiv) were added sequentially to a solution of 9-fluorenylmethoxycarbonyl-aspartic acid β-tert-butyl ester [Fmoc-Asp(Ot-Bu)—OH] (864 mg, 2.1 mmol, 2.10 equiv) in N,N-dimethylformamide (1.5 ml, 1.0 M). The mixture was then stirred at 23° C. for 5 min. S2 (266 mg, 1.0 mmol, 1 equiv) was then added as a solid. The reaction mixture was stirred at 23° C. After 16 hours, ethyl acetate (20 ml) and brine (20 ml) were added. The two layers were separated, and the aqueous layer was extracted with ethyl acetate (2×20 ml). The combined organic layers were washed with brine (30 ml), were dried over with anhydrous sodium sulphate, were filtered, and were concentrated under reduced pressure. The residue was purified by flash column chromatography (Combiflash RF, 40 gram silica gel, gradient 0 to 100% ethyl acetate-hexanes) to afford S3 (625 mg, 90%) as brown oil.

### (S)-tert-butyl3-amino-4-((3-(4-chlorobenzoyl)-4,5-dimethylthiophen-2-yl)amino)-4-oxobutanoate (S4)

Compound S3 (560 mg, 0.85 mmol, 1 equiv) was dissolved into 20% piperidine in DMF solution (4.0 ml, 0.22 M) at 23° C. After 30 min, ethyl acetate (20 ml) and brine (20 ml) were added to the reaction mixture. The two layers were separated, and the aqueous layer was extracted with ethyl acetate (2×20 ml). The combined organic layers were washed with brine (3×25 ml), were dried over anhydrous sodium sulphate, were filtered, and were concentrated under reduced pressure. The residue was purified by flash column chromatography (Combiflash RF system, 24 gram silica gel, gradient 0 to 100% ethyl acetate-hexanes) to afford free amine S4 (370 mg, 90%) as yellow solid. The enantiomeric purity dropped to 75% (determined with Berger Supercritical Fluid Chromatography (SFC) using AS-H column).

### (S)-tert-Butyl 2-(5-(4-chlorophenyl)-6,7-dimethyl-2-oxo-2,3-dihydro-1H-thieno[2,3-e][1,4]diazepin-3-yl)acetate (S5)

Amino ketone (S4) (280 mg, 0.63 mmol) was dissolved in 10% acetic acid ethanol solution (21 ml, 0.03 M). The reaction mixture was heated to 85° C. After 30 minutes, all solvents were removed under reduced pressure. The residue was purified by flash column chromatography (Combiflash RF system, 12 gram silica gel, gradient 0 to 100% ethyl acetate-hexanes) to afford compound S5 (241 mg, 95%) as white solid. Enantiomeric purity of S5 was 67% (determined with Berger Supercritical Fluid Chromatography (SFC) using an AS-H column).

### tert-Butyl 2-(5-(4-chlorophenyl)-6,7-dimethyl-2-thioxo-2,3-dihydro-1H-thieno[2,3-e][1,4]diazepin-3-yl)acetate (S6)

Phosphorus pentasulfide (222 mg, 1.0 mmol, 2.00 equiv), sodium bicarbonate (168 mg, 2.0 mmol, 4.00 equiv) were added sequentially to a solution of S5 (210 mg, 0.5 mmol, 1 equiv) in diglyme (1.25 ml, 0.4M). The reaction mixture was heated to 90° C. After 16 h, brine (20 ml) and ethyl acetate (35 ml) were added. The two layers were separated, and the aqueous layer was extracted with ethyl acetate (3×30 ml). The combined organic layers were washed with brine (2×15 ml), were dried over anhydrous sodium sulphate, were filtered, and were concentrated under reduced pressure. The residue was purified by flash column chromatography (Combiflash RF system, 24 gram silica gel, gradient 0 to 100% ethyl acetate-hexanes) to afford S6 (141 mg, 65%) as brown solid with recovered S5 (73 mg, 34%).

### tert-Butyl 2-(4-(4-chlorophenyl)-2,3,9-trimethyl-6H-thieno[3,2-f][1,2,4]triazolo[4,3-a][1,4]diazepin-6-yl)acetate[(±)JQ1]

Hydrazine (0.015 ml, 0.45 mmol, 1.25 equiv) was added to a solution of S6 (158 mg, 0.36 mmol, 1 equiv) in THF (2.6 ml, 0.14 M) at 0° C. The reaction mixture was warmed to 23° C., and stirred at 23° C. for 1 h. All solvents were removed under reduced pressure. The resulting hydrazine was used directly without purification. The hydrazine was then dissolved in a 2:3 mixture of trimethyl orthoacetate and toluene (6 ml, 0.06 M). The reaction mixture was heated to 120° C. After 2 h, all the solvents were removed under reduced pressure. The residue was purified by flash column chromatography (Combiflash system, 4 g silica gel, gradient 0 to 100% ethyl acetate-hexanes) to afford JQ1 (140 mg, 85% in 2 steps) as white solid. The reaction conditions further epimerized the stereogenic center, resulting in the racemate, JQ1 (determined with Berger Supercritical Fluid Chromatography (SFC) with an AS-H column).

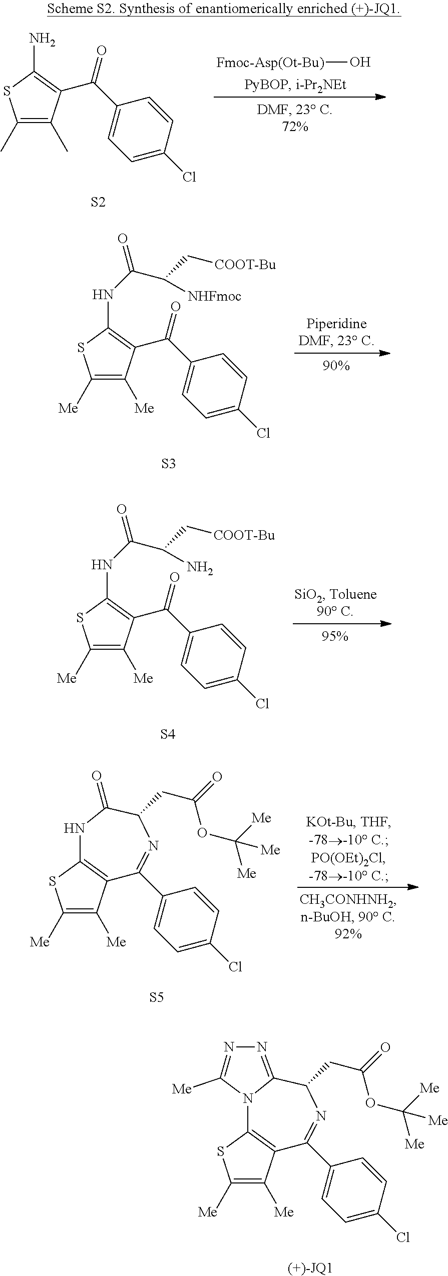

### (S)-tert-Butyl-3-({[(9H-fluoren-9-yl)methoxy]carbonyl}amino)-4-{[3-(4-chlorobenzoyl)-4,5-dimethylthiophen-2-yl]amino}-4-oxobutanoate (S3)

(Benzotriazol-1-yloxyl)tripyrrolidinophosphonium (PyBOP) (494 mg, 0.95 mmol, 0.95 equiv), N,N-diisopropylethylamine (0.50 ml, 2.8 mmol, 2.75 equiv) were added sequentially to a solution of 9-fluorenylmethoxycarbonyl-aspartic acid β-tert-butyl ester [Fmoc-Asp(Ot-Bu)—OH] (411 mg, 1.00 mmol, 1.0 equiv) in N,N-dimethylformamide (1.0 ml, 1.0 M). The mixture was then stirred at 23° C. for 5 min. S2 (266 mg, 1.0 mmol, 1 equiv) was then added as solid. The reaction mixture was stirred at 23° C. After 4 h, ethyl acetate (20 ml) and brine (20 ml) were added. The two layers were separated, and the aqueous layer was extracted with ethyl acetate (2×20 ml). The combined organic layers were washed with brine, were dried over with anhydrous sodium sulphate, were filtered, and were concentrated under reduced pressure. The residue was purified by flash column chromatography (Combiflash RF system, 40 gram silica gel, gradient 0 to 100% ethyl acetate-hexanes) to afford S3 (452 mg, 72%) as brown oil.

### (S)-tert-butyl3-amino-4-((3-(4-chlorobenzoyl)-4,5-dimethylthiophen-2-yl)amino)-4-oxobutanoate (S4)

Compound S3 (310 mg, 0.47 mmol, 1 equiv) was dissolved into 20% piperidine in DMF solution (2.2 ml, 0.22 M) at 23° C. After 30 min, ethyl acetate (20 ml) and brine (20 ml) were added to the reaction mixture. The two layers were separated, and the aqueous layer was extracted with ethyl acetate (2×20 ml). The combined organic layers were washed with brine (3×25 ml), were dried over anhydrous sodium sulphate, were filtered, and were concentrated under reduced pressure. The residue was purified by flash column chromatography (Combiflash RF system, 24 gram silica gel, gradient 0 to 100% ethyl acetate-hexane) to afford free amine S4 (184 mg, 90%) as yellow solid. The enantiomeric purity was 91% (checked with Berger Supercritical Fluid Chromatography (SFC) using an AS-H column).

### (S)-tert-Butyl 2-(5-(4-chlorophenyl)-6,7-dimethyl-2-oxo-2,3-dihydro-1H-thieno[2,3-e][1,4]diazepin-3-yl)acetate (S5)

Amino ketone (S4) (184 mg, 0.42 mmol) was dissolved in toluene (10 ml, 0.04 M). Silica gel (300 mg) was added, and the reaction mixture was heated to 90° C. After 3 h, the reaction mixture was cooled to 23° C. The silica gel was filtered, and washed with ethyl acetate. The combined filtrates were concentrated. The residue was purified by flash column chromatography (Combiflash RF system, 12 gram silica gel, gradient 0 to 100% ethyl acetate-hexanes) to afford compound S5 (168 mg, 95%) as white solid. Enantiomeric purity of S5 was 90% (determined with Berger Supercritical Fluid Chromatography (SFC) using an AS-H column).

### (S)-tert-Butyl 2-(4-(4-chlorophenyl)-2,3,9-trimethyl-6H-thieno[3,2-f][1,2,4]triazolo[4,3-a][1,4]diazepin-6-yl)acetate[(+)JQ1]

Potassium tert-butoxide (1.0 M solution in THF, 0.3 ml, 0.30 mmol, 1.10 equiv) was added to a solution of S5 (114 mg, 0.27 mmol, 1 equiv) in THF (1.8 ml, 0.15 M) at −78° C. The reaction mixture was warmed to −10° C., and stirred at 23° C. for 30 min. The reaction mixture was cooled to −78° C. Diethyl chlorophosphate (0.047 ml, 0.32 mmol, 1.20 equiv) was added to reaction mixture²². The resulting mixture was warmed to −10° C. over 45 min. Acetic hydrazide (30 mg, 0.40 mmol, 1.50 equiv) was added to reaction mixture. The reaction mixture was stirred at 23° C. After 1 h, 1-butanol (2.25 ml) was added to reaction mixture, which was heated to 90° C. After 1 h, all solvents were removed under reduce pressure. The residue was purified with flash column chromatography (Combiflash system, 4 g silica gel, gradient 0 to 100% ethyl acetate-hexanes) to afford (+)-JQ1 (114 mg, 92%) as white solid with 90% enantiomeric purity (determined with Berger Supercritical Fluid Chromatography (SFC) using AS-H column, 85% hexanes-methanol, 210 nm, t_{R} (R-enantiomer)=1.59 min, t_{R} (S-enantiomer)=3.67 min). The product was further purified by chiral preparative HPLC (Agilent High Pressure Liquid Chromatography using an OD-H column) to provide the S-enantiomer in greater than 99% ee.

¹H NMR (600 MHz, CDCl₃, 25° C.) δ 7.39 (d, J=8.4 Hz, 2H), 7.31 (d, J=8.4 Hz, 2H), 4.54 (t, J=6.6 MHz, 1H), 3.54-3.52 (m, 2H), 2.66 (s, 3H), 2.39 (s, 3H), 1.67 (s, 3H), 1.48 (s, 9H).

¹³C NMR (150 MHz, CDCl₃, 25° C.) δ 171.0, 163.8, 155.7, 150.0, 136.9, 131.1, 130.9, 130.6, 130.3, 128.9, 81.2, 54.1, 38.1, 28.4, 14.6, 13.5, 12.1.

HRMS (ESI) calc'd for C₂₁H₂₄ClN₂O₃S [M+H]⁺: 457.1460. found 457.1451 m/z.

TLC (EtOAc), Rf: 0.32 (UV)

[α]²²_{D}=+75 (c (0.5, CHCl₃)

(−)-JQ1 was synthesized in a similar manner, employing Fmoc-D-Asp(Ot-Bu)—OH as a starting material, and was further purified by chiral preparative HPLC (Agilent High Pressure Liquid Chromatography using an OD-H column) to afford the R-enantiomer in greater than 99% ee. [α]²²_{D}=−72 (c 0.5, CHCl₃)

### Synthesis of Additional Compounds

Additional compounds of the invention were prepared as illustrated in Scheme S3.

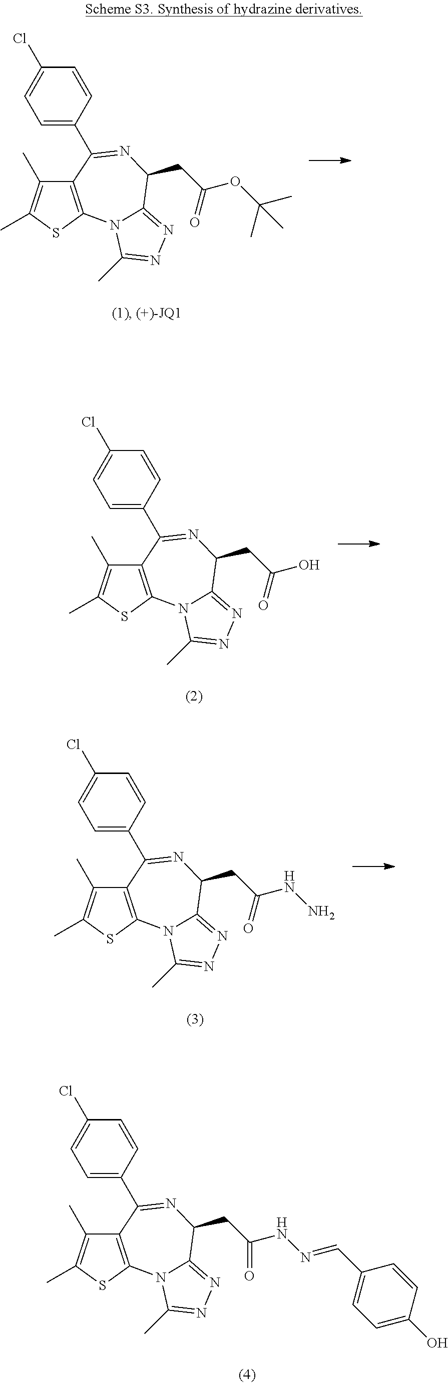

As shown in Scheme S3, the t-butyl ester of (+)-JQ1 (1) was cleaved to yield the free acid (2), which was coupled with hydrazine to yield the hydrazide (3). Reaction with 4-hydroxybenzaldehyde yielded the hydrazone (4).

Both hydrazide (3) and hydrazone (4) showed activity in at least one biological assay.

A library of compounds was prepared by reaction of the hydrazide (3) with a variety of carbonyl-containing compounds (see Table A, above).

Additional compounds were prepared for use, e.g., as probes for assay development. An exemplary synthesis is shown in Scheme S4, below.

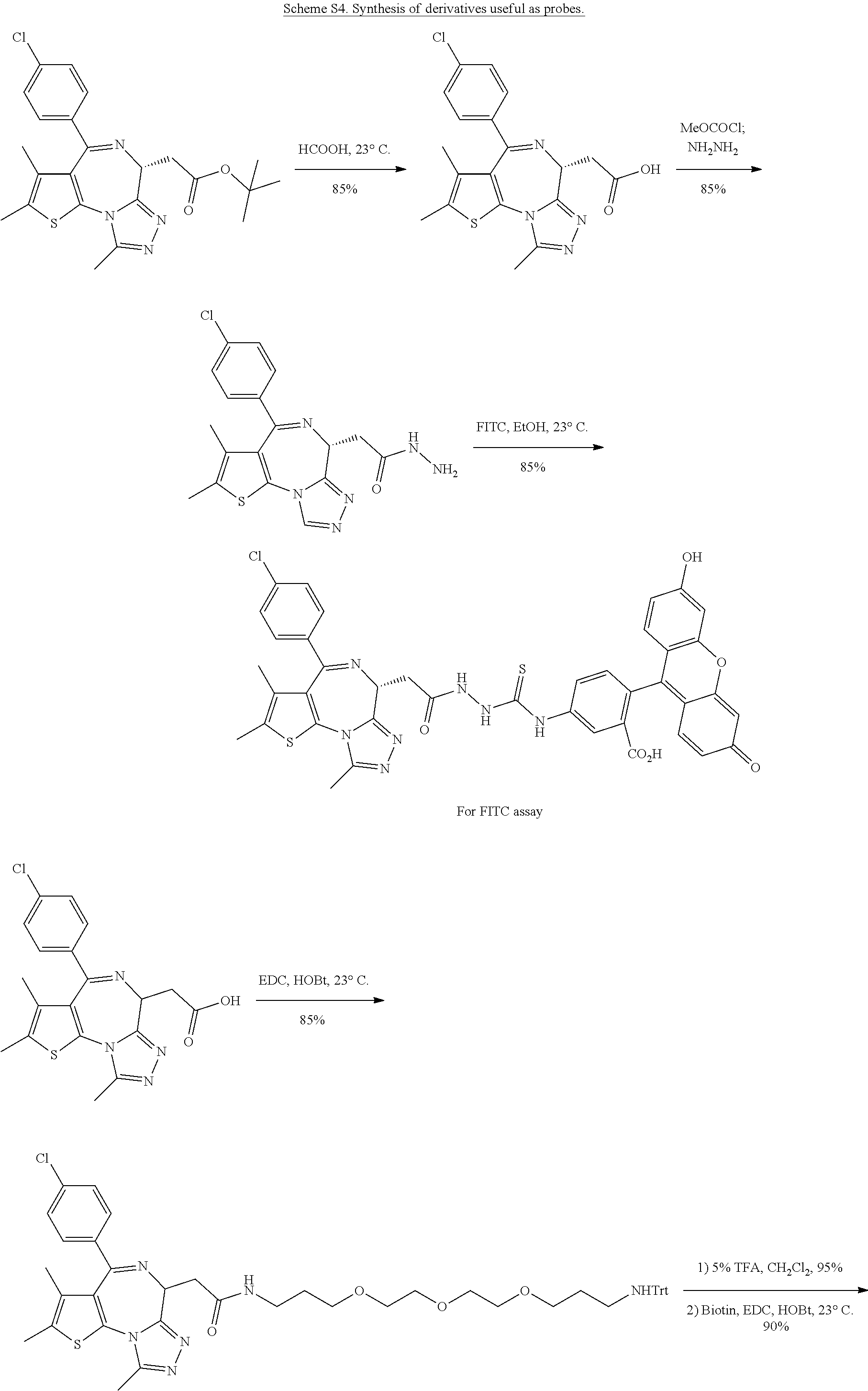

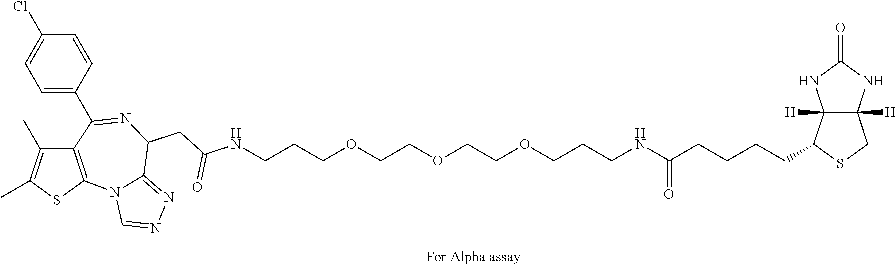 [SMILES: Cc1sc2c(c1C)C(c1ccc(Cl)cc1)=NC(CC(=O)NCCCOCCOCCOCCCNC(=O)CCCC[C@H]1SC[C@H]3NC(=O)N[C@H]31)c1nncn1-2]

Additional compounds were prepared as shown in the table below:

| | | MS |
|---|---|---|
| | | [M + H]⁺ |
| Compound | | m/z |
| Name | Structure | (Observed) |
| (S)-JQ1 | 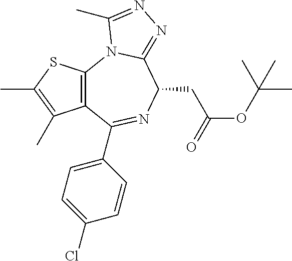 [SMILES: Cc1sc2c(c1C)C(c1ccc(Cl)cc1)=N[C@@H](CC(=O)OC(C)(C)C)c1nnc(C)n1-2] | 457.1 |
| (R)-JQ1 | 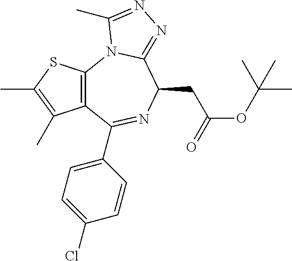 [SMILES: Cc1sc2c(c1C)C(c1ccc(Cl)cc1)=N[C@H](CC(=O)OC(C)(C)C)c1nnc(C)n1-2] | 457.1 |
| JQ3 | 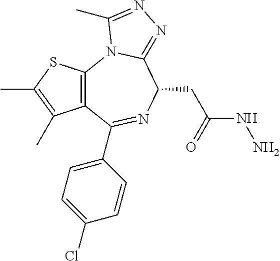 [SMILES: Cc1sc2c(c1C)C(c1ccc(Cl)cc1)=N[C@@H](CC(=O)NN)c1nnc(C)n1-2] | 415.1 |
| JQ4 | 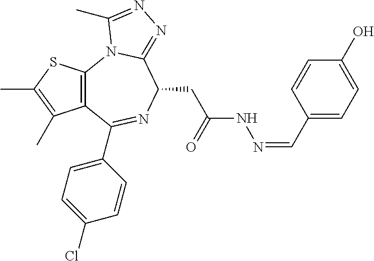 [SMILES: Cc1sc2c(c1C)C(c1ccc(Cl)cc1)=N[C@@H](CC(=O)N/N=C\c1ccc(O)cc1)c1nnc(C)n1-2] | 519.1 |
| JQ6 | 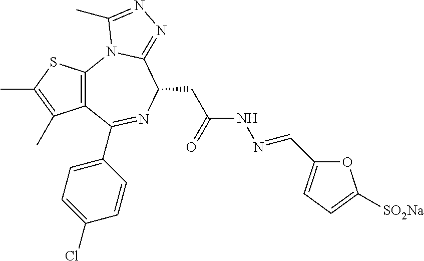 | 493.1 |
| JQ7 | 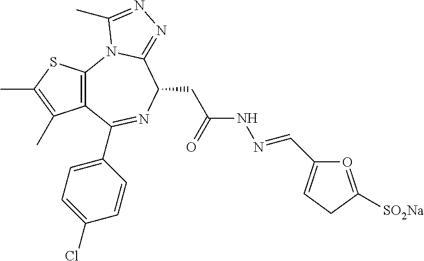 | 579.0 |
| JQ8 | 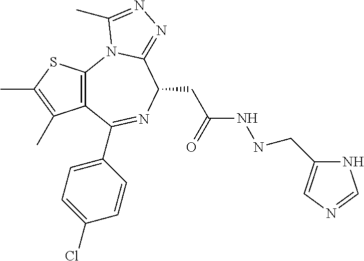 [SMILES: Cc1sc2c(c1C)C(c1ccc(Cl)cc1)=N[C@@H](CC(=O)NNCc1cnc[nH]1)c1nnc(C)n1-2] | 494.1 |
| JQ10 | 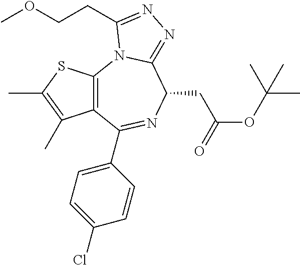 [SMILES: COCCc1nnc2n1-c1sc(C)c(C)c1C(c1ccc(Cl)cc1)=N[C@H]2CC(=O)OC(C)(C)C] | 501.1 |
| JQ11 | 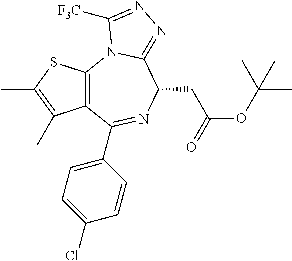 [SMILES: Cc1sc2c(c1C)C(c1ccc(Cl)cc1)=N[C@@H](CC(=O)OC(C)(C)C)c1nnc(C(F)(F)F)n1-2] | 511.1 |
| JQ1-FITC | 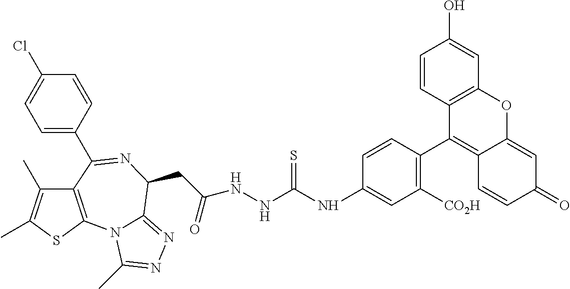 [SMILES: Cc1sc2c(c1C)C(c1ccc(Cl)cc1)=N[C@@H](CC(=O)NNC(=S)Nc1ccc(-c3c4ccc(=O)cc-4oc4cc(O)ccc34)c(C(=O)O)c1)c1nnc(C)n1-2] | 804.1 |
| JQ1-Biotin | 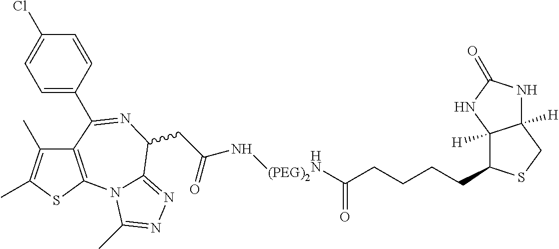 | 829.3 |
| JQ13 | 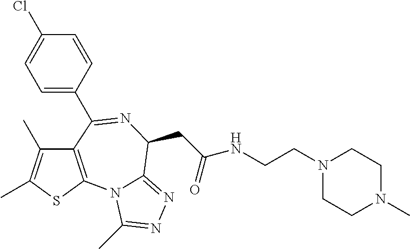 | 526.2 |
| KS1 | 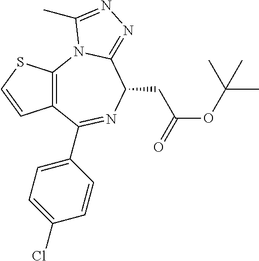 [SMILES: Cc1nnc2n1-c1sccc1C(c1ccc(Cl)cc1)=N[C@H]2CC(=O)OC(C)(C)C] | 429.1 |
| JQ18 | 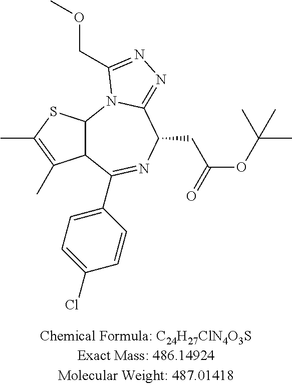 [SMILES: COCc1nnc2n1C1SC(C)=C(C)C1C(c1ccc(Cl)cc1)=N[C@H]2CC(=O)OC(C)(C)C] | 487.1 |
| JQ19 | 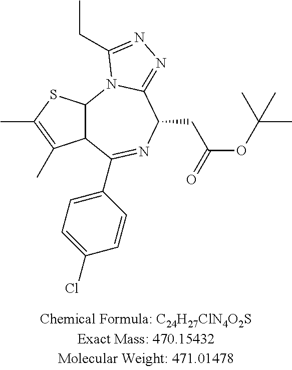 [SMILES: CCc1nnc2n1C1SC(C)=C(C)C1C(c1ccc(Cl)cc1)=N[C@H]2CC(=O)OC(C)(C)C] | 471.1 |
| JQ20 | 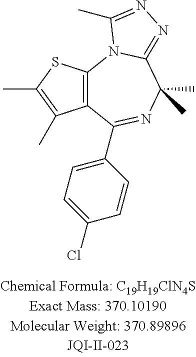 | 370.1 |
| JQ21 | 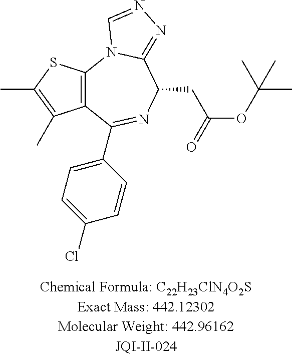 [SMILES: Cc1sc2c(c1C)C(c1ccc(Cl)cc1)=N[C@@H](CC(=O)OC(C)(C)C)c1nncn1-2] | 443.1 |
| JQ24A | 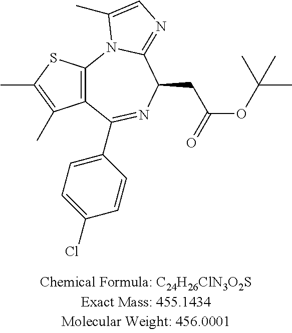 [SMILES: Cc1sc2c(c1C)C(c1ccc(Cl)cc1)=N[C@H](CC(=O)OC(C)(C)C)c1ncc(C)n1-2] | 456.1 |
| JQ24B | 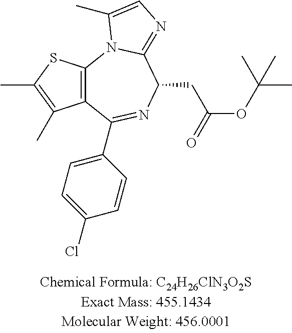 [SMILES: Cc1sc2c(c1C)C(c1ccc(Cl)cc1)=N[C@@H](CC(=O)OC(C)(C)C)c1ncc(C)n1-2] | 456.1 |
| JQ25 | 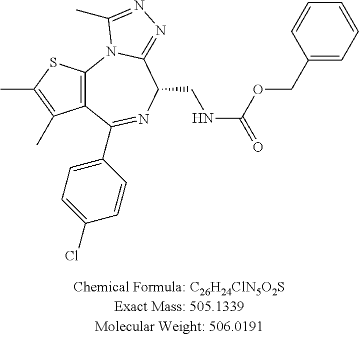 [SMILES: Cc1sc2c(c1C)C(c1ccc(Cl)cc1)=N[C@@H](CNC(=O)OCc1ccccc1)c1nnc(C)n1-2] | 506.1 |
| JQB | 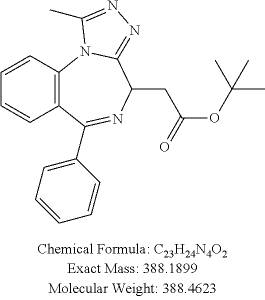 [SMILES: Cc1nnc2n1-c1ccccc1C(c1ccccc1)=NC2CC(=O)OC(C)(C)C] | 389.2 |
| JQ30 | 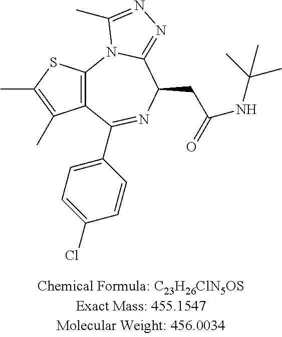 [SMILES: Cc1sc2c(c1C)C(c1ccc(Cl)cc1)=N[C@H](CC(=O)NC(C)(C)C)c1nnc(C)n1-2] | 456.2 |
| JQ31 | 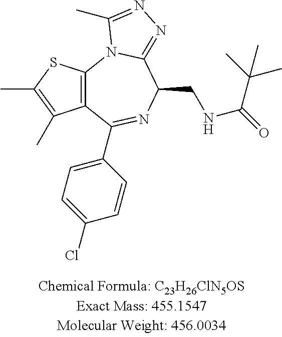 [SMILES: Cc1sc2c(c1C)C(c1ccc(Cl)cc1)=N[C@H](CNC(=O)C(C)(C)C)c1nnc(C)n1-2] | 456.2 |
| JQ32 | 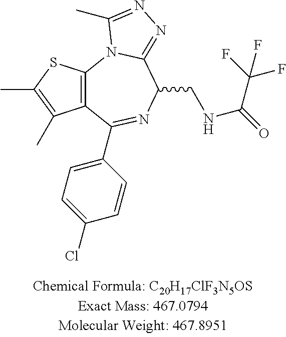 [SMILES: Cc1sc2c(c1C)C(c1ccc(Cl)cc1)=NC(CNC(=O)C(F)(F)F)c1nnc(C)n1-2] | 468.1 |
| JQ33 | 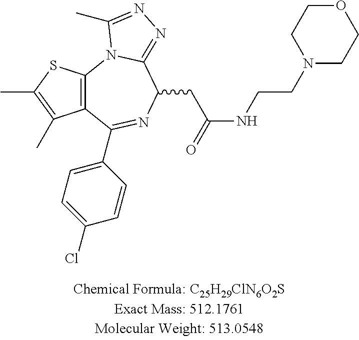 [SMILES: Cc1sc2c(c1C)C(c1ccc(Cl)cc1)=NC(CC(=O)NCCN1CCOCC1)c1nnc(C)n1-2] | 512.2 |
| JQ34 | 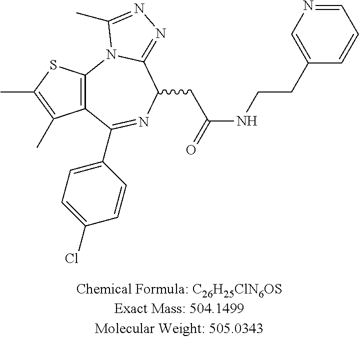 [SMILES: Cc1sc2c(c1C)C(c1ccc(Cl)cc1)=NC(CC(=O)NCCc1cccnc1)c1nnc(C)n1-2] | 505.1 |
| JQ35 | 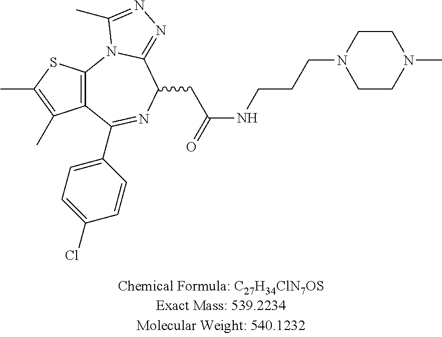 | 540.2 |
| JQ36 | 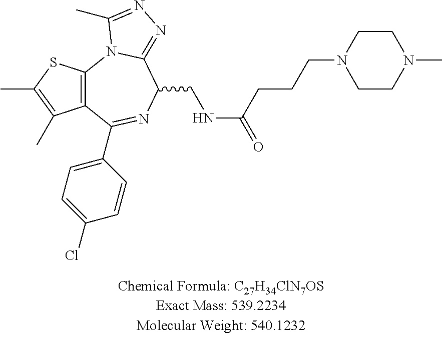 | 540.2 |
| JQ37 | 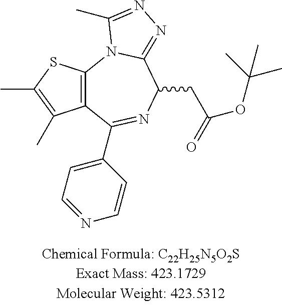 [SMILES: Cc1sc2c(c1C)C(c1ccncc1)=NC(CC(=O)OC(C)(C)C)c1nnc(C)n1-2] | 424.2 |
| JQ38 | 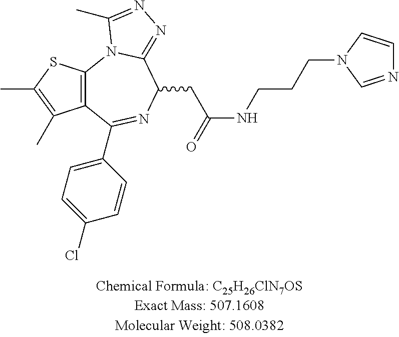 [SMILES: Cc1sc2c(c1C)C(c1ccc(Cl)cc1)=NC(CC(=O)NCCCn1ccnc1)c1nnc(C)n1-2] | 508.2 |
| JQ39 | 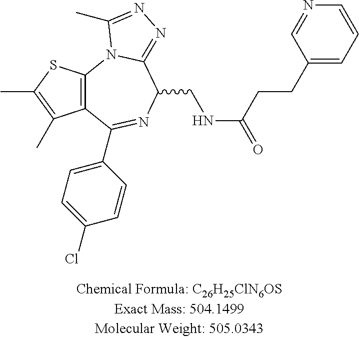 [SMILES: Cc1sc2c(c1C)C(c1ccc(Cl)cc1)=NC(CNC(=O)CCc1cccnc1)c1nnc(C)n1-2] | 505.1 |
| JQ40 | 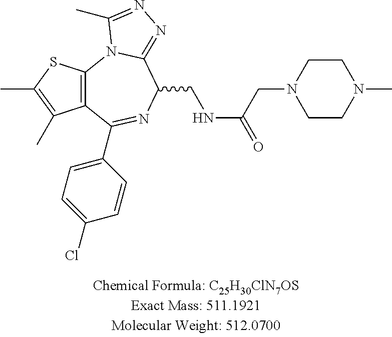 | 512.2 |
| JQ41 | 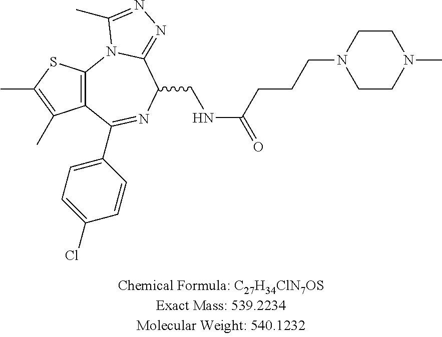 | 540.2 |
| JQ42 | 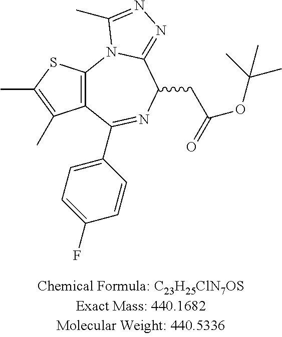 [SMILES: Cc1sc2c(c1C)C(c1ccc(F)cc1)=NC(CC(=O)OC(C)(C)C)c1nnc(C)n1-2] | 441.2 |
| JQ43 | 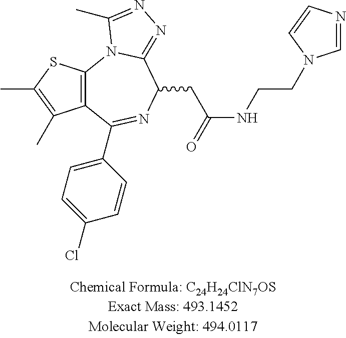 [SMILES: Cc1sc2c(c1C)C(c1ccc(Cl)cc1)=NC(CC(=O)NCCn1ccnc1)c1nnc(C)n1-2] | 494.1 |
| JQ44 | 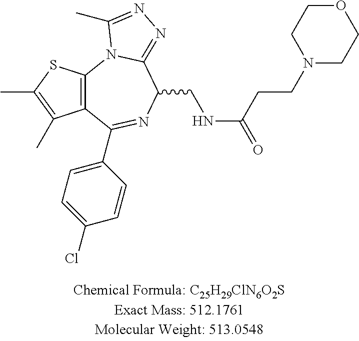 [SMILES: Cc1sc2c(c1C)C(c1ccc(Cl)cc1)=NC(CNC(=O)CCN1CCOCC1)c1nnc(C)n1-2] | 513.2 |
| JQ45 | 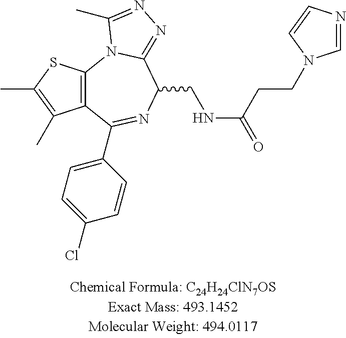 [SMILES: Cc1sc2c(c1C)C(c1ccc(Cl)cc1)=NC(CNC(=O)CCn1ccnc1)c1nnc(C)n1-2] | 494.1 |
| JQ46 | 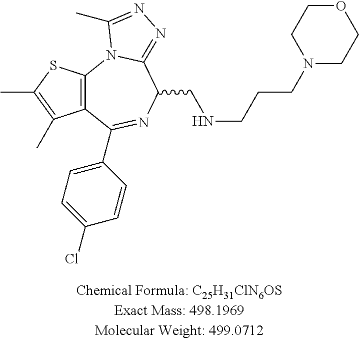 [SMILES: Cc1sc2c(c1C)C(c1ccc(Cl)cc1)=NC(CNCCCN1CCOCC1)c1nnc(C)n1-2] | 499.2 |
| JQ47 | 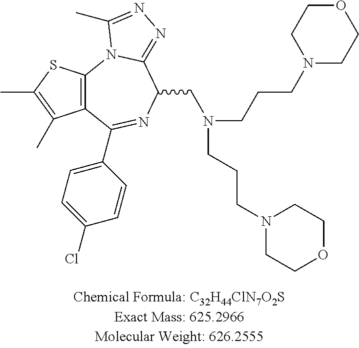 [SMILES: Cc1sc2c(c1C)C(c1ccc(Cl)cc1)=NC(CN(CCCN1CCOCC1)CCCN1CCOCC1)c1nnc(C)n1-2] | 626.3 |
| JQ48 | 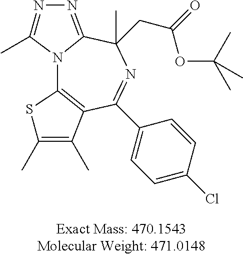 [SMILES: Cc1sc2c(c1C)C(c1ccc(Cl)cc1)=NC(C)(CC(=O)OC(C)(C)C)c1nnc(C)n1-2] | 471.2 |
| JQ49 | 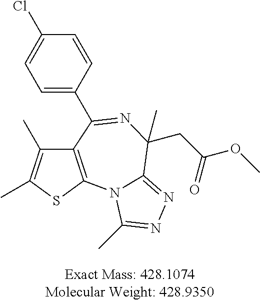 | 429.1 |
| JQ50 | 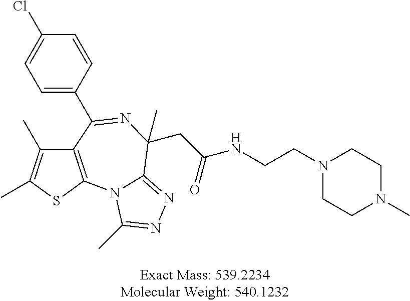 | 540.2 |
| JQ51 | 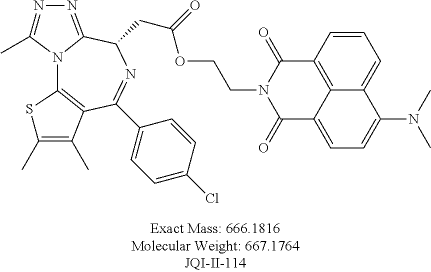 | 667.2 |
| JQ52 | 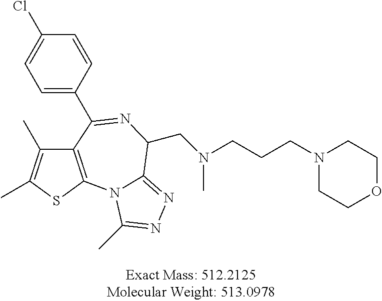 [SMILES: Cc1sc2c(c1C)C(c1ccc(Cl)cc1)=NC(CN(C)CCCN1CCOCC1)c1nnc(C)n1-2] | 513.2 |
| JQ53 | 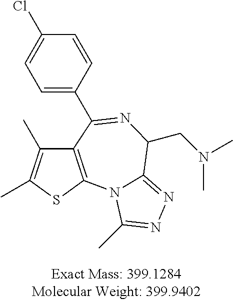 | 400.1 |

Spectral data for each compound were consistent with the assigned structure.

### II. Biological Activity and Methods of Treatment

### Example 1

### Inhibition of BET Protein Family Members Blocks Adipogenesis

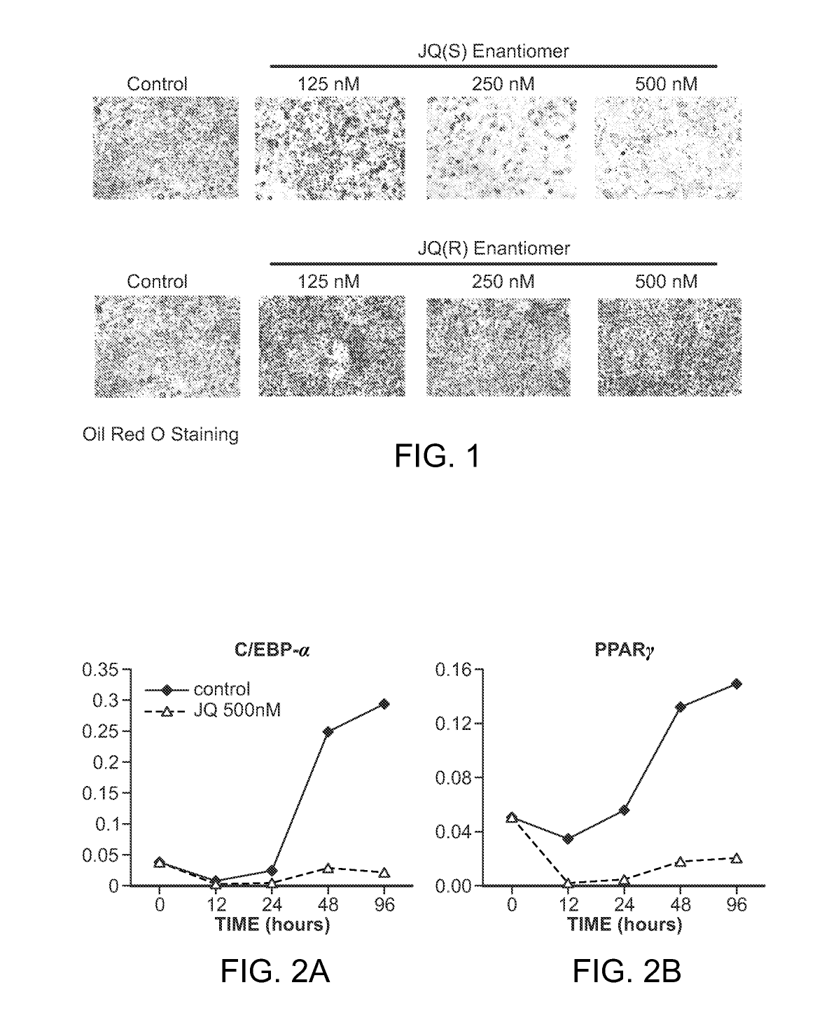

3T3L1 cells are a well characterized cell type that can be differentiated into fat cells that contain large lipid droplets. In this experiment, 3T3L1 cells were differentiated in the presence of increasing concentrations of a chemical inhibitor of BET family proteins JQ (FIG. 1, top panels) or an inactive version of the inhibitor (FIG. 1, bottom panels). The cells were differentiated for eight days. On the final day, the cells were stained with Oil Red O, which stains for lipid accumulation in the cells. As shown in FIG. 1, treatment with the JQ (S) enantiomer inhibited adipogenesis in a dose dependent manner, whereas the inactive JQ (R) enantiomer had no effect on the generation of lipid. The inhibition of BET proteins significantly reduced lipid accumulation as measured by loss of red staining in the cells, and this effect was dose dependent.

### Example 2

### Inhibition of BET Protein Family Members Blocks Expression of C/EBPα and PPARγ in 3T3L1 Cells During Adipocyte Differentiation

3T3L1 cells were differentiated in the presence or absence of a chemical inhibitor of the BET protein family (500 nM of JQ). Gene expression levels of two key proteins that function in fat cell differentiation, C/EBPα and PPARγ, were measured over the first four days of differentiation. As shown in FIGS. 2A and 2B, inhibition of BET proteins significantly blocked the induction of C/EBPα and PPARγ expression.

### Example 3

### Inhibition of BET Protein Family Members Blocks Weight Gain in a Mouse Model of Obesity

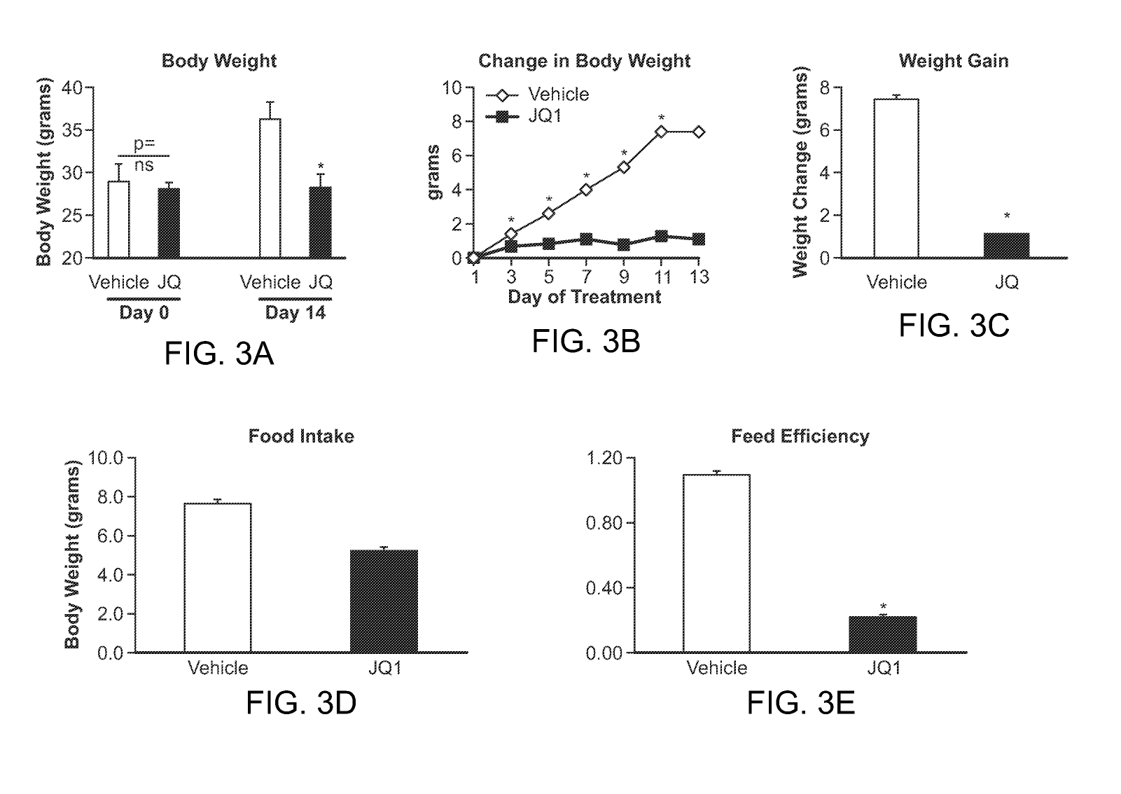

Ob/ob mice are a well established mouse obesity model that gains weight rapidly on a normal mouse chow diet. Five week old Ob/ob mice were treated with vehicle (control) or a BET protein family inhibitor (JQ) for 14 days. As shown in FIGS. 3A-3C, treatment with 50 mg/kg JQ blocked weight gain in ob/ob mice. JQ treatment also mildly inhibited food intake and feed efficiency as shown in FIGS. 3D and 3E respectively. The reduction in feed efficiency with the BET protein family inhibitor indicates that food intake cannot explain the difference in body weight. Without wishing to be bound by theory, it is likely that the disparity is due to differences in the way that ob/ob mice treated with JQ are metabolizing food relative to untreated ob/ob mice.

### Example 4

### Inhibition of BET Protein Family Members Reduces Liver and Adipose Tissue Weight in Ob/Ob Mice

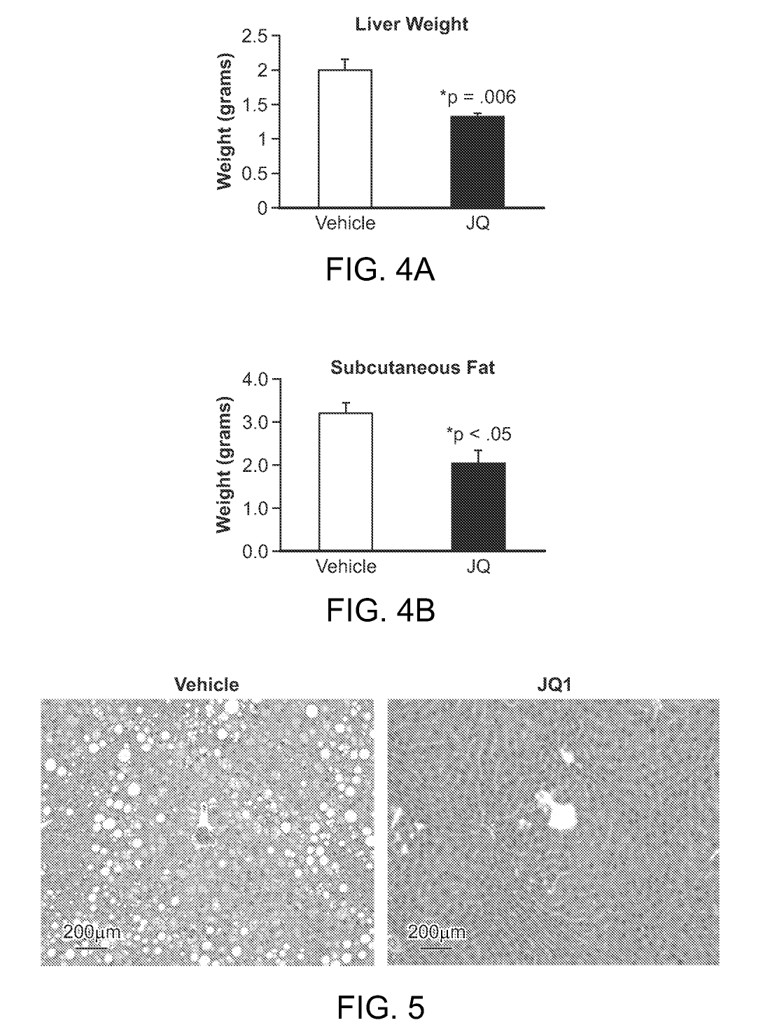

Five week old Ob/ob mice were treated with either vehicle (control) or a chemical inhibitor of the BET protein family (JQ) for approximately two weeks. Following treatment the mice were euthanized and organs were harvested and weighed. The weight of the livers and subcutaneous fat were determined. As shown in FIGS. 4A and 4B, the group of mice treated with the BET protein family inhibitor demonstrated statistically significant reductions in liver (FIG. 4A) and subcutaneous fat weight (FIG. 4B). In addition, following organ harvest, liver was sectioned and stained with H&E. In the vehicle treated mice, significant lipid droplets were present in the liver as demonstrated by the abundant, large white droplets within the cells (FIG. 5). This finding is consistent with hepatic steatosis or fatty liver. In contrast, the mice treated with the BET protein inhibitor revealed a complete block in hepatic steatosis following the two week treatment. The histology of livers of JQ treated mice were morphologically normal and revealed a total absence of lipid accumulation in the liver. This indicates that treatment with JQ was able to reverse liver steatosis.

### Example 5

### Inhibition of BET Protein Family Members Reduced the Expression of Genes That Control Fat Accumulation in Liver

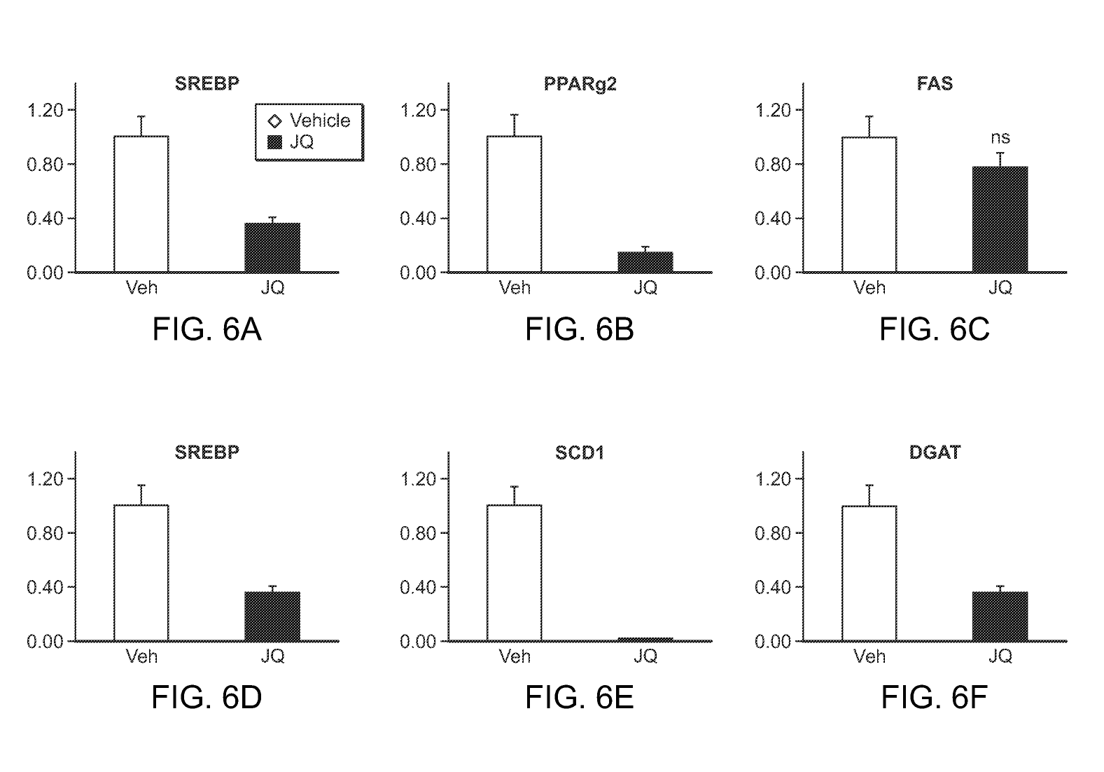

Five week old Ob/ob mice were treated for two weeks with a BET protein family inhibitor. Following treatment, the organs were harvested and liver RNA was isolated to measure gene expression profiles. The expression levels of a panel of genes that function in the control of fat accumulation in liver were measured. Consistent with the histology demonstrating decreased fat accumulation in the liver, inhibition of the BET protein family significantly reduced the expression of sterol regulatory binding protein (“SREBP”) (FIG. 6A), peroxisome proliferator activated receptor 2 (“PPARg2”) (FIG. 6B), fatty acid synthase (“FAS”) (FIG. 6C), acetyl CoA carboxylase beta (“ACC beta”) (FIG. 6D), stearoyl CoA desaturase 1 (“SCD1”) (FIG. 6E), and diacylglycerol acyl transferase 1 (“DGAT”) (FIG. 6F).

### Example 6

### Bromodomain Inhibition Reduced Visceral Fat Mass in Mice Fed a Normal Chow Diet

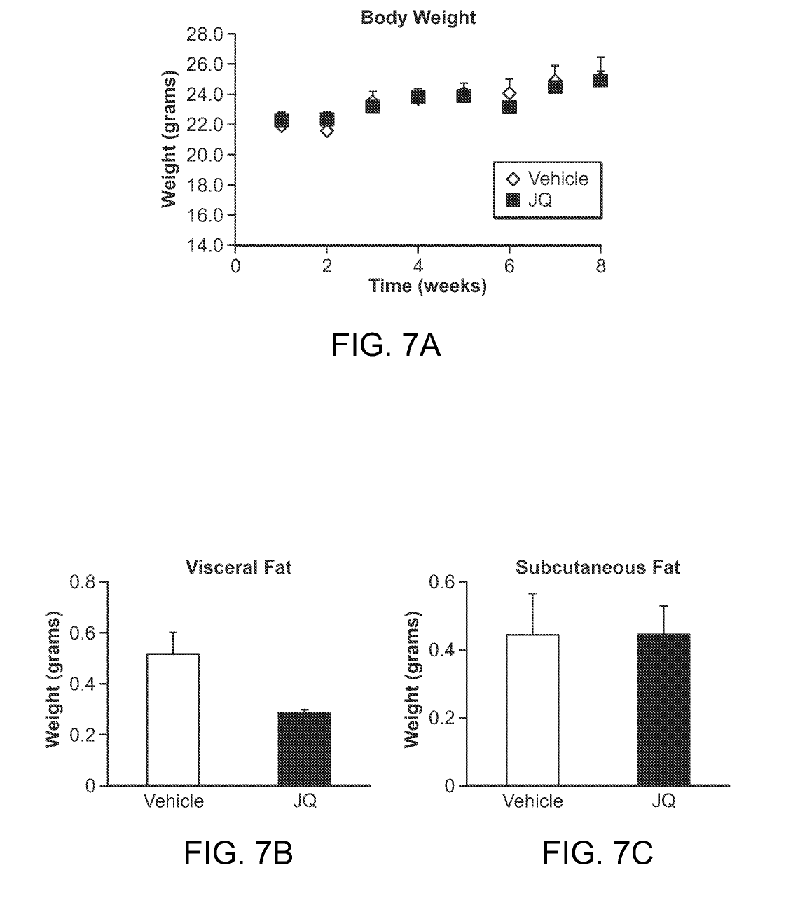

8 week old C57B/6 male mice were fed a standard chow diet for 8 weeks. These mice were also started on treatment with the bromodomain inhibitor JQ1 at 50 mg/kg or vehicle control administered by once daily intraperitoneal injection. As show in FIGS. 7A-7C, after 8 weeks on treatment the JQ1-treated mice demonstrated a significant reduction in epididymal adipose tissue mass (FIG. 7B), while overall body weight (FIG. 7A) and subcutaneous adipose tissue (FIG. 7C) were similar to vehicle treated animals.

### Example 7

### Bromodomain Inhibition Blocked Weight Gain in Response to High Fat Diet

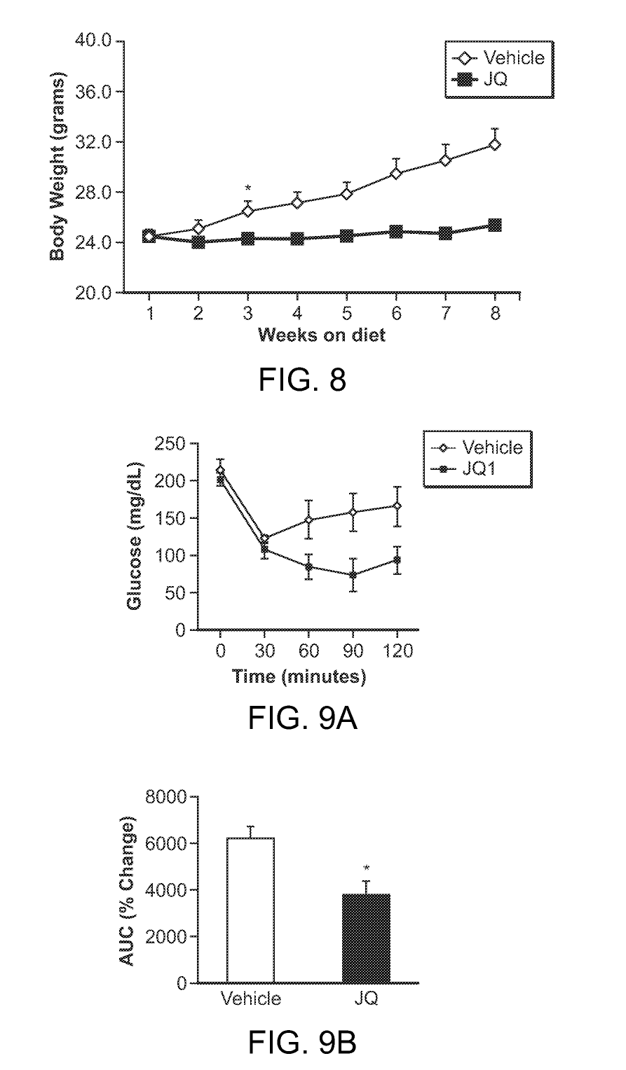

8 week old C57B/6 male mice were started on a high fat diet containing 60% kcal fat. These mice were also started on treatment with the bromodomain inhibitor JQ1 at 50 mg/kg or vehicle control administered by once daily intraperitoneal injection. Body weight was measured every week. As show in FIG. 8, the vehicle treated mice gained nearly 10 grams during this 8 week dietary challenge; however, treatment with JQ1 blocked this increase in body weight and the JQ1 treated mice remained lean. The weight curves separate in a statistically significant way after 3 weeks on treatment (*p<0.05).

### Example 8

### Bromodomain Inhibition Protected Against Insulin Resistance after Exposure to a High Fat Diet

8 week old C57B/6 male mice were started on a high fat diet containing 60% kcal fat. These mice were also started on treatment with the bromodomain inhibitor JQ1 at 50 mg/kg or vehicle control administered by once daily intraperitoneal injection. Body weight was measured every week. The mice were then examined for the degree of insulin resistance by insulin tolerance testing. After 7 weeks on high fat diet and simultaneous JQ1 or vehicle treatment, mice were fasted for 4 hours and then administered a single bolus of insulin (0.5 U/kg) by intraperitoneal injection. Following insulin injection blood glucose was measured at the indicated time points. As shown in FIG. 9A, vehicle treated mice demonstrated insulin resistance as shown by the rapid return of blood glucose back to starting levels. In contrast, the JQ treated mice showed a sustained decrease in blood glucose up to 2 hours after insulin injection, demonstrating a heightened response to insulin and clearance of blood glucose (FIG. 9A). As shown in FIG. 9B, the area under the curve for change in blood glucose revealed a statistically significant decrease in glucose during this 2 hour time course (p<0.05).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The subject matter described herein may be related to subject matter of U.S. provisional applications 61/334,991, 61/370,745, and 61/375,663, each of which is incorporated herein by this reference.

1. A method of inhibiting adipogenesis, the method comprising contacting an adipocyte or pre-adipocyte with an effective amount of an agent that inhibits a bromodomain and extra-terminal (BET) protein.

2. The method of claim 1, wherein the method inhibits adipocyte differentiation, proliferation, or hypertrophy.

3. A method of inhibiting adipocyte biological function, the method comprising contacting an adipocyte with an effective amount of an agent that inhibits a bromodomain and extra-terminal (BET) protein.

4. The method of claim 3, wherein the method reduces fatty acid synthesis, lipogenesis, lipid droplet accumulation.

5. A method for treating or preventing metabolic syndrome in a subject, the method comprising administering to the subject an effective amount of an agent that inhibits a bromodomain and extra-terminal (BET) protein, thereby treating or preventing metabolic syndrome in the subject.

6. The method of claim 5, wherein the method reduces abdominal obesity, atherogenic dyslipidemia, elevated blood pressure, insulin resistance, or type II diabetes.

7. A method for treating or preventing obesity or weight gain in a subject, the method comprising administering to the subject an effective amount of an agent that inhibits a bromodomain and extra-terminal (BET) protein, thereby treating or preventing obesity or weight gain in the subject.

8. A method of inhibiting hepatic steatosis in a subject, the method comprising administering to the subject an effective amount of an agent that inhibits a bromodomain and extra-terminal (BET) protein, thereby inhibiting hepatic steatosis.

9. A method of reducing subcutaneous fat or visceral fat in a subject, the method comprising administering to the subject an effective amount of an agent that inhibits a bromodomain and extra-terminal (BET) protein, thereby reducing subcutaneous fat or visceral fat in the subject.

10. A method of inhibiting food intake or increasing metabolism in a subject, the method comprising administering to the subject an effective amount of an agent that inhibits a bromodomain and extra-terminal (BET) protein, thereby inhibiting food intake or increasing metabolism in the subject.

11. A method of protecting against insulin resistance in a subject, the method comprising administering to the subject an effective amount of an agent that inhibits a bromodomain and extra-terminal (BET) protein, thereby protecting against insulin resistance in the subject.

12. The method of claim 1, wherein the agent is a compound of any of Formulas I-XXII, or any compound disclosed herein, or a derivative thereof.

13. The method of claim 12, wherein the compound is JQ1.

14. The method of claim 1, wherein the agent is an inhibitory nucleic acid molecule.

15. The method of claim 14, wherein the inhibitory nucleic acid molecule is an siRNA, shRNA or antisense nucleic acid molecule that reduces the expression of Brd2, Brd3, or Brd4.

16. The method of claim 1, wherein the bromodomain and extra-terminal (BET) protein is Brd2, Brd3, or Brd4.

17. The method of claim 1, wherein the method reduces the level of a C/EBPα and/or PPARγ polypeptide or polynucleotide.

18. The method of claim 1, wherein the method reduces the level of a sterol regulatory binding protein (SREBP), peroxisome proliferator activated receptor 2 (PPARg2), fatty acid synthase (FAS), acetyl CoA carboxylase beta, stearoyl CoA desaturase 1 (SCD1), and diacyglycerol acyl transferase 1 (DGAT).

19. The method of claim 1, wherein the agent is administered locally or systemically.

20. A kit for the treatment of a body weight disorder, the kit comprising an effective amount of an inhibitor of bromodomain and extra-terminal (BET) protein and direction for use of the kit in the method of claim 1.

21. The kit of claim 20, wherein the inhibitor of bromodomain and extra-terminal (BET) protein is JQ1.
